(19) United States
(12) Patent Application Publication
(10) Pub. No.: US 2025/0170268 A1
(43) Pub. Date: May 29, 2025

(54) METHODS AND TOOLS FOR CONJUGATION TO ANTIBODIES

(71) Applicant: Merck Patent GmbH, Darmstadt (DE)

(72) Inventors: Stephan DICKGIESSER, Darmstadt (DE); Nicolas RASCHE, Darmstadt (DE); Marcel RIEKER, Weiterstadt (DE)

(21) Appl. No.: 18/844,321

(22) PCT Filed: Mar. 9, 2023
(86) PCT No.: PCT/EP2023/056080

§ 371 Date: Sep. 5, 2024

(30) Foreign Application Priority Data
Mar. 9, 2022 (EP) EP22161191.6

(51) Int. Cl. A61K 47/68; A61K 47/54; C07K 16/28; C07K 16/30; C12N 9/10

(52) U.S. Cl. CPC A61K 47/6889; A61K 47/549; A61K 47/68031; A61K 47/68037; A61K 47/6849; A61K 47/6853; C07K 16/2887; C07K 16/3007; C12N 9/1044; C12Y 203/02013; C07K 2319/20

(57) ABSTRACT

The present invention relates to antibodies comprising tags that can be covalently conjugated to the primary amino group of a linker in the presence of a microbial transglutaminase. Also provided by the invention are methods for producing an antibody-linker-conjugate by mixing an antibody with a linker and a microbial transglutaminase. Also provided are antibody-linker conjugates and antibody-drug-conjugates producible according to the inventive method and a kit of parts comprising a buffer and a microbial transglutaminase.

### TECHNICAL FIELD

The present invention relates to antibodies comprising tags that can be covalently conjugated to the primary amino group of a linker in the presence of a microbial transglutaminase. Also provided by the invention are methods for producing an antibody-linker-conjugate by mixing an antibody with a linker and a microbial transglutaminase. Also provided are antibody-linker conjugates and antibody-drug-conjugates producible according to the inventive method and a kit of parts comprising a buffer and a microbial transglutaminase.

### BACKGROUND

Antibody-drug conjugates or ADCs are a class of biopharmaceutical drugs designed as a targeted therapy e.g. for treating cancer (Hamilton G S (September 2015). “Antibody-drug conjugates for cancer therapy: The technological and regulatory challenges of developing drug-biologic hybrids”. Biologicals. 43 (5): 318-32). Unlike chemotherapy, ADCs are intended to specifically treat the target cells while minimizing drug-exposure for healthy cells. A stable link between the antibody and drug (e.g. cytotoxic (anti-cancer) agent) is an important aspect of an ADC. A stable ADC linker ensures that less of the cytotoxic payload falls off before reaching a target cell, e.g. tumor cell, improving safety, and limiting dosages. The drug that is linked via the linker to the antibody includes therapeutic agents such as therapeutic agents for the treatment of cancer, e.g. for different solid tumor indications including e.g. CRC, pancreatic cancer, gastric cancer, NSCLC, esophageal cancer and prostate cancer.

The generation of antibody-drug conjugates (ADCs) involves a bioconjugation step that is required to attach a chemically synthesized small molecular drug, an API (active pharmaceutical ingredient), to a biotechnologically produced targeting moiety like an antibody. Attaching APIs to monoclonal antibodies (mAbs) is preferably done in a strictly controlled manner with regard to conjugation site and number of drugs attached (specific drug-to-antibody ratio, DAR). One way of obtaining such site-specific conjugates is via utilization of microbial transglutaminase (mTG, Tgase). However, conjugation of APIs using mTG typically requires prior modification of the mAb, either e.g. via genetic engineering or removal of the natural glycosylation. These modifications prevent using native mAbs directly and can lead to negative effects regarding certain characteristics of the final ADC product.

To overcome such negative effects and allow more flexibility with regards to ADC design, new technologies are needed. In view of the above, there remains a need for new and improved antibody-linker conjugation methods that provide higher drug-to-antibody ratios (DARs).

### SUMMARY OF THE INVENTION

A transglutaminase from S. ladakanum has been described in U.S. Pat. No. 6,660,510 B2. Methods for conjugation of native mAb using transglutaminases are mentioned in WO 2019/057772 and WO 2020/188061 A1. However, these publications do not contain sufficient information on ideal reaction buffer conditions and lack further means to maximize the conjunction efficiency, including the preferred conjugation tags that can be used in the antibodies that are provided by the present invention.

The present invention addresses this need and provides methods to achieve site-specific conjugation of two or more linkers (e.g. drug-linkers) to fully glycosylated antibodies. One targeted conjugation site is glutamine Q295 (Eu numbering) in the heavy chain of antibodies, which is very close to the typical N-glycosylation site N297. Conjugation to this position 295 is facilitated by using a certain transglutaminase, certain reaction conditions, specific mutations or insertions (such as glutamine-containing peptide tags) in the antibody sequence or a combination of the former. In some cases, conjugation is not only targeted to Q295 but simultaneously to additional positions to produce ADCs with e.g. 4 or 6 drug-linkers attached. These additional positions are either positioned in peptide tags fused to or integrated into the antibodies. Some of the ADCs with a drug-to-antibody ratio of 2 or higher show exquisite pharmacological in vivo characteristics (PK).

While the use of peptide tags fused to mAbs as MTG recognition motifs has been described, it was an unexpected finding disclosed herein that especially suitable combinations of improved reaction buffer systems and alternative peptide tags can achieve particularly high DARs as shown in the examples provided herein.

These novel enzyme-based conjugation methods provide improved antibody-drug conjugates, whereby various antibodies can be used in the inventive methods. The inventive methods can be used for example to provide immunoconjugates (also referred to as antibody-drug conjugates (ADC) herein) comprising monoclonal antibodies directed against CEACAM5; these immunoconjugates have a cytotoxic effect, killing tumor cells in vitro and inhibiting tumor growth in vivo. However, application of the present invention is not limited to anti-CEACAM5 antibodies; rather, the present invention is applicable for the purposes of preparing antibody-linker conjugates or antibody-drug conjugates based on an antibody directed against any target. The present invention relates to embodiments described in the claims as well as in the further description herein below.

Aspects of the present invention relate to antibody modifications and payload conjugation strategies which significantly reduce the off-target cellular catabolism of such ADCs, thereby reducing the levels of released payload while improving the efficacy driven by higher ADC exposure. Therefore, these modifications will provide drugs with an improved therapeutic window by reduction of side effects and increase of antitumor activity.

As described herein, the exposure and half-life of the ADCs according to the invention will be improved for example by modification of the antibody heavy-chain with YTE mutations and/or using an innovative enzymatic conjugation method that uses a microbial transglutaminase (herein also referred to as “TGase” and “mTG”) enzyme to attach the drug to the antibody

Surprisingly, high exposure leading to low clearance values in human predictive animal models have been obtained by an approach as above. With such modifications the exposure of ADCs has been improved by ca. factor 10× compared to variants not comprising these modifications, which consequently results in a significant reduction of released payload levels and a significant increase of antitumor active ADC concentrations in circulation. The improved ADCs resembled the PK profile of the original YTE antibodies not comprising a drug linker and demonstrate an improved half-life over prior art ADCs.

These results will reduce toxicities and improve potencies in clinical cancer therapy compared to prior art molecules.

### BRIEF DESCRIPTION OF THE DRAWINGS

FIG. 1: Typical SEC chromatograms showing the purity of the input mAb and the final BDS for ADC7-M, ADC2-M and ADC5-M

FIG. 2: Typical RP-HPLC chromatograms illustrating DAR determination of the final BDS for ADC7-M, ADC2-M and ADC5-M

FIG. 3: In vitro potency of ADC2-M, ADC5-M and free payload against antigen-positive SK-CO-1 (FIG. 3a), SNU-16 (FIG. 3b), MKN-45 (FIG. 3c) and LS174T (FIG. 3d) cell lines in comparison to antigen-negative MDA-MB-231 (FIG. 3e) cell line. One representative experiment is shown, mean of duplicates+SD.

FIG. 4 Pharmacokinetic profile (total antibody) in huFcRn Tg276 mice for ADC1, ADC1-M, ADC2-M, ADC6-M and ADC7-M.

FIG. 5 Tumor volume changes after treatment with ADC1-M and ADC2-M versus vehicle control.

FIG. 6 More potent bystander effect of ADC1-M, ADC2-M, ADC6-M and ADC7-M compared with ADC SAR DM4 on antigen-negative MDA-MB-231 cells in co-culture with antigen-positive SK-CO-1 cells. One representative experiment is shown, mean of duplicates ±SD.

FIG. 7 Efficacy of ADC1-M and ADC3-M compared to ADC8 in an HPAF-II xenograft model.

FIG. 8 (A) Scheme of conjugation of small molecular drug/molecules to native, fully glycosylated mAbs. (B) Exemplary drug-linkers used as mTG substrates: Gly3-valine-citrulline-MMAE (“4M”= “drug-linker 4M”) and Gly3-beta-glucuronide exatecan (“1M”, also referred to herein as “drug-linker 1M” or “drug-linker compound 1-M”, “compound DL1-M” or “DL1-M”.

FIG. 9 (A) Conjugation of drug-linker 4M to native mAb trastuzumab using MTG from S. mobaraensis. 5 mg/mL antibody, 20 molar equivalents of drug-linker and varying mTG (purchased from Zedira, Germany) concentrations were mixed in 150 mM NaCl, 25 mM Tris-HCl, pH 8.0 with 10% DMSO. The amount of enzyme was titrated from 0 to 75 U/ml. Reactions were incubated for 16 h, stopped with MTG blocker (Zedira) followed by DAR determination using RP-HPLC as described in section 11.3.5. A DAR of 2 could not be reached even at high enzyme concentrations when using this prior art buffer (i.a. described in Strop, et al.; Chemistry & Biology (2013). 20 (2), 161-167 or Dickgiesser, et al.; Bioconjugate Chemistry 2020 31 (4), 1070-1076): 150 mM NaCl, 25 mM Tris-HCl, pH 8.0. (B) Conjugation of drug-linker 4M to native mAb using mTG from S. ladakanum (mTG variant 1, i.e. MTG_1 according to SEQ ID NO: 45) and the same buffer (150 mM NaCl, 25 mM Tris-HCl, pH 8.0) as in (A) or published by Dickgiesser et al. (2020). Reaction was conducted as described 11.3.4, the amount of enzyme was titrated in the range of 2.5-20 U/ml. Conjugation efficiency was higher with this mTG variant but a DAR of 2 was still not reached with this prior art buffer.

FIG. 10 Scheme of high throughput HTRF (homogeneous time-resolved fluorescence) procedure: Cadaverine-biotin is conjugated to native trastuzumab using mTG (A). Then the conjugated antibody is incubated with commercially available Streptavidin-d2 and anti-lgG antibodies labeled with Terbium (B). Once d2 and Terbium are in close proximity, a HTRF signal can be read out (C).

FIG. 11 (A) Impact of pH variation on transglutaminase-mediated bioconjugation over a range of buffer systems as indicated in the legend; (B) Impact of salt concentration (mM NaCl) on transglutaminase conjugation efficiency for HEPES and Tris-HCl buffer systems; (C) Impact of buffer substance HEPES concentration on transglutaminase conjugation efficiency.

FIG. 12 (A) Conjugation of drug-linker 4M to native fully glycosylated mAbs using S. ladakanum mTG in different buffer systems obtained from high-throughput screening. Buffers as indicated, control buffer is 25 mM Tris-HCl, 150 mM NaCl, pH 8.0. The improved buffer comprising 24 mM HEPES at pH 7.5 performed best. (B) Comparison of conjugation between improved buffer and prior art (150 mM NaCl, 25 mM Tris-HCl pH 8.0) buffer upon titration of mTG. Efficient conjugation of drug-linker 4M in the improved buffer can be achieved at only 10 U/ml mTG, while achieving a DAR=2 using S. ladakanum mTG in the prior art buffer typically requires much more mTG enzyme, namely about 70 U/ml.

FIG. 13 HIC-HPLC analysis of a monoclonal antibody that comprises the amino acid sequence “TLQS” within the light chains of the antibody. The corresponding ADC is shown.

FIG. 14 Mass spectrometry analysis of a monoclonal antibody that comprises the amino acid sequence “TLQS” within the light chains of the antibody and the corresponding ADC.

FIG. 15 HIC-HPLC analysis of tag fused antibodies mAb_tag2, mAb_tag3 and mAb_tag4 and the corresponding ADCs.

FIG. 16 Scheme of conjugation of mAbs with glutamine tags fused to the light chain C-termini using native mAb conjugation technology resulting in ADCs with DAR=4.

FIG. 17 Reduced RP-HPLC showing efficient conjugation to both the light chain (equipped with peptide tag) as well as the heavy chain (native glutamine). (A) Conjugation of drug-linker 4M to tag fused antibody mAb_tag 5 resulting in DAR 3.9. (B) Conjugation of drug-linker 1M to tag fused antibody mAb_tag5 with a DAR of 3.9. (C) Conjugation of drug-linker 1M to mAb7-M′ (top) resulting in ADC7-M′ bottom; DAR of 3.9).

FIG. 18 pH dependency of transglutaminase-mediated bioconjugation over a range of buffer systems.

### DETAILED DESCRIPTION OF THE INVENTION

### Definitions

As used herein “CEACAM5” designates the “carcino-embryonic antigen-related cell adhesion molecule 5”, also known as “CD66e” (Cluster of Differentiation 66e). CEACAM5 is a glycoprotein involved in cell adhesion. CEACAM5 is highly expressed especially on the surface of e.g. colorectal cancer, gastric cancer, non-small cell lung cancer, pancreatic cancer, esophageal cancer, prostate cancer and other solid tumors.

A “domain” or “region” may be any region of a protein, generally defined on the basis of sequence homologies and often related to a specific structural or functional entity.

A “coding sequence” or a sequence “encoding” an expression product, such as a polypeptide, protein, or enzyme, is a nucleotide sequence that, when expressed, results in the production of that polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, references to specific proteins (e.g. antibodies) can include a polypeptide having a native amino acid sequence, as well as variants and modified forms regardless of their origin or mode of preparation. A protein which has a native amino acid sequence is a protein having the same amino acid sequence as obtained from nature. Such native sequence proteins can be isolated from nature or can be prepared using standard recombinant and/or synthetic methods. Native sequence proteins specifically encompass naturally occurring truncated or soluble forms, naturally occurring variant forms (e.g. alternatively spliced forms), naturally occurring allelic variants and forms including post-translational modifications. Native sequence proteins include proteins carrying post-translational modifications such as glycosylation, or phosphorylation, or other modifications of some amino acid residues.

The term “gene” means a DNA sequence that codes for, or corresponds to, a particular sequence of amino acids which comprises all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

Herein, a sequence “at least 85% identical” to a reference sequence is a sequence having, over its entire length, 85% or more, for instance 90%, 91%, 92%, 93%, 94%, 95%, 96%, 97%, 98% or 99% sequence identity with the entire length of the reference sequence. The percentage of “sequence identity” may thus be determined by comparing two such sequences over their entire length by global pairwise alignment using the algorithm of Needleman and Wunsch (J. Mol. Biol. 48:443 (1970)), e.g. using the program Needle (EMBOSS) with the BLOSUM62 matrix and the following parameters: gap open=10, gap extend=0.5, end gap penalty=false, end gap open=10, end gap extend=0.5 (which are standard settings). A “conservative amino acid substitution” is one in which an amino acid residue is substituted by another amino acid residue having a side chain with similar chemical properties (e.g., charge, size or hydrophobicity). In general, a conservative amino acid substitution will not substantially change the functional properties of a protein. Examples of groups of amino acids that have side chains with similar chemical properties include 1) aliphatic side chains: glycine, alanine, valine, leucine, and isoleucine; 2) aliphatic-hydroxyl side chains: serine and threonine; 3) amide-containing side chains: asparagine and glutamine; 4) aromatic side chains:

phenylalanine, tyrosine, and tryptophan; 5) basic side chains: lysine, arginine, and histidine; 6) acidic side chains: aspartic acid and glutamic acid; and 7) sulfur-containing side chains:

cysteine and methionine. Conservative amino acid substitution groups can also be defined on the basis of amino acid size.

An “antibody” (also referred to as an “immunoglobulin”) may e.g. be a natural or conventional type of antibody in which two heavy chains are linked to each other by disulfide bonds and each heavy chain is linked to a light chain by a disulfide bond. There are two types of light chain, lambda (I) and kappa (κ). There are five main heavy chain classes (or isotypes) which determine aspects of the functional activity of an antibody molecule: IgM, IgD, IgG, IgA and IgE. Each antibody chain contains distinct sequence domains (or regions). The light chain of a typical lgG antibody includes two regions, a variable region (VL) and a constant region (CL).

The heavy chain of a typical IgG antibody includes four regions, namely a variable region (VH) and a constant region (CH), the latter being made up of three constant domains (CH1, CH2 and CH3). The variable regions of both light and heavy chains determine binding and specificity to the antigen. The constant regions of the light and heavy chains can confer important biological properties, such as antibody chain association, secretion, trans-placental mobility, complement binding, and binding to Fc receptors (FcR). The Fv fragment is the N-terminal part of the Fab fragment of an antibody and consists of the variable portions of one light chain and one heavy chain.

The specificity of the antibody resides in the structural complementarity between the antibody combining site and the antigenic determinant. Antibody combining sites are made up of residues that are primarily from the so-called hypervariable or complementarity determining regions (CDRs). Complementarity determining regions (CDRs) therefore refer to amino acid sequences which together define the binding affinity and specificity of the Fv region of an antibody. The light (L) and heavy (H) chains of an antibody each have three CDRs, designated CDR1-L, CDR2-L, CDR3-L and CDR1-H, CDR2-H, CDR3-H, respectively. A conventional antibody's antigen-binding site, therefore, includes six CDRs, comprising the CDR set from each of a heavy and a light chain variable region.

“Framework regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR1-L, FR2-L, FR3-L, FR4-L, and FR1-H, FR2-H, FR3-H, FR4-H, respectively. As used herein, a “human framework region” is a framework region that is substantially identical (about 85%, or more, for instance 90%, 91%, 92%, 93%, 94%, 95%, 96%, 97%, 98% or 99%) to the framework region of a naturally occurring human antibody.

In the context of the invention, CDR/FR definition in an immunoglobulin light or heavy chain is determined based on the IMGT definition (Lefranc et al. Dev. Comp. Immunol., 2003, 27 (1): 55-77; www.imgt.org).

As used herein, the term “antibody” includes conventional antibodies and fragments thereof, as well as single domain antibodies and fragments thereof, such as variable heavy chain of single domain antibodies; the term “antibody” as used herein also includes chimeric, humanized, bispecific or multispecific antibodies, as well as other types of engineered antibodies. The term “antibody” includes monoclonal antibodies.

The term “monoclonal antibody” or “mAb” as used herein refers to an antibody molecule of a single amino acid sequence, which is directed against a specific antigen, and is not to be construed as requiring production of the antibody by any particular method. A monoclonal antibody may be produced e.g. by a single clone of B cells or hybridoma, but may also be recombinant, e.g. produced by methods involving genetic or protein engineering.

The term “chimeric antibody” refers to an engineered antibody which, in its broadest sense, contains one or more regions from one antibody and one or more regions from one or more other antibodies. In an embodiment, a chimeric antibody comprises a VH and a VL of an antibody derived from a non-human animal, in association with a CH and a CL of another antibody which is, in some embodiments, a human antibody. As the non-human animal, any animal such as mouse, rat, hamster, rabbit or the like can be used. A chimeric antibody may also denote a multispecific antibody having specificity for at least two different antigens.

The term “humanized antibody” refers to an antibody which is wholly or partially of non-human origin and which has been modified to replace certain amino acids, for instance in the framework regions of the VH and VL, in order to avoid or minimize an immune response in humans. The constant regions of a humanized antibody are typically human CH and CL regions.

“Fragments” of antibodies (e.g. of conventional antibodies) comprise a portion of an intact antibody such as an lgG, in particular an antigen binding region or variable region of the intact antibody. Examples of antibody fragments include Fv, Fab, F(ab′)2, Fab′, dsFv, (dsFv)2, scFv, sc (Fv)2, diabodies, as well as bispecific and multispecific antibodies formed from antibody fragments. A fragment of a conventional antibody may also be a single domain antibody, such as a heavy chain antibody or VHH.

The term “Fab” denotes an antibody fragment having a molecular weight of about 50,000 Da and antigen binding activity, in which about a half of the N-terminal side of the heavy chain and the entire light chain are bound together through a disulfide bond. It is usually obtained among fragments by treating lgG with a protease, papaine.

The term “F(ab′)2” refers to an antibody fragment having a molecular weight of about 100,000 Da and antigen binding activity, which is slightly larger than 2 identical Fab fragments bound via a disulfide bond of the hinge region. It is usually obtained among fragments by treating lgG with a protease, pepsin.

The term “Fab′” refers to an antibody fragment having a molecular weight of about 50,000 Da and antigen binding activity, which is obtained by cutting a disulfide bond of the hinge region of the F (ab′) 2.

A single chain Fv (“scFv”) is a covalently linked VH: VL heterodimer which is usually expressed from a gene fusion including VH and VL encoding genes linked by a peptide-encoding linker.

The human scFv fragments of the invention include CDRs that are held in appropriate conformation, for instance by using gene recombination techniques. Divalent and multivalent antibody fragments can form either spontaneously by association of monovalent scFvs, or can be generated by coupling monovalent scFvs by a peptide linker, such as divalent sc (Fv)2. “dsFv” is a VH::VL heterodimer stabilised by a disulphide bond. “(dsFv)2” denotes two dsFv coupled by a peptide linker.

The term “bispecific antibody” or “BsAb” denotes an antibody which comprises two different antigen binding sites. Thus, BsAbs are able to e.g. bind two different antigens simultaneously. Genetic engineering has been used with increasing frequency to design, modify, and produce antibodies or antibody derivatives with a desired set of binding properties and effector functions as described for instance in EP 2 050 764 A1.

The term “multispecific antibody” denotes an antibody which comprises two or more different antigen binding sites.

The term “diabodies” refers to small antibody fragments with two antigen binding sites, which fragments comprise a heavy-chain variable domain (VH) connected to a light-chain variable domain (VL) in the same polypeptide chain (VH-VL). By using a linker that is too short to allow pairing between the two domains of the same chain, the domains are forced to pair with the complementary domains of another chain and create two antigen-binding sites.

The term “hybridoma” denotes a cell, which is obtained by subjecting a B cell prepared by immunizing a non-human mammal with an antigen to cell fusion with a myeloma cell derived from a mouse or the like which produces a desired monoclonal antibody having an antigen specificity.

By “purified” or “isolated” it is meant, when referring to a polypeptide (e.g. an antibody) or a nucleotide sequence, that the indicated molecule is present in the substantial absence of other biological macromolecules of the same type. The term “purified” as used herein means at least 75%, 85%, 95%, 96%, 97%, or 98% by weight, of biological macromolecules of the same type are present. An “isolated” nucleic acid molecule which encodes a particular polypeptide refers to a nucleic acid molecule which is substantially free of other nucleic acid molecules that do not encode the subject polypeptide; however, the molecule may include some additional bases or moieties which do not deleteriously affect the basic characteristics of the composition.

As used herein, the term “subject” denotes a mammal, such as a rodent, a feline, a canine, a primate or a human. In embodiments of the invention, the subject (or patient) is a human.

As used herein the term “about” in connection with a numerical value means that the value can vary between +/−10% of said value.

As used here in unless otherwise specified the concentration of DMSO is given in % (v/v).

### Antibodies

Any antibody can be used in the conjugation methods of this invention. Preferred antibodies include monoclonal antibodies that comprises an amino acid sequence selected from the group consisting of GGTLQSPP (SEQ ID NO: 74), TLQSG (SEQ ID NO: 77), TLQSPP (SEQ ID NO: 78), GGTLQSG (SEQ ID NO: 79) and TLQSA (SEQ ID NO: 80) and more preferably the amino acid sequence TLQSPP (SEQ ID NO: 78) or GGTLQSPP (SEQ ID NO: 74) (most preferably the amino acid sequence GGTLQSPP) in at least one and preferably both of its light chain constant regions (CL) and/or in at least one and preferably both of its heavy chain constant regions (CH); and/or comprises a glutamine at position 295 (EU numbering). These amino acid sequences have been found to be suitable to serve as substrate for the microbial transglutaminase (herein also “mTG” or “MTG”), even when incorporated into an antibody light chain constant regions (CL) and/or heavy chain constant regions (CH). These amino acid sequences are therefore also designated herein as “mTG tags”. When included in an antibody's CL and/or CH chain (preferably at the C-terminus of said chain), then the primary amino group of a linker (preferably a drug linker; but the linker can also include other payloads besides drugs, such as a detectable label or a second antibody) or the primary amino group of a therapeutic agent can react with the glutamine of these aforementioned amino acid sequences of the antibody in the presence of the mTG enzyme:

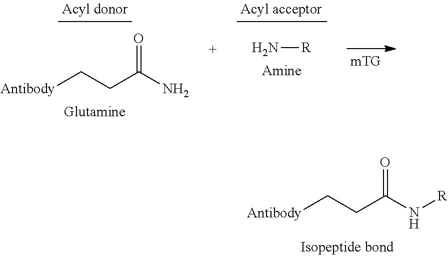

In the above shown reaction scheme the acyl acceptor is said linker (preferably a drug-linker) or said therapeutic agent that comprises said primary amino group.

The inventors have previously generated, screened and selected specific anti-CEACAM5 antibodies surprisingly displaying a combination of several characteristics that make them ideally suited for use in cancer therapy, in particular as part of an immunoconjugate (antibody-drug conjugate). Also these antibodies can be used in a conjugation method of the invention.

Some of these antibodies have been used as examples for the present invention, e.g. for conjugation by the methods of the invention. However, as mentioned, the application of the present invention is not limited to anti-CEACAM5 antibodies; rather, the present invention is applicable for the purposes of preparing antibody-linker conjugates or antibody-drug conjugates based on an antibody directed against any target.

Preferred antibodies that can be used in the method of the present invention include an isolated antibody (e.g. one which binds to human CEACAM5 protein);

and wherein the isolated antibody comprises
(i) at least one light chain constant region (CL) that comprises a sequence selected from the group consisting of GGTLQSPP, TLQSG, TLQSPP and TLQSA (preferably GGTLQSPP) and preferably comprising this sequence at the C-terminus of said light chain constant region; and/or
(ii) at least one heavy chain constant region (CH) comprising one or more of the following amino acid substitutions:
   (a) L234A and L235A (LALA mutation);
   (b) L234A and L235A and P329G (LALA-PG mutation);
   (c) L235A and G237A (LAGA mutation);
   (d) M252Y and S254T and T256E (YTE mutation);
   (e) K222R;
      and wherein Eu numbering is used for said amino acid substitutions.

Further preferred antibodies that can be used in the methods of the invention include an isolated antibody which binds to human CEACAM5 protein and wherein the isolated antibody comprises
(i) at least one light chain constant region (CL) that comprises a sequence selected from the group consisting of GGTLQSPP, TLQSG, TLQSPP and TLQSA (preferably GGTLQSPP) and preferably comprising this sequence at the C-terminus of said light chain constant region;
and
(ii) at least one heavy chain constant region (CH) comprising one or more of the following amino acid substitutions:
   (a) L234A and L235A (LALA mutation);
   (b) L234A and L235A and P329G (LALA-PG mutation);
   (c) L235A and G237A (LAGA mutation);
   (d) M252Y and S254T and T256E (YTE mutation);
   (e) K222R;

and wherein Eu numbering is used for said amino acid substitutions;

and wherein said isolated antibody comprises a CDR1-H consisting of the amino acid sequence of SEQ ID NO: 3, a CDR2-H consisting of the amino acid sequence of SEQ ID NO: 4, a CDR3-H consisting of the amino acid sequence of SEQ ID NO: 5, a CDR1-L consisting of the amino acid sequence of SEQ ID NO: 6, a CDR2-L consisting of the amino acid sequence of SEQ ID NO: 7, and a CDR3-L consisting of the amino acid sequence of SEQ ID NO: 8.

Preferably, an isolated antibody that can be used in a method of the invention comprises framework regions FR1, FR2, FR3, FR4, FR5, FR6, FR7 and FR8 having the structure FR1-CDR1-H-FR2-CDR2-H-FR3-CDR3-H-FR4 and FR5-CDR1-L-FR6-CDR2-L-FR7 CDR3-L-FR8; wherein FR1 consists of SEQ ID NO: 54, FR2 consists of SEQ ID NO: 55, FR3 consists of SEQ ID NO: 56, FR4 consists of SEQ ID NO: 57, FR5 consists of SEQ ID NO: 58, FR6 consists of SEQ ID NO: 59, FR7 consists of SEQ ID NO: 60 and FR8 consists of SEQ ID NO: 61.

The present invention also provides a conjugation method as described herein that uses an isolated antibody (e.g. one which binds to human CEACAM5 protein) wherein the isolated antibody comprises
(i) at least one heavy chain constant region (CH) comprising one or more of the following amino acid substitutions:
   (a) L234A and L235A (LALA mutation);
   (b) L234A and L235A and P329G (LALA-PG mutation);
   (c) L235A and G237A (LAGA mutation);
   (d) M252Y and S254T and T256E (YTE mutation);
   (e) K222R;
and
(ii) at least one light chain constant region (CL) that comprises the sequence GGTLQSPP, and preferably comprising this sequence at the C-terminus of said light chain constant region;

and wherein Eu numbering is used for said amino acid substitutions. The Eu numbering system is well known (cf. Edelman et al., Proc. Natl. Acad. Sci. USA 1969, 63, 78-85 and Kabat, E. A. et al., National Institutes of Health (U.S.) Office of the Director. Sequences of Proteins of Immunological Interest, 5th ed.; DIANE Publishing: Collingdale, PA, USA, 1991) and the positions of the amino acid substitutions that are indicated follow this numbering system. The amino acid substitutions are specified using the single letter amino acid code. The GGTLQSPP can also be comprised in the light chain constant region (CL) several times and can alternatively or additionally also be comprised in the heavy chain constant region (CH). Preferably, the GGTLQSPP is comprised once per light chain constant region (CL) in both light chain constant regions (CL) of the antibody of the invention.

In an embodiment of the isolated antibody that can be used in the conjugation methods of the invention, both heavy chain constant regions (CH) comprise one or more of said amino acid substitutions (a) through (e) and/or wherein both light chain constant regions comprise said sequence GGTLQSPP. Preferred combinations of modifications of the CL and CH chains are outlined in Table 4 below that indicates the modification combinations for antibodies mAb1-M, mAb2-M, mAb3-M, mAb6-M and mAb7-M. Preferably, the antibody of the invention comprises any of the following heavy chain constant region (CH) and light chain constant regions (CL) modifications:
(a) the CH comprises the amino acid substitutions L234A, L235A (LALA mutation) and M252Y, S254T and T256E (YTE mutation); or
(b) the CH comprises the amino acid substitutions L234A, L235A (LALA mutation) and M252Y, S254T and T256E (YTE mutation); and the light chain constant region (CL) that comprises the sequence GGTLQSPP (preferably at the C-terminus); or
(c) the CH comprises the amino acid substitutions L234A, L235A (LALA mutation) and M252Y, S254T and T256E (YTE mutation) and K222R; and the light chain constant region (CL) that comprises the sequence GGTLQSPP (preferably at the C-terminus); or
(d) the CH comprises the amino acid substitutions L234A, L235A (LALA mutation); or
(e) the CH comprises the amino acid substitutions L234A, L235A (LALA mutation); and the light chain constant region (CL) that comprises the sequence GGTLQSPP (preferably at the C-terminus).

Preferably, both CL and both CH regions of the antibody of the invention comprise a modification as outlined in (a) through (e) above.

Any combination of the embodiments described herein above and below forms part of the invention.

In some embodiments of the conjugation method of the invention, the antibody that is used in the method of the invention is a conventional antibody, such as a conventional monoclonal antibody, or an antibody fragment, a bispecific or multispecific antibody.

In some embodiments, the antibody comprises or consists of an lgG, or a fragment thereof.

In some embodiments, the antibody that is used in the conjugation method of the invention may be e.g. a murine antibody, a chimeric antibody, a humanized antibody, or a human antibody. Numerous methods for humanization of an antibody sequence are known in the art; see e.g. the review by Almagro & Fransson (2008) Front Biosci. 13:1619-1633. One commonly used method is CDR grafting, or antibody reshaping, which involves grafting of the CDR sequences of a donor antibody, generally a mouse antibody, into the framework scaffold of a human antibody of different specificity. Since CDR grafting may reduce the binding specificity and affinity, and thus the biological activity, of a CDR grafted non-human antibody, back mutations may be introduced at selected positions of the CDR grafted antibody in order to retain the binding specificity and affinity of the parent antibody. Identification of positions for possible back mutations can be performed using information available in the literature and in antibody databases. Amino acid residues that are candidates for back mutations are typically those that are located at the surface of an antibody molecule, while residues that are buried or that have a low degree of surface exposure will not normally be altered. An alternative humanization technique to CDR grafting and back mutation is resurfacing, in which non-surface exposed residues of non-human origin are retained, while surface residues are altered to human residues. Another alternative technique is known as “guided selection” (Jespers et al. (1994) Biotechnology 12, 899) and can be used to derive from a murine antibody a fully human antibody conserving the epitope and binding charateristics of the parental antibody.

For chimeric antibodies, humanization typically involves modification of the framework regions of the variable region sequences.

Amino acid residues that are part of a CDR will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, for instance by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, for instance Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the present invention.

In a humanized antibody or fragment thereof, the variable domains of heavy and light chains may comprise human acceptor framework regions. A humanized antibody may further comprise human constant heavy and light chain domains, where present.

In some embodiments, the antibody used in the conjugation methods of the invention may be an antibody fragment (for instance a humanized antibody fragment) selected from the group consisting of Fv, Fab, F(ab′)2, Fab′, dsFv, (dsFv)2, scFv, sc (Fv)2, and diabodies.

In some embodiments, the antibody may be a bispecific or multispecific antibody formed from antibody fragments, at least one antibody fragment being a fragment of an antibody according to the present invention. Multispecific antibodies are polyvalent protein complexes as described for instance in EP 2 050 764 A1 or US 2005/0003403 A1.

The antibodies useful in the conjugation methods of the invention can be produced by any technique known in the art. Antibodies according to the invention can be used e.g. in an isolated (e.g. purified) form or contained in a vector, such as a membrane or lipid vesicle (e.g. a liposome).

If an antibody is used in the conjugation method of the invention that is not an lgG antibody then it is preferred that that antibody comprises an amino acid sequence selected from the group consisting of GGTLQSPP, TLQSG, TLQSPP, GGTLQSG and TLQSA.

### Nucleic Acids and Host Cells

A further aspect of the disclosure relates to an isolated nucleic acid comprising or consisting of a nucleic acid sequence encoding an antibody of the invention as defined above.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

Accordingly, a further aspect of the disclosure relates to a vector comprising a nucleic acid as defined above.

Such vectors may comprise regulatory elements, such as a promoter, enhancer, terminator and the like, to cause or direct expression of said polypeptide upon administration to a subject. Examples of promoters and enhancers used in the expression vector for an animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cells can be used, so long as a gene encoding the human antibody C region can be inserted and expressed. Examples of suitable vectors include PAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like.

Other examples of plasmids include replicating plasmids comprising an origin of replication, or integrative plasmids, such as for instance pUC, pcDNA, pBR, and the like.

Other examples of viral vectors include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present disclosure relates to a host cell which has been transfected, infected or transformed by a nucleic acid and/or a vector.

The term “transformation” means the introduction of a “foreign” (i.e. extrinsic) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids may be used to produce an antibody of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like. In some embodiments, the YB2/0 cell is used, since ADCC activity of chimeric or humanized antibodies is enhanced when expressed in this cell.

For expression of a humanized antibody, the expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, a humanized antibody expression vector is of the tandem type shitara K et al. J Immunol Methods. 1994 Jan. 3;167 (1-2): 271-8). Examples of tandem type humanized antibody expression vector include pKANTEX93 (WO 97/10354), pEE18 and the like.

The present dislcosure also relates to a method of producing a recombinant host cell expressing an antibody according to the invention, said method comprising the steps consisting of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody.

Such recombinant host cells can be used for the production of antibodies of the invention.

### Methods of Producing Antibodies

Antibodies that can be used for conjugation with a linker or drug linker using the methods of the invention may be obtained or produced by any technique known in the art, such as, without limitation, any chemical, biological, genetic or enzymatic technique, either alone or in combination.

Knowing the amino acid sequence of a desired antibody, one skilled in the art can readily produce said antibodies or immunoglobulin chains using standard techniques for production of polypeptides. For instance, they can be synthesized using well-known solid phase methods using a commercially available peptide synthesis apparatus (such as that made by Applied Biosystems, Foster City, California) and following the manufacturer's instructions. Alternatively, antibodies and immunoglobulin chains of the invention can be produced by recombinant DNA techniques, as is well-known in the art. For example, these polypeptides (e.g. antibodies) can be obtained as DNA expression products after incorporation of DNA sequences encoding the desired polypeptide into expression vectors and introduction of such vectors into suitable eukaryotic or prokaryotic hosts that will express the desired polypeptide, from which they can be later isolated using well-known techniques.

The invention further relates to a method of producing an antibody of the invention, which method comprises the steps consisting of: (i) culturing a transformed host cell; (ii) expressing the antibody; and (iii) recovering the expressed antibody.

Antibodies of the invention can be suitably separated from the culture medium by conventional immunoglobulin purification procedures such as, for example, protein A-Sepharose, hydroxyapatite chromatography, gel electrophoresis, dialysis, or affinity chromatography.

In some embodiments, a humanized chimeric antibody of the present invention can be produced by obtaining nucleic acid sequences encoding humanized VL and VH regions as previously described, constructing a human chimeric antibody expression vector by inserting them into an expression vector for animal cell having genes encoding human antibody CH and human antibody CL, and expressing the coding sequence by introducing the expression vector into an animal cell.

As the CH domain of a human chimeric antibody, any region which belongs to human immunoglobulin heavy chains may be used, for instance those of IgG class are suitable and any one of subclasses belonging to lgG class, such as IgG1, IgG2, IgG3 and IgG4, can be used. Also, as the CL of a human chimeric antibody, any region which belongs to human immunoglobulin light chains may be used, and those of kappa class or lambda class can be used.

Methods for producing humanized or chimeric antibodies may involve conventional recombinant DNA and gene transfection techniques are well known in the art (see e.g. Morrison SL. et al. (1984) and patent documents U.S. Pat. Nos. 5,202,238, 5,204,244).

Methods for producing humanized antibodies based on conventional recombinant DNA and gene transfection techniques are well known in the art (see, e. g., Riechmann L. et al. 1988; Neuberger MS. et al. 1985). Antibodies can be humanized using a variety of techniques known in the art including, for example, the technique disclosed in the application WO2009/032661, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan EA (1991); Studnicka G M et al. (1994); Roguska MA. et al. (1994)), and chain shuffling (U.S. Pat. No. 5,565,332). The general recombinant DNA technology for preparation of such antibodies is also known (see European Patent Application EP 125023 and International Patent Application WO 96/02576).

A Fab of the present invention can be obtained by treating an antibody of the invention (e.g. an lgG) with a protease, such as papaine. Also, the Fab can be produced by inserting DNA sequences encoding both chains of the Fab of the antibody into a vector for prokaryotic expression, or for eukaryotic expression, and introducing the vector into prokaryotic or eukaryotic cells (as appropriate) to express the Fab.

A F (ab′) 2 of the present invention can be obtained treating an antibody of the invention (e.g. an lgG) with a protease, pepsin. Also, the F (ab′) 2 can be produced by binding a Fab′ described below via a thioether bond or a disulfide bond.

A Fab′ of the present invention can be obtained by treating F (ab′) 2 of the invention with a reducing agent, such as dithiothreitol. Also, the Fab′ can be produced by inserting DNA sequences encoding Fab′ chains of the antibody into a vector for prokaryotic expression, or a vector for eukaryotic expression, and introducing the vector into prokaryotic or eukaryotic cells (as appropriate) to perform its expression.

A scFv of the present invention can be produced by taking sequences of the CDRs or VH and VL domains as previously described for the antibody of the invention, then constructing a DNA encoding a scFv fragment, inserting the DNA into a prokaryotic or eukaryotic expression vector, and then introducing the expression vector into prokaryotic or eukaryotic cells (as appropriate) to express the scFv. To generate a humanized scFv fragment, a well-known technology called CDR grafting may be used, which involves selecting the complementary determining regions (CDRs) according to the invention, and grafting them onto a human scFv fragment framework of known three dimensional structure (see, e. g., WO98/45322; WO 87/02671; U.S. Pat. Nos. 5,859,205; 5,585,089; 4,816,567; EP0173494).

### Modification of the Antibodies of the Invention

Amino acid sequence modification(s) of the antibodies described herein are contemplated. For example, it may be desirable to improve the binding affinity and/or other biological properties of the antibody.

Modifications and changes may be made in the structure of the antibodies of the present invention, and in the DNA sequences encoding them, and still result in a functional antibody or polypeptide with desirable characteristics.

In making the changes in the amino sequences of polypeptide, the hydropathic index of amino acids may be considered. The importance of the hydropathic amino acid index for the interactive biologic function of a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further aspect of the present invention also encompasses function-conservative variants of the polypeptides of the present invention.

For example, certain amino acids may be substituted by other amino acids in a protein structure without appreciable loss of activity. Since the interactive capacity and nature of a protein define its biological functional activity, certain amino acid substitutions can be made in a protein sequence, and of course in its encoding DNA sequence, while nevertheless obtaining a protein with like properties. It is thus contemplated that various changes may be made in the antibody sequences of the invention, or corresponding DNA sequences which encode said polypeptides, without appreciable loss of their biological activity.

It is known in the art that certain amino acids may be substituted by other amino acids having a similar hydropathic index or score and still result in a protein with similar biological activity, i.e. still obtain a biological functionally equivalent protein. It is also possible to use well-established technologies, such as alanine-scanning approaches, to identify, in an antibody or polypeptide of the invention, all the amino acids that can be substituted without significant loss of binding to the antigen. Such residues can be qualified as neutral, since they are not involved in antigen binding or in maintaining the structure of the antibody. One or more of these neutral positions can be substituted by alanine or by another amino acid can without changing the main characteristics of the antibody or polypeptide of the invention.

Neutral positions can be seen as positions where any amino acid substitution could be incorporated. Indeed, in the principle of alanine-scanning, alanine is chosen since it this residue does not carry specific structural or chemical features. It is generally admitted that if an alanine can be substituted for a specific amino acid without changing the properties of a protein, many other, if not all amino acid substitutions are likely to be also neutral. In the opposite case where alanine is the wild-type amino acid, if a specific substitution can be shown as neutral, it is likely that other substitutions would also be neutral.

As outlined above, amino acid substitutions are generally based on the relative similarity of the amino acid side-chain substituents, for example, their hydrophobicity, hydrophilicity, charge, size, and the like. Exemplary substitutions which take any of the foregoing characteristics into consideration are well known to those of skill in the art and include: arginine and lysine; glutamate and aspartate; serine and threonine; glutamine and asparagine; and valine, leucine and isoleucine.

It may be also desirable to modify the antibody of the invention with respect to effector function, e.g. so as to enhance antigen-dependent cell-mediated cytotoxicity (ADCC) and/or complement dependent cytotoxicity (CDC) of the antibody, or e.g. to alter the binding to Fc receptors. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity

(ADCC) (Caron PC. et al. 1992; and Shopes B. 1992). In some embodiments, an antibody of the invention may be an antibody with a modified amino acid sequence that results in reduced or eliminated binding to most Fcγ receptors, which can reduce uptake and toxicity in normal cells and tissues expressing such receptors, e.g. macrophages, liver sinusoidal cells etc., An example for such an antibody is one including substitutions of two leucine (L) residues to alanine (A) at position 234 and 235 (i.e. LALA); this double substitution has been demonstrated to reduce Fc binding to FcγRs and consequently to decrease ADCC as well to reduce complement binding/activation. Another example for such an antibody is one including the substitution P329G in addition to the LALA double substitution (i.e. PG-LALA; see e.g. Schlothauer et al., Novel human IgG1 and IgG4 Fc-engineered antibodies with completely abolished immune effector functions, Protein Engineering, Design and Selection, Volume 29, Issue 10, October 2016, Pages 457-466). In some embodiments, an antibody of the invention may thus be an antibody having an amino acid sequence that (i) contains e.g. the LALA or the PG-LALA set of substitutions and (ii) is otherwise identical to the amino acid sequence of one of the antibodies of the invention described herein above with reference to the respective SEQ ID NOs.

Another type of amino acid modification of the antibody of the invention may be useful for altering the original glycosylation pattern of the antibody, i.e. by deleting one or more carbohydrate moieties found in the antibody, and/or adding one or more glycosylation sites that are not present in the antibody. The presence of either of the tripeptide sequences asparagine-X-serine, and asparagine-X-threonine, where X is any amino acid except proline, creates a potential glycosylation site. Addition or deletion of glycosylation sites to the antibody can conveniently be accomplished by altering the amino acid sequence such that it contains one or more of the above-described tripeptide sequences (for N-linked glycosylation sites).

Another type of modification involves the removal of sequences identified, either in silico or experimentally, as potentially resulting in degradation products or heterogeneity of antibody preparations. As examples, deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in an antibody or polypeptide, it may therefore be considered to remove the site, typically by conservative substitution to remove one of the implicated residues. Such substitutions in a sequence to remove one or more of the implicated residues are also intended to be encompassed by the present invention.

Another type of covalent modification involves chemically or enzymatically coupling glycosides to the antibody. These procedures are advantageous in that they do not require production of the antibody in a host cell that has glycosylation capabilities for N- or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Removal of carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo- and exo-glycosidases as described by Thotakura, N R. et al. (1987).

Another type of covalent modification of the antibody comprises linking the antibody to one of a variety of non-proteinaceous polymers, e.g. polyethylene glycol, polypropylene glycol, or polyoxyalkylenes, e.g. in the manner set forth in U.S. Pat. Nos. 4,640,835; 4,496,689; 4,301,144; 4,670,417; 4,791,192 or 4,179,337.

Other amino acid sequence modifications known in the art may also be applied to an antibody of the invention.

### Immunoconjugates

The present invention provides immunoconjugates, also referred to herein as antibody-drug conjugates or, more briefly, conjugates. As used herein, all these terms have the same meaning and are interchangeable. Suitable methods for preparing immunoconjugates are known in the art. The immunoconjugates of the invention may be prepared by in vitro methods, e.g. as described herein; preferably they can be prepared by a method according to the present invention.

The present invention provides an immunoconjugate comprising an antibody of the invention covalently linked via a linker to at least one growth inhibitory agent or to at least one other agent. Other agents include a detectable label or therapeutic agent. A therapeutic agent preferably is a cytotoxic drug.

The term “growth inhibitory agent” (also referred to as an “anti-proliferative agent”) refers to a molecule or compound or composition which inhibits growth of a cell, such as a tumor cell, in vitro and/or in vivo.

In some embodiments, the growth inhibitory agent is a cytotoxic drug (also referred to as a cytotoxic agent). In some embodiments, the growth inhibitory agent is a radioactive moiety.

The term “cytotoxic drug” as used herein refers to a substance that directly or indirectly inhibits or prevents the function of cells and/or causes destruction of the cells. The term “cytotoxic drug” includes e.g. chemotherapeutic agents, enzymes, antibiotics, toxins such as small molecule toxins or enzymatically active toxins, toxoids, vincas, taxanes, maytansinoids or maytansinoid analogs, tomaymycin or pyrrolobenzodiazepine derivatives, cryptophycin derivatives, leptomycin derivatives, auristatin or dolastatin analogs, prodrugs, topoisomerase I inhibitors, topoisomerase II inhibitors, DNA alkylating agents, anti-tubulin agents, CC-1065 and CC-1065 analogs.

Topoisomerase I inhibitors are molecules or compounds that inhibit the human enzyme topoisomerase I which is involved in altering the topology of DNA by catalyzing the transient breaking and rejoining of a single strand of DNA. Topoisomerase I inhibitors are highly toxic to dividing cells e.g. of a mammal. Examples of suitable topoisomerase I inhibitors include camptothecin (CPT) and analogs thereof such as topotecan, irinotecan, silatecan, cositecan, exatecan, lurtotecan, gimatecan, belotecan and rubitecan.

In some embodiments, the immunoconjugates of the invention comprise the cytotoxic drug exatecan as the growth inhibitory agent. Exatecan has the chemical name (1S,9S)-1-Amino-9-ethyl-5-fluoro-1,2,3,9,12,15-hexahydro-9-hydroxy-4-methyl-10H, 13H-benzo (de) pyrano (3′,4′: 6,7) indolizino (1,2-b) quinoline-10,13-dione. Exatecan is represented by the following structural formula (I):

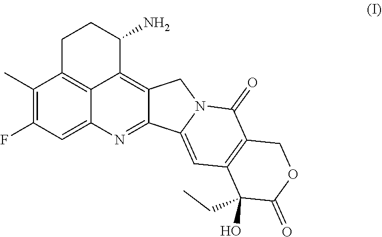 [SMILES: CC[C@@]1(O)C(=O)OCc2c1cc1n(c2=O)Cc2c-1nc1cc(F)c(C)c3c1c2[C@@H](N)CC3]

In further embodiments of the invention, other CPT analogs and other cytotoxic drugs may be used, e.g. as listed above. Examples of some cytotoxic drugs and of methods of conjugation are further given in the application WO2008/010101 which is incorporated by reference.

The term “radioactive moiety” refers to a chemical entity (such as a molecule, compound or composition) that comprises or consists of a radioactive isotope suitable for treating cancer, such as At²¹¹, Bi²¹², Er¹⁶⁹, I¹³¹, I¹²⁵, Y⁹⁰, In¹¹¹, P³², Re¹⁸⁶, Re¹⁸⁸, Sm¹⁵³, Sr⁸⁹, or radioactive isotopes of Lu. Such radioisotopes generally emit mainly beta-radiation. In some embodiments, the radioactive isotope is an alpha-emitter isotope, for example Thorium 227 which emits alpha-radiation. Immunoconjugates can be prepared e.g. as described in the application WO2004/091668.

In an immunoconjugate of the present invention, an antibody of the present invention is covalently linked via a linker to the at least one growth inhibitory agent. “Linker”, as used herein, means a chemical moiety comprising a covalent bond and/or any chain of atoms that covalently attaches the growth inhibitory agent to the antibody. Linkers are well known in the art and include e.g. disulfide groups, thioether groups, acid labile groups, photolabile groups, peptidase labile groups and esterase labile groups. Conjugation of an antibody of the invention with cytotoxic drugs or other growth inhibitory agents may be performed e.g. using a variety of bifunctional protein coupling agents including but not limited to N-succinimidyl pyridyldithiobutyrate (SPDB), butanoic acid 4-[(5-nitro-2-pyridinyl) dithio]-2,5-dioxo-1-pyrrolidinyl ester (nitro-SPDB), 4-(Pyridin-2-yldisulfanyl)-2-sulfo-butyric acid (sulfo-SPDB), N-succinimidyl (2-pyridyldithio) propionate (SPDP), succinimidyl (N-maleimidomethyl) cyclohexane-1-carboxylate (SMCC), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis (p-azidobenzoyl)-hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al (1987). Carbon labeled 1-isothiocyanatobenzyl methyldiethylene triaminepentaacetic acid (MX-DTPA) is an exemplary chelating agent for conjugation of radionucleotide to an antibody (WO 94/11026).

In embodiments of the present invention, the linker may be a “cleavable linker”, which may facilitate release of the cytotoxic drug or other growth inhibitory agent inside of or in the vicinity of a cell, e.g. a tumor cell. In some embodiments, the linker is a linker cleavable in an endosome of a mammalian cell. For example, an acid-labile linker, a peptidase-sensitive linker, an esterase labile linker, a photolabile linker or a disulfide-containing linker (see e.g. U.S. Pat. No. 5,208,020) may be used.

When referring to a structural formula representing an immunoconjugate, the following nomenclature is also used herein: a growth inhibitory agent and a linker, taken together, are also referred to as a [(linker)-(growth inhibitory agent)] moiety; for instance, an exatecan molecule and a linker, taken together, are also referred to as a [(linker)-(exatecan)] moiety.

In some specific embodiments of the present invention, the linker is a linker cleavable by the human enzyme glucuronidase. For example, an immunoconjugate prepared by the method of the present invention may thus have the following formula (IIA), which includes a linker cleavable by glucuronidase:

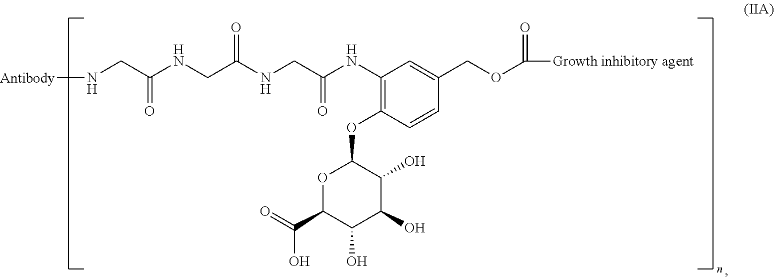

wherein the antibody is the antibody of the invention (preferably a monoclonal antibody), and wherein n is a number of [(linker)-(growth inhibitory agent)] moieties covalently linked to the antibody. In embodiments using formula IIA above, n is preferably between 3 and 4 and most preferably between 3.5 and 4.0 (i.e. about 4).

The number n is also referred to as “drug-to-antibody ratio” (or “DAR”); this number n is always to be understood as an average number for any given (preparation of an) immunoconjugate.

In the above formula (IIA), the chemical structure between the antibody and the growth inhibitory agent is a linker. One of these linkers is also contained in (VIIIA) depicted further below.

In any one of the embodiments with linkers cleavable by glucuronidase, as described above, the growth inhibitory agent may be exatecan, for example.

Accordingly, in some embodiments, the method of the present invention provides an immunoconjugate comprising an antibody according to the invention covalently linked via a linker to exatecan, wherein the conjugate has the following formula (IVA):

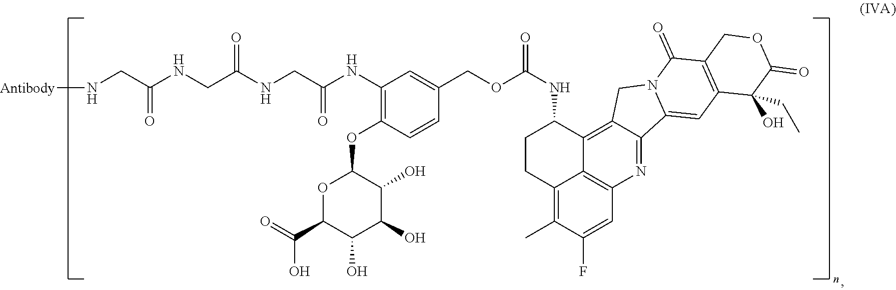

wherein n is a number of [(linker)-(exatecan)] moieties covalently linked to the antibody. The number n (also referred to as the DAR) may be e.g. between 1 and 10;. In embodiments using formula IVA above, n is preferably between 3 and 4 and most preferably between 3.5 and 4.0 (i.e. about 4).

Accordingly, in some embodiments, the method of the present invention provides an immunoconjugate comprising an antibody according to the invention covalently linked via a linker to exatecan, wherein the conjugate has the following formula (VIA):

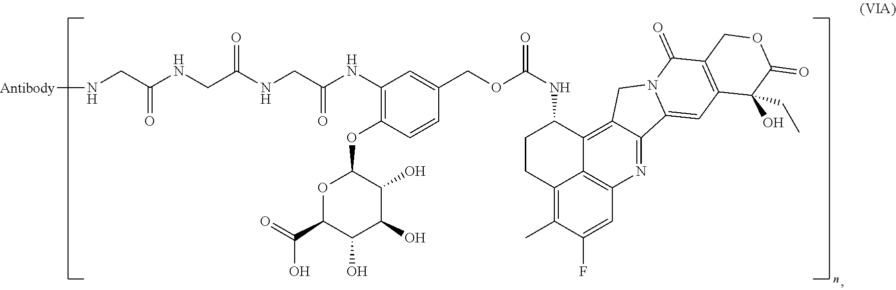

wherein n is a number of [(linker)-(exatecan)] moieties covalently linked to the antibody. The number n (also referred to as the DAR) may be e.g. between 1 and 10; In embodiments using formula VIA above, n is preferably between 3 and 5 and more preferably between 3.5 and 4.5 and most preferably 4.

In preparing any of the immunoconjugates described above using the method of the present invention, an antibody against any target may be used.

Accordingly, in some embodiments, the method of the present invention provides an immunoconjugate comprising an antibody according to the invention covalently linked via a linker to exatecan, wherein the conjugate has the following formula (VIIIA):

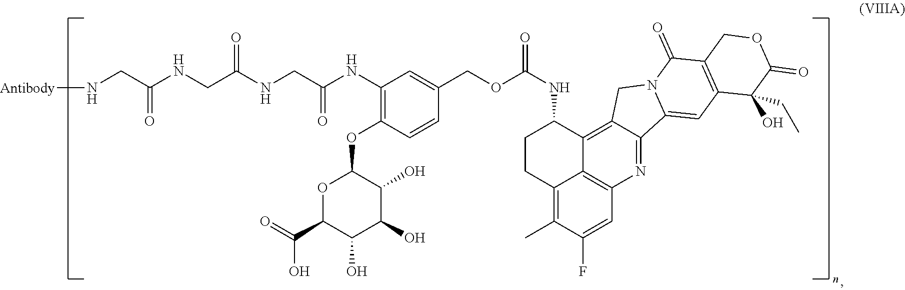

wherein n is a number of [(linker)-(exatecan)] moieties covalently linked to the antibody. The number n (also referred to as the DAR) may be e.g. between 1 and 10.

In other embodiments of the present invention, the linker may be a “non-cleavable linker” (for example an SMCC linker). Release of the growth inhibitory agent from the antibody can occur upon lysosomal degradation of the antibody.

In other embodiments of the invention, the immunoconjugate may be a fusion protein comprising an antibody of the invention and a cytotoxic or growth inhibitory polypeptide (as the growth inhibitory agent); such fusion proteins may be made by recombinant techniques or by peptide synthesis, i.e, methods well known in the art. A molecule of encoding DNA may comprise respective regions encoding the two portions of the conjugate (antibody and cytotoxic or growth inhibitory polypeptide, respectively) either adjacent to one another or separated by a region encoding a linker peptide.

The antibodies of the present invention may also be used in directed enzyme prodrug therapy such as antibody-directed enzyme prodrug therapy by conjugating the antibodies to a prodrug-activating enzyme which converts a prodrug (e.g. a peptidyl chemotherapeutic agent, see WO81/01145) to an active cytotoxic drug (see, for example, WO 88/07378 and U.S. Pat. No. 4,975,278). The enzyme component of an immunoconjugate useful for ADEPT may include any enzyme capable of acting on a prodrug in such a way as to convert it into its more active, cytotoxic form. Enzymes that are useful in this context include, but are not limited to, alkaline phosphatase useful for converting phosphate-containing prodrugs into free drugs; arylsulfatase useful for converting sulfate-containing prodrugs into free drugs; cytosine deaminase useful for converting non-toxic fluorocytosine into the anticancer drug 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. The enzymes can be covalently bound to the antibodies by techniques well known in the art, such as the use of the linkers discussed above.

Suitable methods for preparing an immunoconjugate are well known in the art (see e.g. Hermanson G. T., Bioconjugate Techniques, Third Edition, 2013, Academic Press). For instance, methods of conjugating a cytotoxic drug to an antibody via a linker that attaches covalently to cysteine residues of interchain disulfide bridges of the antibody are well known. In general, an immunoconjugate can be obtained e.g. by a process comprising the steps of:
(i) preparing a compound comprising the linker and the growth inhibitory agent (e.g. cytotoxic drug), also referred to herein as a “drug-linker compound”;
(ii) bringing into contact an optionally buffered aqueous solution of an antibody with a solution of the drug-linker compound;
(iii) then optionally separating the conjugate which was formed in (ii) from the unreacted antibody and/or drug-linker compound.

The aqueous solution of antibody can be buffered with buffers such as e.g. histidine, potassium phosphate, acetate, citrate or N-2-Hydroxyethylpiperazine-N′-2-ethanesulfonic acid (Hepes buffer). The buffer may be chosen depending upon the nature of the antibody. The drug-linker compound can be dissolved e.g. in an organic polar solvent such as dimethyl sulfoxide (DMSO) or dimethylacetamide (DMA).

For conjugation to the cysteine residues of an antibody, the antibody is subjected to reduction (e.g. using TCEP) before step (ii). Suitable reduction conditions to reduce only the interchain disulfide bonds are known in the art.

The reaction temperature for conjugation is usually between 2° and 40° C. The reaction time can vary and is typically from 1 to 24 hours. The reaction between the antibody and the drug-linker compound can be monitored by size exclusion chromatography (SEC) with a refractometric and/or UV detector. If the conjugate yield is too low, the reaction time can be extended.

A number of different chromatography methods can be used by the person skilled in the art in order to perform the separation of step (iii): the conjugate can be purified e.g. by SEC, adsorption chromatography (such as ion exchange chromatography, IEC), hydrophobic interaction chromatography (HIC), affinity chromatography, mixed-support chromatography such as hydroxyapatite chromatography, or high performance liquid chromatography (HPLC) such as reverse-phase HPLC. Purification by dialysis or filtration or diafiltration can also be used.

After step (ii) and/or (iii), the conjugate-containing solution can be subjected to an additional step (iv) of purification e.g. by chromatography, ultrafiltration and/or diafiltration. Such an additional step of purification e.g. by chromatography, ultrafiltration and/or diafiltration can also be performed with the antibody-containing solution after the reduction reaction, in cases where reduction is performed prior to conjugation.

The conjugate is recovered at the end of such a process in an aqueous solution. The drug-to-antibody ratio (DAR) is a number that can vary with the nature of the antibody and of the drug-linker compound used along with the experimental conditions used for the conjugation (such as the ratio (drug-linker compound)/(antibody), the reaction time, the nature of the solvent and of the cosolvent if any). Thus, the contact between the antibody and the drug-linker compound can lead to a mixture comprising several conjugates differing from one another by different drug-to-antibody ratios. The DAR that is determined is thus an average value.

An exemplary method which can be used to determine the DAR consists of measuring spectrophotometrically the ratio of the absorbance at of a solution of purified conjugate at λ_{D} and 280 nm. 280 nm is a wavelength generally used for measuring protein concentration, such as antibody concentration. The wavelength λ_{D} is selected so as to allow discriminating the drug from the antibody, i.e. as readily known to the skilled person, λ_{D} is a wavelength at which the drug has a high absorbance and λ_{D} is sufficiently remote from 280 nm to avoid substantial overlap in the absorbance peaks of the drug and antibody. For instance, λ_{D} may be selected as being 370 nm for exatecan (or for camptothecin or other camptothecin analogs), or 252 nm for maytansinoid molecules.

A method of DAR calculation may be derived e.g. from Antony S. Dimitrov (ed), LLC, 2009, Therapeutic Antibodies and Protocols, vol 525, 445, Springer Science: The absorbances for the conjugate at λ_{D} (A_{λD}) and at 280 nm (A₂₈₀) are measured either on the monomeric peak of the size exclusion chromatography (SEC) analysis (allowing to calculate the “DAR (SEC)” parameter) or using a classic spectrophotometer apparatus (allowing to calculate the “DAR (UV)” parameter). The absorbances can be expressed as follows:

$${A}_{λ D} = ( {C}_{D} \times {ε}_{D λ D} ) + ( {C}_{A} \times {ε}_{A λ D} )$$

$${A}_{280} = ( {C}_{D} \times {ε}_{D 280} ) + ( {C}_{A} \times {ε}_{A 280} )$$

wherein:
C_{D} and C_{A} are respectively the concentrations in the solution of the drug and of the antibody.
ε_{DAD} and ε_{D280} are respectively the molar extinction coefficients of the drug at λ_{D} and 280 nm.
ε_{AND} and ε_{A280} are respectively the molar extinction coefficients of the antibody at λ_{D} and 280 nm.

Resolution of these two equations with two unknowns leads to the following equations:

$${C}_{D} = [ ( {ε}_{A 280} \times {A}_{λ D} ) = ( {ε}_{A λ D} \times {A}_{280} ) ] / [ ( {ε}_{D λ D} \times {ε}_{A 280} ) - ( {ε}_{A λ D} \times {ε}_{D 280} ) ]$$

$${C}_{A} = [ {A}_{280 -} ( {C}_{D} \times {ε}_{D 280} ) ] / {ε}_{A 280}$$

The average DAR is then calculated from the ratio of the drug concentration to that of the antibody:DAR=C_{D}/C_{A}.

An alternative method for preparing an immunoconjugate is described in the following, and this is a method of the present invention. This method can in particular be used for antibodies that comprise comprises an amino acid sequence selected from the group consisting of GGTLQSPP, TLQSG, TLQSPP and TLQSA in at least one of its light chain constant regions (CL) and/or in at least one of its heavy chain constant regions (CH).Thus, a further aspect of the invention relates to a method for producing an antibody-linker-conjugate comprising the steps:
(1) providing an antibody that comprises an amino acid sequence selected from the group consisting of GGTLQSPP, TLQSG, TLQSPP and TLQSA and more preferably the amino acid sequence TLQSPP or GGTLQSPP (most preferably the sequence GGTLQSPP) in at least one and preferably both of its light chain constant regions (CL) and/or in at least one and preferably both of its heavy chain constant regions (CH);
(2) mixing together in a reaction buffer at least the following components:
   (a) said antibody provided in step (1);
   (b) a microbial transglutaminase preferably a transglutaminase comprising the amino acid sequence

(SEQ ID NO: 44)
MGSGSGSGTGEEKRSYAETHRLTADDVDDINALNESAPAASSAGPSFRA
PDSDERVTPPAEPLDRMPDPYRPSYGRAETIVNNYIRKWQQVYSHRDGR
KQQMTEEQREWLSYGCVGVTWVNSGQYPTNRLAFAFFDEDKYKNELKNG
RPRSGETRAEFEGRVAKDSFDEAKGFQRARDVASVMNKALENAHDEGAY
LDNLKKELANGNDALRNEDARSPFYSALRNTPSFKDRNGGNHDPSKMKA
VIYSKHFWSGQDRSGSSDKRKYGDPEAFRPDRGTGLVDMSRDRNIPRSP
TSPGESFVNFDYGWFGAQTEADADKTVWTHGNHYHAPNGSLGAMHVYES
KFRNWSDGYSDFDRGAYVVTFVPKSWNTAPDKVTQGWP;
or
(SEQ ID NO: 73)
FRAPDSDERVTPPAEPLDRMPDPYRPSYGRAETIVNNYIRKWQQVYSHR
DGRKQQMTEEQREWLSYGCVGVTWVNSGQYPTNRLAFAFFDEDKYKNEL
KNGRPRSGETRAEFEGRVAKDSFDEAKGFQRARDVASVMNKALENAHDE
GAYLDNLKKELANGNDALRNEDARSPFYSALRNTPSFKDRNGGNHDPSK
MKAVIYSKHFWSGQDRSGSSDKRKYGDPEAFRPDRGTGLVDMSRDRNIP
RSPTSPGESFVNFDYGWFGAQTEADADKTVWTHGNHYHAPNGSLGAMHV
YESKFRNWSDGYSDFDRGAYVVTFVPKSWNTAPDKVTQGWPLE;
or
(SEQ ID NO: 45)
FRAPDSDERVTPPAEPLDRMPDPYRPSYGRAETIVNNYIRKWQQVYSHR
DGRKQQMTEEQREWLSYGCVGVTWVNSGQYPTNRLAFAFFDEDKYKNEL
KNGRPRSGETRAEFEGRVAKDSFDEAKGFQRARDVASVMNKALENAHDE
GAYLDNLKKELANGNDALRNEDARSPFYSALRNTPSFKDRNGGNHDPSK
MKAVIYSKHFWSGQDRSGSSDKRKYGDPEAFRPDRGTGLVDMSRDRNIP
RSPTSPGESFVNFDYGWFGAQTEADADKTVWTHGNHYHAPNGSLGAMHV
YESKFRNWSDGYSDFDRGAYVVTFVPKSWNTAPDKVTQGWP;
or
(SEQ ID NO: 48)
DSDERVTPPAEPLDRMPDPYRPSYGRAETIVNNYIRKWQQVYSHRDGRK
QQMTEEQREWLSYGCVGVTWVNSGQYPTNRLAFAFFDEDKYKNELKNGR
PRSGETRAEFEGRVAKDSFDEAKGFQRARDVASVMNKALENAHDEGAYL
DNLKKELANGNDALRNEDARSPFYSALRNTPSFKDRNGGNHDPSKMKAV
IYSKHFWSGQDRSGSSDKRKYGDPEAFRPDRGTGLVDMSRDRNIPRSPT
SPGESFVNFDYGWFGAQTEADADKTVWTHGNHYHAPNGSLGAMHVYESK
FRNWSDGYSDFDRGAYVVTFVPKSWNTAPDKVTQGWPLE;
or
(SEQ ID NO: 49)
DSDERVTPPAEPLDRMPDPYRPSYGRAETIVNNYIRKWQQVYSHRDGRK
QQMTEEQREWLSYGCVGVTWVNSGQYPTNRLAFAFFDEDKYKNELKNGR
PRSGETRAEFEGRVAKDSFDEAKGFQRARDVASVMNKALENAHDEGAYL
DNLKKELANGNDALRNEDARSPFYSALRNTPSFKDRNGGNHDPSKMKAV
IYSKHFWSGQDRSGSSDKRKYGDPEAFRPDRGTGLVDMSRDRNIPRSPT
SPGESFVNFDYGWFGAQTEADADKTVWTHGNHYHAPNGSLGAMHVYESK
FRNWSDGYSDFDRGAYVVTFVPKSWNTAPDKVTQGWP;
and

(c) a linker that comprises an H2N-moiety capable of reacting with the antibody from step (1) in the presence of said transglutaminase and wherein the linker is preferably a drug-linker where said linker is covalently attached to a drug;
(3) separating the antibody-linker-conjugate produced in step (2) from unreacted linker and from said transglutaminase preferably by subjecting said mixture from step (2) to a size-exclusion chromatography.

In one embodiment, the transglutaminase is encoded by the polynucleotide

(SEQ ID NO: 46)
ATGGGTAGCGGTAGCGGTTCAGGCACCGGTGAAGAAAAACGTAGCTATG
CAGAAACCCATCGTCTGACCGCAGATGATGTTGATGATATTAATGCACT
GAATGAAAGCGCACCGGCAGCAAGCAGCGCAGGTCCGAGCTTTCGTGCA
CCGGATAGTGATGAACGTGTTACCCCTCCGGCAGAACCGCTGGATCGTA
TGCCGGATCCGTATCGTCCGAGCTATGGTCGTGCCGAAACCATTGTTAA
TAACTATATTCGTAAGTGGCAGCAGGTTTATAGCCATCGTGATGGTCGT
AAACAGCAGATGACCGAAGAACAGCGTGAATGGCTGAGTTATGGTTGTG
TTGGTGTTACCTGGGTTAATAGCGGTCAGTATCCGACCAATCGTCTGGC
ATTTGCATTTTTCGATGAGGACAAATACAAGAACGAGCTGAAAAATGGT
CGTCCGCGTAGCGGTGAAACCCGTGCAGAATTTGAAGGTCGTGTTGCAA
AAGATAGCTTTGATGAAGCAAAAGGTTTTCAGCGTGCACGTGATGTTGC
AAGCGTTATGAATAAAGCACTGGAAAATGCACATGATGAAGGTGCCTAT
CTGGACAACCTGAAAAAAGAACTGGCAAATGGTAATGATGCCCTGCGTA
ATGAAGATGCACGTAGCCCGTTTTATAGCGCACTGCGTAATACCCCGAG
CTTTAAAGATCGTAATGGTGGTAATCATGACCCGAGCAAAATGAAAGCA
GTGATCTATAGCAAACACTTTTGGAGCGGTCAGGATCGTAGTGGTAGCA
GCGATAAACGTAAATATGGTGATCCGGAAGCATTTCGTCCGGATCGTGG
CACCGGTCTGGTTGATATGAGCCGTGATCGTAATATTCCGCGTAGTCCG
ACCAGTCCGGGTGAAAGCTTTGTTAATTTTGATTATGGTTGGTTTGGCG
CACAGACCGAAGCAGATGCAGATAAAACCGTTTGGACCCATGGCAATCA
TTATCATGCACCGAATGGTAGCCTGGGTGCAATGCATGTTTATGAAAGC
AAATTTCGCAATTGGAGCGACGGCTATAGCGATTTTGATCGTGGTGCCT
ATGTTGTTACCTTTGTTCCGAAAAGCTGGAATACCGCTCCGGATAAAGT
TACCCAAGGTTGGCCGTAA.

In a preferred embodiment of the method, said reaction buffer is 7% DMSO, 24 mM HEPES, pH 7.0.

In the method of the invention, said linker is a linker having the formula

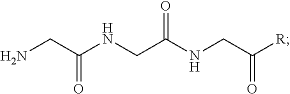

wherein R is the remainder of the linker and may optionally also comprise a drug, whereby the drug is preferably exatecan. In more preferred embodiments of the method of the invention the linker is NH2-GGG-beta-glucuronide.

In a exemplary embodiment of the method of the invention in step (2) the mixture comprises the following drug-linker:

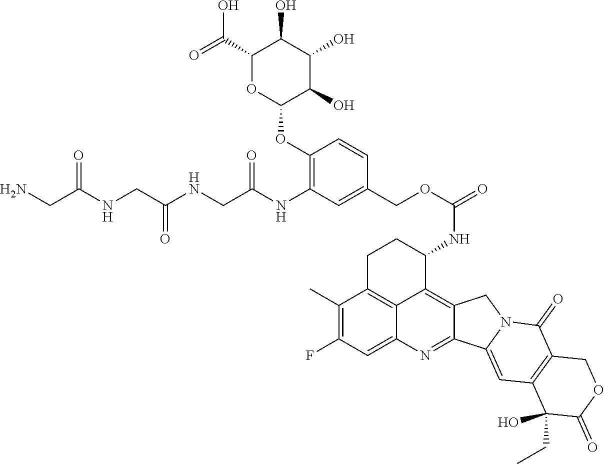 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CN)c1)CC3]

In a further embodiment of the method of the invention, the mixture in step (2) comprises 5 molar equivalents of linker or drug-linker, respectively, per conjugation site, wherein a conjugation site is a sequence GGTLQSPP, TLQSG, TLQSPP or TLQSA comprised in the light chain constant regions (CL) and/or in the heavy chain constant region (CH) of said antibody.

A further aspect of the invention relates to an antibody-linker conjugate producible according to the method of the invention.

Exemplary methods of the invention for preparing an immunoconjugate are described in the Examples.

### Drug-Linker Compounds

The present disclosure also provides compounds comprising a linker and a growth inhibitory agent (e.g. a cytotoxic drug), also referred to herein as “drug-linker compounds”. For instance, the present disclosure provides a compound of the following formula (XA):

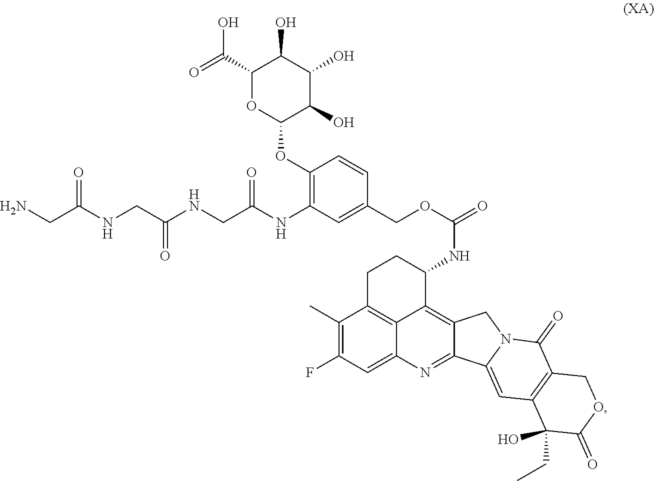 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CN)c1)CC3]

or a physiologically acceptable salt thereof; the compound according to formula XA is also referred to herein as “drug-linker compound 1-M”, “compound DL1-M” or “DL1-M”.

These drug-linker compounds may be used to prepare immunoconjugates using the method of the invention as described herein above and below.

The exemplary drug-linker compounds disclosed (e.g. those of (XA) depicted above) may be prepared by chemical synthesis, for instance as described in the Examples further below.

### Pharmaceutical Compositions

The antibodies or immunoconjugates of the invention may be combined with pharmaceutically acceptable carriers, diluents and/or excipients, and optionally with sustained-release matrices including but not limited to the classes of biodegradable polymers, non-biodegradable polymers, lipids or sugars, to form pharmaceutical compositions.

Thus, another aspect of the invention relates to a pharmaceutical composition comprising an antibody or an immunoconjugate of the invention and a pharmaceutically acceptable carrier, diluent and/or excipient.

### Therapeutic Methods and Uses

The present invention also provides an immunoconjugate (obtainable by a method of the invention) or pharmaceutical composition of the invention comprising such immunoconjugate for use as a medicament. For instance, the invention provides the immunoconjugate or pharmaceutical composition of the invention for use in the treatment of cancer. The invention further provides a method of treating cancer, comprising administering the immunoconjugate or pharmaceutical composition of the invention to a subject in need thereof.

The antibodies disclosed herein and any other antibodies may be conjugated (linked) to a growth inhibitory agent or to another agent using the method of the invention. The antibodies will thereby be useful for targeting said drug (e.g. growth inhibitory agent) to the target cells (e.g. cancerous cells) expressing or over-expressing the antigen (preferably a cell-surface antigen) that the antibodies bind to.

In some embodiments, an antibody that can be used in the method of the invention may be an antibody with a modified amino acid sequence that results in reduced or eliminated binding to most Fcγ receptors, which can reduce uptake and toxicity in normal cells and tissues expressing such receptors, e.g. macrophages, liver sinusoidal cells etc.,

An aspect of the invention relates to a method of treating cancer, comprising administering a therapeutically effective amount of the immunoconjugate or pharmaceutical composition of the invention to a subject in need thereof.

Efficacy of the treatment with an antibody or immunoconjugate or pharmaceutical composition according to the invention may be readily assayed in vivo, for instance in a mouse model of cancer and by measuring e.g. changes in tumor volume between treated and control groups, % tumor regression, partial regression or complete regression.

### Kits

The invention provides in a further aspect a kit of parts comprising a reaction buffer as defined herein in the context of the method of the invention and wherein the kit comprises a microbial transglutaminase from S. ladakanum preferably a transglutaminase comprising the amino acid sequence as defined in SEQ ID NO:s 44, 47, 48, 49 or 45 and preferably comprising the amino acid sequence of SEQ ID NO 45.

In one embodiment of the kit of parts of the invention, the kit further comprises a linker having a primary amino group wherein the linker is a substrate of the microbial transglutaminase and wherein the linker is preferably further covalently attached to a payload. Preferred linkers having the formula:

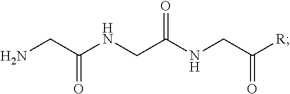

wherein R is the remainder of the linker and may optionally also comprise a drug or detectable label. Examples for a detectable label include a radioactive group and a fluorescent group.

In one embodiment of the kit of parts of the invention, the kit further comprises an instruction manual with instructions how to use the reaction buffer and the microbial transglutaminase of the kit to conjugate a linker with a primary amino group to an antibody, preferably a monoclonal antibody.

### Conjugation Methods of the Invention

As shown in the examples, the inventors have developed surprisingly improved methods to effectively covalently conjugate an antibody to a primary amine of a linker using a system based on a transglutaminase enzyme.

Accordingly, the invention provides in a further aspect a method for producing an antibody-linker-conjugate comprising the steps:
(1) providing an antibody that optionally comprises an amino acid sequence selected from the group consisting of GGTLQSPP, TLQSG, TLQSPP, GGTLQSG and TLQSA and more preferably the amino acid sequence TLQSPP or GGTLQSPP (most preferably the amino acid sequence GGTLQSPP) in at least one and preferably both of its light chain constant regions (CL) and/or in at least one and preferably both of its heavy chain constant regions (CH);
(2) mixing together in a reaction buffer at least the following components:
   (a) said antibody provided in step (1);
(b) a microbial transglutaminase, preferably a transglutaminase comprising the amino acid sequence as defined in SEQ ID NO:s 44, 47, 48, 49, 45, 73, 81, 82, 83 or 84 and preferably comprising the amino acid sequence of SEQ ID NO 45; and
(c) a linker or therapeutic agent that comprises an H2N-moiety capable of reacting with the antibody from step (1) in the presence of said transglutaminase and wherein the linker is preferably a drug-linker where said linker is covalently attached to a drug;
(3) separating the antibody-linker-conjugate produced in step (2) from unreacted linker and from said transglutaminase preferably by subjecting said mixture from step (2) to a size-exclusion chromatography.

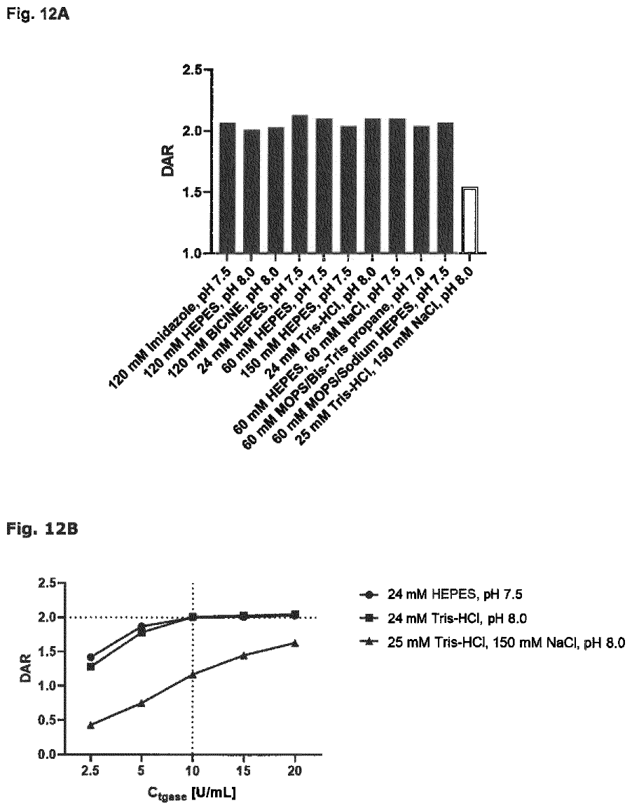

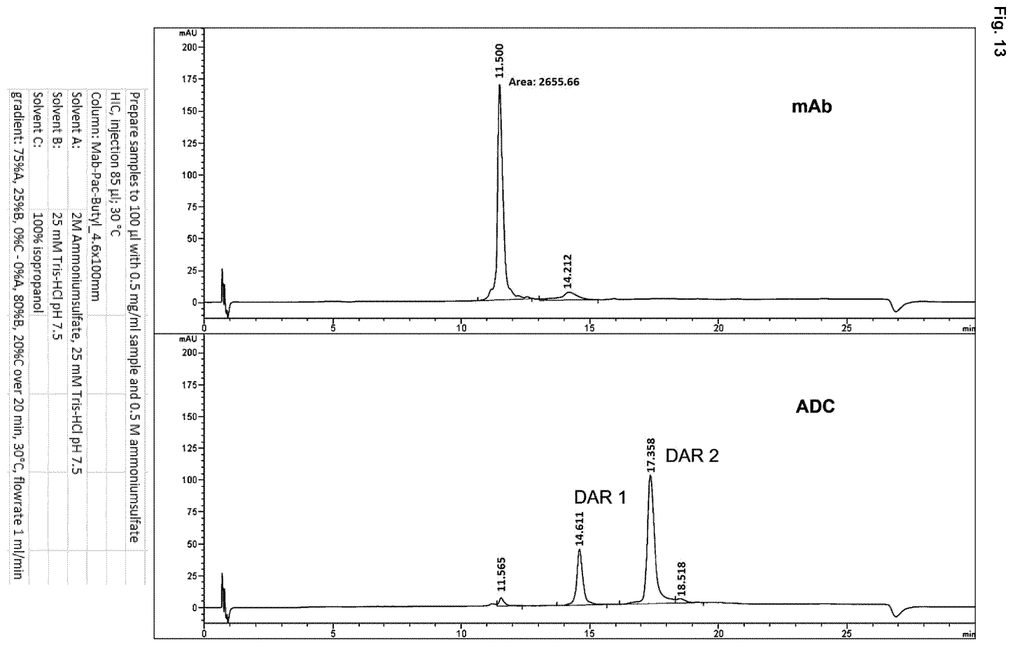

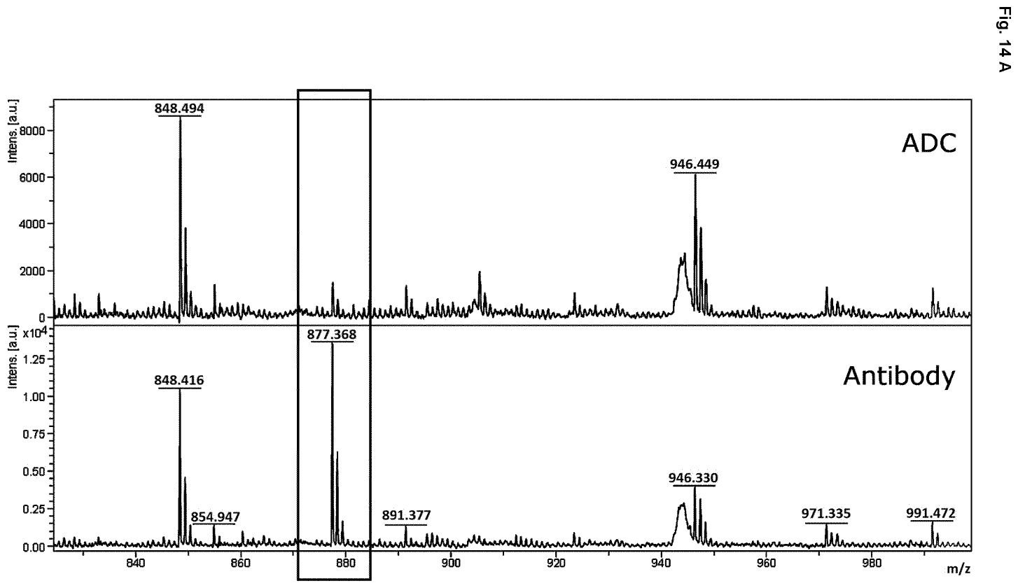

In method step (1) the antibody optionally comprises an amino acid sequence selected from the group consisting of GGTLQSPP, TLQSG, TLQSPP, GGTLQSG and TLQSA. Such a sequence is useful because it comprises a glutamine that can react with said linker or said therapeutic agent in method step (2) (c). However, the conjugation reaction can also be carried out using a native antibody as shown, for example, in FIG. 12. Thus, if an antibody does not comprise such amino acid sequence as shown above, it is preferred that an antibody is used in the method of the invention that comprises a glutamine at position 295 (Eu numbering) or a glutamine at a different position that is capable of reacting with said linker or said therapeutic agent in method step 2 (c) in the presence of said reaction buffer and said microbial transglutaminase.

In method step (2) (c) the H2N-moiety of the linker or of the therapeutic agent reacts with a glutamine of said antibody. The glutamine of the antibody can, for example, be glutamine at position 295 (Eu numbering) of the heavy chain of the antibody and/or can be glutamine in an amino acid sequence of said antibody, wherein said amino acid sequence is selected from the group consisting of GGTLQSPP, TLQSG, TLQSPP, GGTLQSG and TLQSA.

Preferably, said reaction buffer is a reaction buffer that is between 0% and 7% DMSO, between 20 and 30 mM HEPES (preferably 24 mM HEPES) and pH of between 6.8 and 7.4 (preferably pH 7); or is a reaction buffer that comprises between 3% and 10% DMSO, between 5 and 100 mM HEPES, and comprises less than 150 mM NaCl and has a pH of between 6 and 8; or is a reaction buffer that comprises between 3% and 10% DMSO, between 5 and 60 mM HEPES, and comprises less than 10 mM NaCl (preferably no NaCl) and has a pH of between 6 and 7.5.

In a further embodiment of the method of the invention said linker is a linker having the formula:

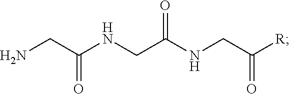

wherein R is the remainder of the linker which may also include a therapeutic agent or detectable label.

In a further embodiment of the method of the invention in step (2) of the method the mixture comprises the following drug-linker:

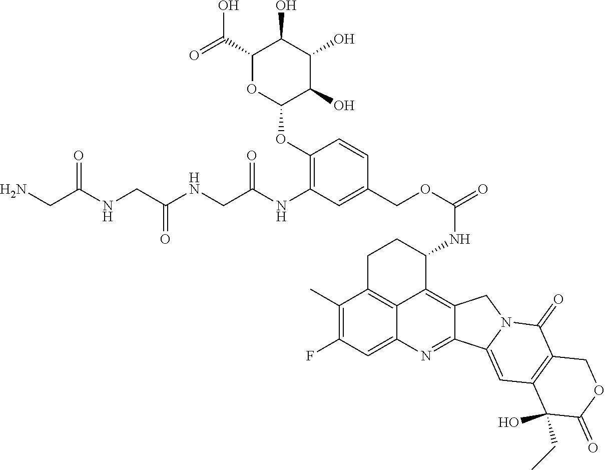 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CN)c1)CC3]

In a further embodiment of the method of the invention the mixture in step (2) comprises 5 molar equivalents of linker or drug-linker, respectively, per conjugation site, wherein a conjugation site is a sequence GGTLQSPP, TLQSG, TLQSPP, GGTLQSG or TLQSA comprised in the light chain constant regions (CL) and/or in the heavy chain constant region (CH) of said antibody; and/or wherein a conjugation site is a glutamine at position 295 of the heavy chain of the antibody, wherein the antibody is a monoclonal antibody and wherein Eu numbering is used for defining the position of said glutamine at position 295.

In a further embodiment of the method of the invention the antibody is a monoclonal antibody and preferably an anti-CEACAM5 antibody as defined in any of (a), (b) or (c) as specified in the following:
(a) the antibody comprises
   (i) at least one light chain constant region (CL) that comprises a sequence selected from the group consisting of GGTLQSPP, TLQSG, TLQSPP, GGTLQSG and TLQSA, and preferably comprising this sequence at the C-terminus of said light chain constant region; and
   (ii) at least one heavy chain constant region (CH) comprising one or more of the following amino acid substitutions:
      (a) L234A and L235A (LALA mutation);
      (b) L234A and L235A and P329G (LALA-PG mutation);
      (c) L235A and G237A (LAGA mutation);
      (d) M252Y and S254T and T256E (YTE mutation);
      (e) K222R;
   and wherein Eu numbering is used for said amino acid substitutions;
   and wherein said isolated antibody comprises a CDR1-H consisting of the amino acid sequence of SEQ ID NO: 3, a CDR2-H consisting of the amino acid sequence of SEQ ID NO: 4, a CDR3-H consisting of the amino acid sequence of SEQ ID NO: 5, a CDR1-L consisting of the amino acid sequence of SEQ ID NO: 6, a CDR2-L consisting of the amino acid sequence of SEQ ID NO: 7, and a CDR3-L consisting of the amino acid sequence of SEQ ID NO: 8;
(b) wherein said heavy chain constant regions (CH) and light chain constant regions (CL) of the antibody have any of the following sequence combinations:
   (1) both CH comprise a sequence of SEQ ID NO: 31 and both CL comprise a sequence of SEQ ID NO: 12; or
   (2) both CH comprise a sequence of SEQ ID NO: 31 and both CL comprise a sequence of SEQ ID NO: 33; or
   (3) both CH comprise a sequence of SEQ ID NO: 32 and both CL comprise a sequence of SEQ ID NO: 33; or
   (4) both CH comprise a sequence of SEQ ID NO: 50 and both CL comprise a sequence of SEQ ID NO: 12; or
   (5) both CH comprise a sequence of SEQ ID NO: 50 and both CL comprise a sequence of SEQ ID NO: 33; or
   (6) one CH comprise a sequence of SEQ ID NO: 31 and one CL comprise a sequence of SEQ ID NO: 12; or
   (7) one CH comprise a sequence of SEQ ID NO: 31 and one CL comprise a sequence of SEQ ID NO: 33; or
   (8) one CH comprise a sequence of SEQ ID NO: 32 and one CL comprise a sequence of SEQ ID NO: 33; or
   (9) one CH comprise a sequence of SEQ ID NO: 50 and one CL comprise a sequence of SEQ ID NO: 12; or
   (10) one CH comprise a sequence of SEQ ID NO: 50 and one CL comprise a sequence of SEQ ID NO: 33.
(c) the antibody consists of
   two identical heavy chains (HC) comprising the amino acid sequence of SEQ ID NO: 34 and two identical light chains (LC) comprising the amino acid sequence of SEQ ID NO: 14; or
   two identical heavy chains (HC) comprising the amino acid sequence of SEQ ID NO: 34 and two identical light chains (LC) comprising the amino acid sequence of SEQ ID NO: 36; or
   two identical heavy chains (HC) comprising the amino acid sequence of SEQ ID NO: 35 and two identical light chains (LC) comprising the amino acid sequence of SEQ ID NO: 36; or
   two identical heavy chains (HC) comprising the amino acid sequence of SEQ ID NO: 51 and two identical light chains (LC) comprising the amino acid sequence of SEQ ID NO: 14; or
   two identical heavy chains (HC) comprising the amino acid sequence of SEQ ID NO: 51 and two identical light chains (LC) comprising the amino acid sequence of SEQ ID NO: 36.

and/or

wherein the drug is a growth inhibitory agent as defined in (d), (e) or (f) as specified in the following:
(d) the growth inhibitory agent is a cytotoxic drug or a radioactive moiety.
(e) the growth inhibitory agent is selected from a group consisting of chemotherapeutic agents, enzymes, antibiotics, toxins such as small molecule toxins or enzymatically active toxins, toxoids, vincas, taxanes, maytansinoids or maytansinoid analogs, tomaymycin or pyrrolobenzodiazepine derivatives, cryptophycin derivatives, leptomycin derivatives, auristatin or dolastatin analogs, prodrugs, topoisomerase I inhibitors, topoisomerase II inhibitors, DNA alkylating agents, anti-tubulin agents, CC-1065 and CC-1065 analogs;
(f) the growth inhibitory agent is exatecan.

The invention further provides an antibody-linker conjugate producible according to any of claims 1-7, wherein the linker is a linker or drug linker as defined in the following:

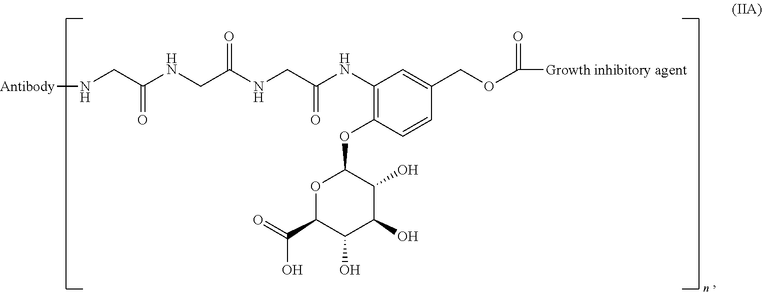

wherein n is a number of [(linker) (growth inhibitory agent)] moieties covalently linked to the antibody; or

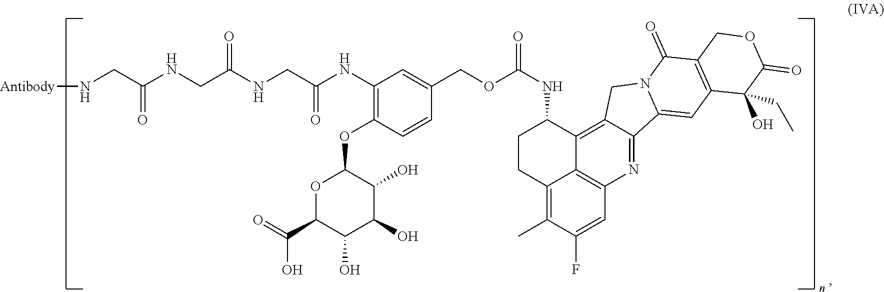

wherein n is a number of [(linker)-(exatecan)] moieties covalently linked to the antibody; and wherein n is preferably between 1 and 10 and more preferably 4.

In a further embodiment of the antibody-linker conjugate of the invention the linker is a drug linker and wherein the drug is a growth inhibitory agent and preferably exatecan.

In a further embodiment of the method of the invention in step (2) the mixture comprises the following drug-linker:

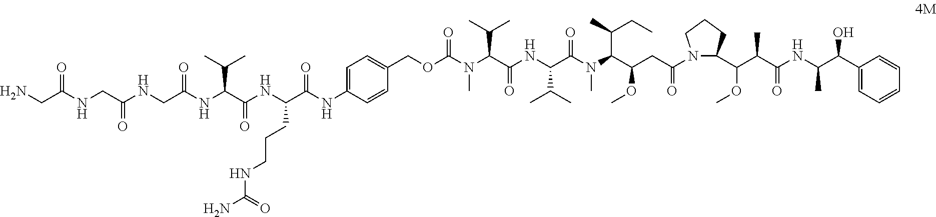 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CNC(=O)CNC(=O)CN)C(C)C)cc1)C(C)C]

In a further embodiment of the method of the invention the glutamine of the amino acid sequence selected from the group consisting of GGTLQSPP, TLQSG, TLQSPP, GGTLQSG and TLQSA of said antibody reacts in step (2) of the method in the presence of said microbial transglutaminase to form a covalent bond with said H2N-moiety of the linker.

In a further embodiment of the method of the invention the antibody is a monoclonal antibody.

In a further embodiment of the method of the invention the microbial transglutaminase is a microbial transglutaminase from S. ladakanum.

In a further embodiment of the method of the invention the reaction mixture obtained in step (2) is incubated at a temperature between 20° C. and 38° C. until the antibody-linker-conjugate has formed and can be separated in method step (3).

In a further embodiment of the method of the invention the reaction buffer comprises one or more of the following buffers adjusted to a pH of between 7 and pH 8.8: BICINE, BICINE/Tris, Tris-HCl, HEPES and/or Tricine.

In a further embodiment of the method of the invention wherein the reaction buffer comprises Tris-HCl adjusted to a pH of between 7.7 and 8.5: or comprises BICINE and/or BICINE/Tris adjusted to a pH of between 7.8 and 8.8: or comprises Tricine adjusted to a pH of between 7.8 and 8.0.

In a further embodiment of the method of the invention the reaction buffer comprises HEPES in an amount of between 5 mM and 100 mM.

In a further embodiment of the method of the invention the reaction buffer is a reaction buffer that comprises between 3% and 10% DMSO, between 5 and 60 mM HEPES, and comprises less than 100 mM NaCl (preferably no NaCl) and has a pH of between 6 and 7.5; said antibody further comprises a GGTLQSPP, TLQSG, TLQSPP, GGTLQSG and TLQSA sequence in both light chain constant regions (CL) and/or in both heavy chain constant regions (CH); and said linker is a linker having the formula:

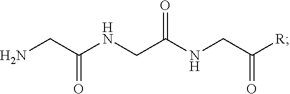

wherein R is the remainder of the linker and may optionally also comprise a drug.

In a further embodiment of the method of the invention said reaction buffer comprises less than 150 mM NaCl, preferably less than 100 mM NaCl, more preferably less than 75 mM NaCl, even more preferably 5 mM NaCl or less, most preferably wherein said reaction buffer comprises no NaCl.

In a further embodiment of the method of the invention the total amount of all buffering agents that are comprised in said reaction buffer is lower than 160 mM and preferably lower than 155 mM.

In a further embodiment of the method of the invention the total amount of all buffering agents that are comprised in said reaction buffer is lower than 70 mM.

In a further embodiment of the method of the invention the total amount of all buffering agents that are comprised in said reaction buffer is lower than 155 mM; and wherein the reaction buffer comprises less than 10 mM NaCl and wherein the total amount of microbial transglutaminase is between 2.5 and 12 U/ml.

In a further embodiment of the method of the invention the total amount of all buffering agents that are comprised in said reaction buffer is lower than 70 mM; and wherein the reaction buffer comprises less than 50 mM NaCl; wherein the total amount of microbial transglutaminase is between 2.5 and 12 U/ml and wherein the pH of the reaction buffer is between pH 5.9 and pH 8.1.

In a further embodiment of the method of the invention the antibody is trastuzumab, rituximab or labetuzumab; or wherein said antibody comprises the amino acid Q295 in its heavy chain constant regions (CH), wherein Eu numbering is used for defining the position of said glutamine at position 295; or. wherein said antibody comprises the amino acid Q295 in its heavy chain constant regions (CH), wherein Eu numbering is used for defining the position of said glutamine at position 295; and wherein said antibody is an IgG monoclonal antibody.

In a further embodiment of the method of the invention said reaction buffer comprises at least one buffering agent selected from the group consisting of Imidazole, HEPES, BICINE, Tris-HCl, MOPS/Bis-Tris propane and MOPS/Sodium HEPES, and wherein the buffering agent concentration is selected within the range of 10-160 mM, preferably wherein the buffering agent is HEPES at a concentration of between 10 and 30 mM, more preferably at a concentration of about 24 mM.

In a further embodiment of the method of the invention said reaction buffer has a pH of between 6.5 and 8.7, more preferably a pH of 6.8 to 8.2, most preferably a pH of about 7; or wherein said reaction buffer has a pH of between about 7 and 8.5 and wherein the reaction buffer comprises less than 10 mM NaCl and between 20 and 120 mM of at least one of the following buffering agents: HEPES, Tris-HCl, MOPS/NaHEPES, BICINE, BICINE/Tris, Imidazole and Tricine.

In a further embodiment of the method of the invention said reaction buffer comprises between 4% and 7% DMSO.

In a further embodiment of the method of the invention said reaction buffer comprises said transglutaminase wherein said transglutaminase comprises the amino acid sequence of SEQ ID NO: 45 and wherein said microbial transglutaminase is comprised in the mixture in step (2) of the method at a concentration of between 5 U/mL and 25 U/mL and preferably at a concentration of between 10 U/mL and 20 U/mL.

In a further embodiment of the method of the invention said reaction buffer comprises no sodium chloride and further comprises about 24 mM HEPES, wherein said reaction buffer has a pH of about 7.5 and wherein the microbial transglutaminase concentration is about 12.5 U/mL.

In a further embodiment of the method of the invention said reaction buffer comprises no sodium chloride and further comprises about 24 mM HEPES, wherein said reaction buffer has a pH of about 7 and comprises about 4% DMSO, wherein the microbial transglutaminase concentration in the mixture in step (2) of the method is about 20 U/mL.

In a further embodiment of the method of the invention said reaction buffer comprises no sodium chloride and further comprises about 24 mM HEPES, wherein said reaction buffer has a pH of about 7 and comprises about 7% DMSO, wherein the microbial transglutaminase concentration in the mixture of step (2) of the method is about 20 U/mL.

In a further embodiment of the method of the invention said antibody comprises the amino acid sequence TLQSG, GGTLQSPP or GGTLQSG at the C-terminus of its light chain constant regions (CL) and the amino acid Q295 in its heavy chain constant regions (CH), wherein Eu numbering is used for defining the position of said glutamine at position 295.

In a further embodiment of the method of the invention the drug antibody ratio (DAR) of the antibody-linker-conjugate produced in step (2) is between 3 and 4.5, preferably 3.8 to 4.1.

In a further embodiment of the method of the invention the drug antibody ratio (DAR) of the antibody-linker-conjugate produced in step (2) is between 1.9 and 2.

In a further embodiment of the method of the invention said antibody comprises the amino acid Q295 in its heavy chain constant regions (CH), wherein Eu numbering is used for defining the position of said glutamine at position 295; and wherein said antibody is an IgG monoclonal antibody.

In a further embodiment of the method of the invention the mTG enzyme is from S. ladakanum;

wherein said antibody is a monoclonal antibody comprising glutamine at position 295 and wherein the reaction buffer comprises DMSO and further comprises HEPES in an amount of between about 20 to 30 mM; wherein the reaction buffer comprises less than 100 mM NaCl and wherein the pH of the reaction buffer is between about pH 7 and pH 7.5; and wherein Eu numbering is used for defining the position of said glutamine at position 295.

In a further embodiment of the method of the invention the mTG enzyme is from S. ladakanum; wherein said antibody is a monoclonal antibody comprising glutamine at position 295 and wherein the reaction buffer comprises DMSO and further comprises HEPES in an amount of between about 20 to 30 mM; wherein the reaction buffer comprises less than 100 mM NaCl and wherein the pH of the reaction buffer is between about pH 7 and pH 7.5; and wherein Eu numbering is used for defining the position of said glutamine at position 295; and wherein the C-terminus of the light chain of the antibody comprises the amino acid sequence GGTLQSPP.

In a further embodiment of the method of the invention the linker has a structure (A) or (B) shown below, wherein R is a therapeutic agent or another payload:

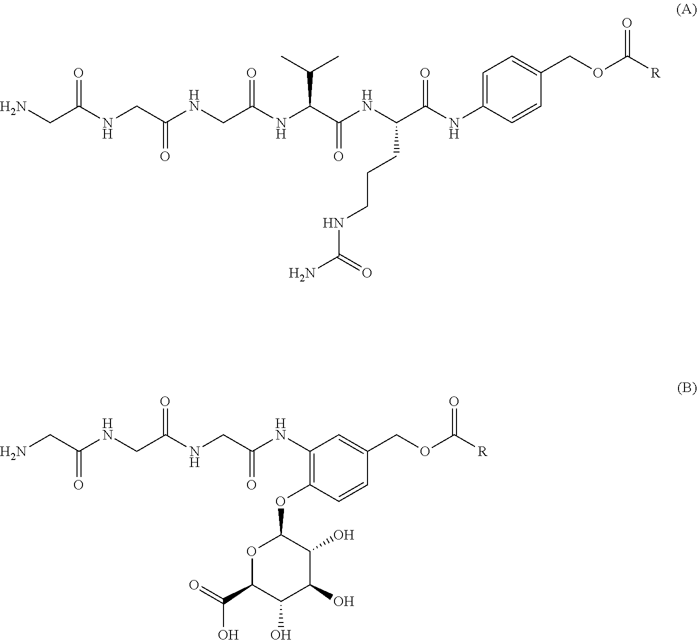

In a further embodiment of the method of the invention the antibody comprises one or more amino acid sequence selected from the group consisting of GGTLQSPP, TLQSG, TLQSPP, GGTLQSG and TLQSA; wherein this amino acid sequence is located at the C-terminus of the light chain of the antibody and/or at the C-terminus of the heavy chain of the antibody.

In a further embodiment of the method of the invention in step (2) of the method a molar excess of drug linker is used per conjugation size of the antibody and preferably at least 4 molar equivalents of drug-linker per conjugation site are used.

In a further embodiment of the antibody-drug-conjugate of the invention the immunoconjugate has formula (IVA) and n is between 1 and 10 and preferably about 4.

In a further aspect, the invention provides an antibody comprising an amino acid sequence selected from the group consisting of GGTLQSPP, GGTLQSG and TLQSA at the C-terminus of the heavy chain of the antibody and/or comprises an amino acid sequence selected from the group consisting of GGTLQSPP, GGTLQSG and TLQSA at the C-terminus of the light chain of the antibody.

### BRIEF DESCRIPTION OF THE SEQUENCES

Amino acid sequences:

| | |
|---|---|
| SEQ ID NO: 1 | Human CEACAM5 protein sequence according |
| | to GenBank accession number AAA51967.1 |
| SEQ ID NO: 2 | Macaca fascicularis CEACAM5 protein sequence |
| | (NCBI Reference Sequence XP_005589491.1) |
| SEQ ID NO: 3 | CDR1-H of mAb1 |
| SEQ ID NO: 4 | CDR2-H of mAb1 |
| SEQ ID NO: 5 | CDR3-H of mAb1 |
| SEQ ID NO: 6 | CDR1-L of mAb1 |
| SEQ ID NO: 7 | CDR2-L of mAb1 |
| SEQ ID NO: 8 | CDR3-L of mAb1 |
| SEQ ID NO: 9 | VH of mAb1 |
| SEQ ID NO: 10 | VL of mAb1 |
| SEQ ID NO: 11 | CH of mAb1 |
| SEQ ID NO: 12 | CL of mAb1, of mAb1-M and of mAb6-M |
| SEQ ID NO: 13 | HC of mAb1 |
| SEQ ID NO: 14 | LC of mAb1, of mAb1-M and of mAb6-M |

Nucleic acid sequences:

| | |
|---|---|
| SEQ ID NO: 15 | DNA sequence encoding HC of mAb1 |
| SEQ ID NO: 16 | DNA sequence encoding LC of mAb1 |

Amino acid sequences:

| | |
|---|---|
| SEQ ID NO: 17 | HC of antibody hu8G4 |
| SEQ ID NO: 18 | LC of antibody hu8G4 |
| SEQ ID NO: 19 | HC of optimized antibody Variant 1 |
| SEQ ID NO: 20 | LC of optimized antibody Variant 1 |
| SEQ ID NO: 21 | LC of optimized antibody Variant 2 |
| SEQ ID NO: 22 | LC of optimized antibody Variant 4 |
| SEQ ID NO: 23 | LC of optimized antibody Variant 5 |
| SEQ ID NO: 24 | HC of optimized antibody Variant 6 |
| SEQ ID NO: 25 | HC of huMab2-3 (allotype) |
| SEQ ID NO: 26 | LC of huMab2-3 |
| SEQ ID NO: 27 | HC of hmn-14 |
| SEQ ID NO: 28 | LC of hmn-14 |
| SEQ ID NO: 29 | HC of rb8G4 |
| SEQ ID NO: 30 | LC of rb8G4 |
| SEQ ID NO: 31 | CH of mAb1-M & mAb2-M (CH-LALA-YTE) |
| SEQ ID NO: 32 | CH of mAb3-M (CH-K222R-LALA-YTE) |
| SEQ ID NO: 33 | CL of mAb2-M, of mAb3-M and of |
| | mAb7-M (CL-Tag) |
| SEQ ID NO: 34 | HC of mAb1-M and of mAb2-M (HC-LALA-YTE) |
| SEQ ID NO: 35 | HC of mAb3-M (HC-K222R-LALA-YTE) |
| SEQ ID NO: 36 | LC of mAb2-M, of mAb3-M and of |
| | mAb7-M (LC-Tag) |
| SEQ ID NO: 40 | HC of mAb5-M (antiCD20-HC) |
| SEQ ID NO: 41 | LC of mAb5-M (antiCD20-LC) |
| SEQ ID NO: 44 | Transglutaminase |
| SEQ ID NO: 45 | Transglutaminase (activated, version1) |
| SEQ ID NO: 47 | Transglutaminase (activated, from S. mobaraensis) |
| SEQ ID NO: 48 | Transglutaminase (activated, version3) |
| SEQ ID NO: 49 | Transglutaminase (activated, version4) |
| SEQ ID NO: 50 | CH of mAb6-M and of mAb7-M (CH-LALA) |
| SEQ ID NO: 51 | HC of mAb6-M and of mAb7-M (HC-LALA) |
| SEQ ID NO: 54 | Framework region FR1 |
| SEQ ID NO: 55 | Framework region FR2 |
| SEQ ID NO: 56 | Framework region FR3 |
| SEQ ID NO: 57 | Framework region FR4 |
| SEQ ID NO: 58 | Framework region FR5 |
| SEQ ID NO: 59 | Framework region FR6 |
| SEQ ID NO: 60 | Framework region FR7 |
| SEQ ID NO: 61 | Framework region FR8 |

Nucleic acid sequences:

| | |
|---|---|
| SEQ ID NO: 37 | DNA sequence encoding HC of mAb1-M and |
| | of mAb2-M (HC-LALA-YTE) |
| SEQ ID NO: 38 | DNA sequence encoding HC of mAb3-M |
| | (LC-K222R-LALA-YTE) |
| SEQ ID NO: 39 | DNA sequence encoding LC of mAb2-M, of |
| | mAb3-M and of mAb7-M (LC-Tag) |
| SEQ ID NO: 42 | DNA sequence encoding HC of mAb5-M |
| | (antiCD20-HC) |
| SEQ ID NO: 43 | DNA sequence encoding LC of mAb5-M |
| | (antiCD20-LC) |
| SEQ ID NO: 46 | DNA sequence encoding Transglutaminase |
| SEQ ID NO: 52 | DNA sequence encoding HC of mAb6-M and |
| | of mAb7-M (HC-LALA) |
| SEQ ID NO: 53 | DNA sequence encoding LC of mAb1-M and |
| | of mAb6-M |

Amino acid sequences:

| | |
|---|---|
| SEQ ID NO: 62 | labetuzumab heavy chain |
| SEQ ID NO: 63 | labetuzumab light chain |
| SEQ ID NO: 64 | rituximab HC |
| SEQ ID NO: 65 | rituximab LC |
| SEQ ID NO: 66 | mAb_tag2_LC |
| SEQ ID NO: 67 | mAb_tag2_HC (HC-tag) |
| SEQ ID NO: 68 | mAb_tag3_LC (LC-tag1) |
| SEQ ID NO: 69 | mAb_tag4_LC (LC-tag2) |
| SEQ ID NO: 70 | mAb_tag3&4_HC (HC) |
| SEQ ID NO: 71 | mAb_tag5_LC (LC-tag) |
| SEQ ID NO: 72 | mAb 7-M_HC (ceacam-HC) |
| SEQ ID NO: 73 | Transglutaminase (activated, version2 from |
| | S. ladakanum) |
| SEQ ID NO: 74 | mTG tag GGTLQSPP |
| SEQ ID NO: 77 | mTG tag TLQSG |
| SEQ ID NO: 78 | mTG tag TLQSPP |
| SEQ ID NO: 79 | mTG tag GGTLQSG |
| SEQ ID NO: 80 | mTG tag TLQSA |
| SEQ ID NO: 81 | MTG_5 (from S. mobaraensis) |
| SEQ ID NO: 82 | MTG_6 (from S. mobaraensis) |
| SEQ ID NO: 83 | MTG_7 (from S. mobaraensis) |
| SEQ ID NO: 84 | MTG_8 (from S. mobaraensis) |

### Example 1: Synthesis of a Drug-Linker Compound with Glucuronide-Based Linker: Drug-Linker Compound 1-M (DL1-M)

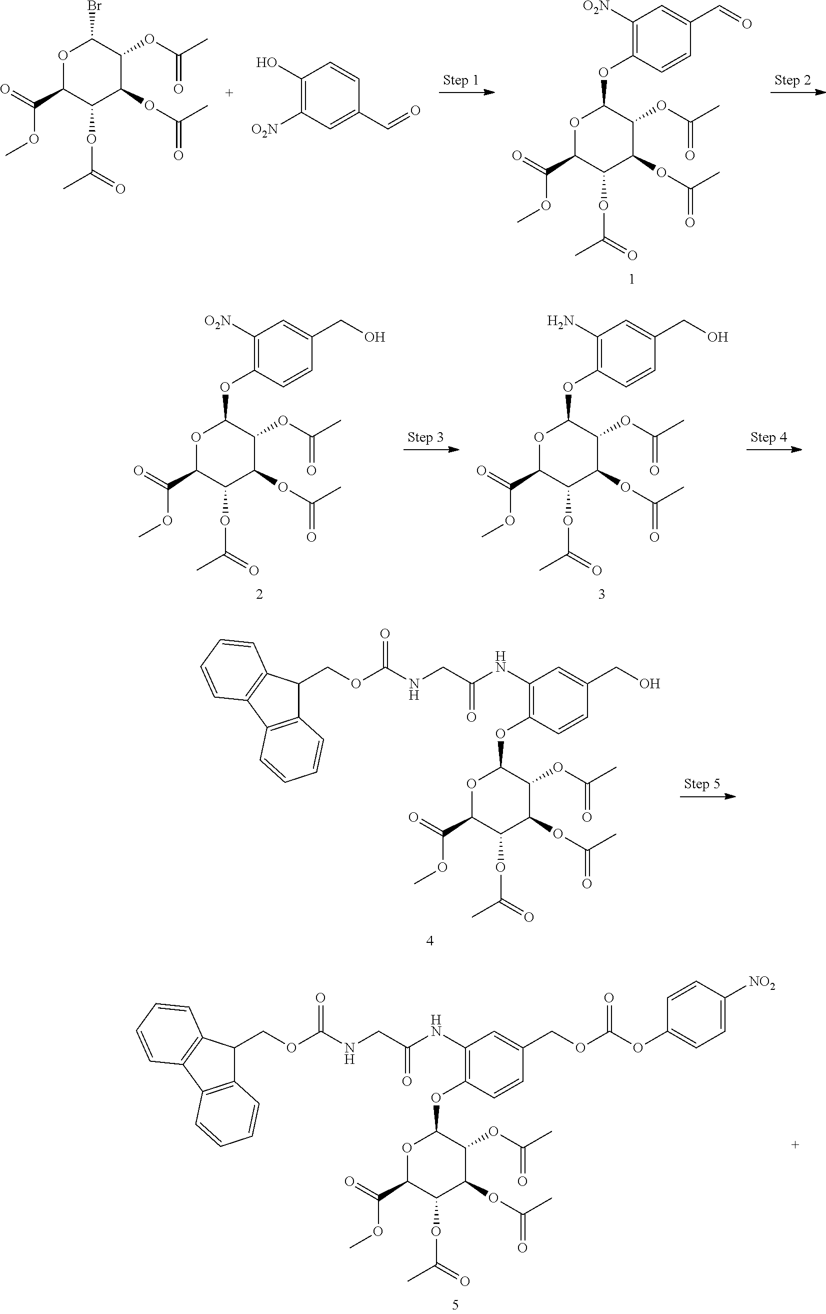

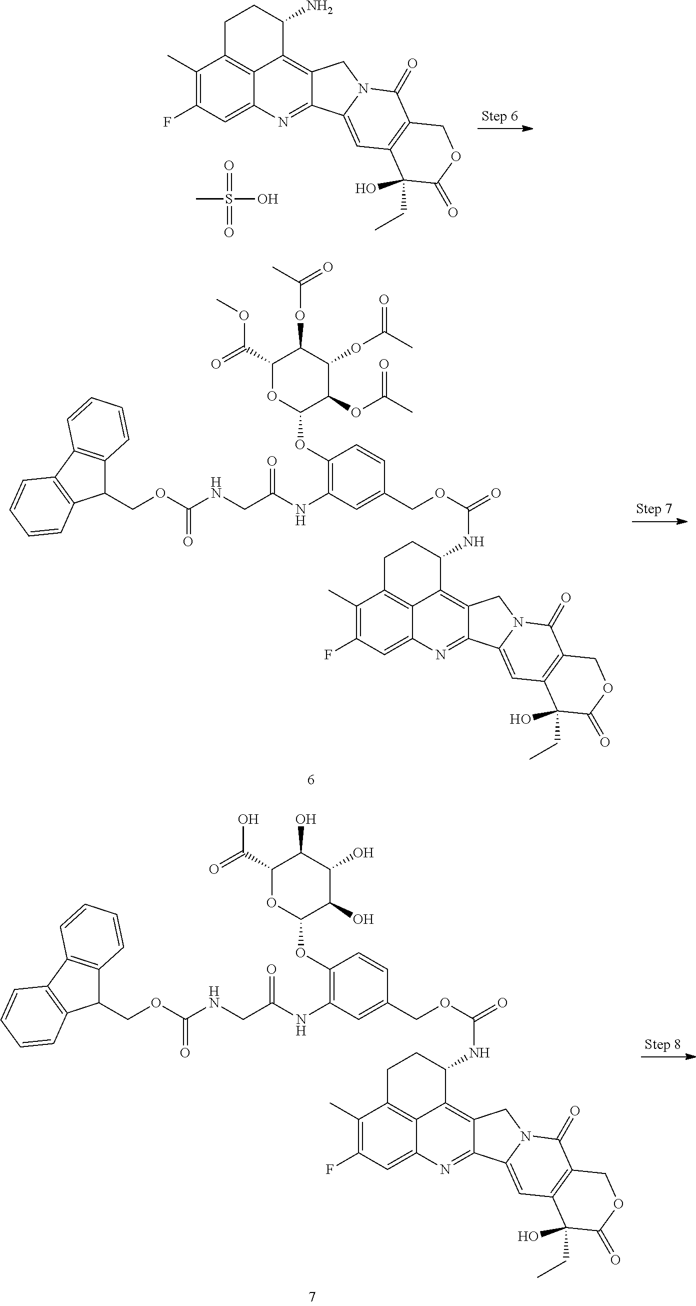

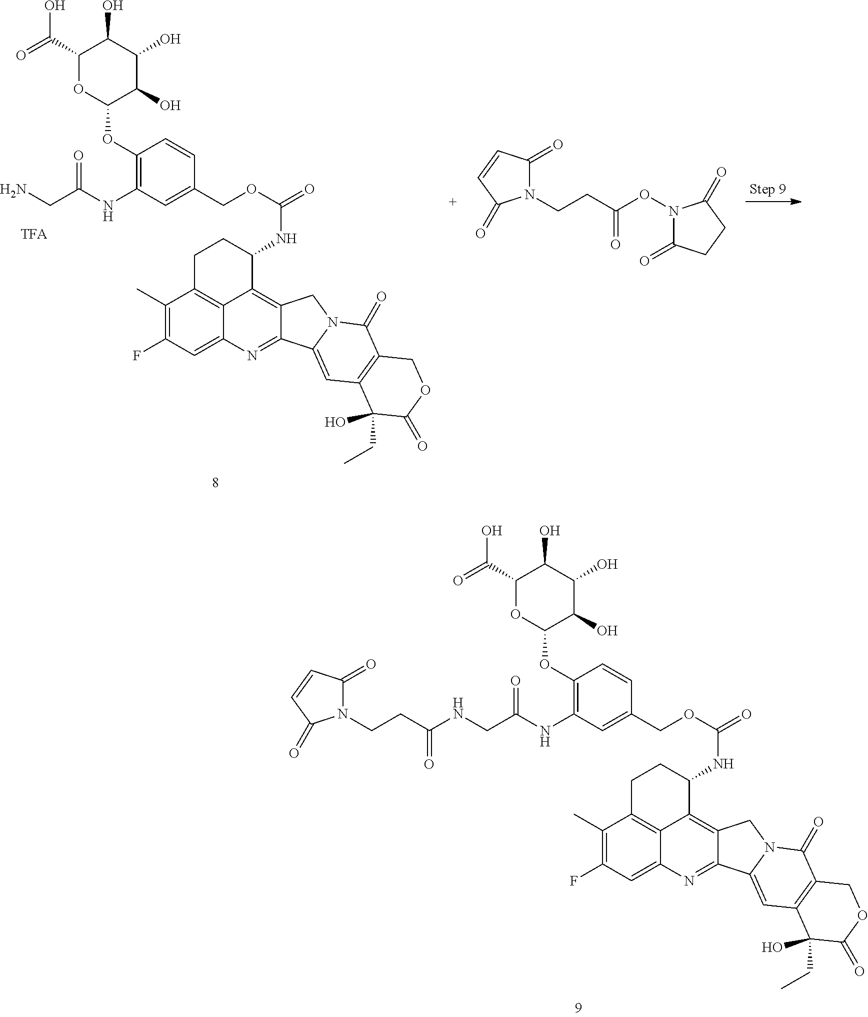 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3 ; O=C(CCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CCN2C(=O)C=CC2=O)c1)CC3]

The synthetic route to compound 9).

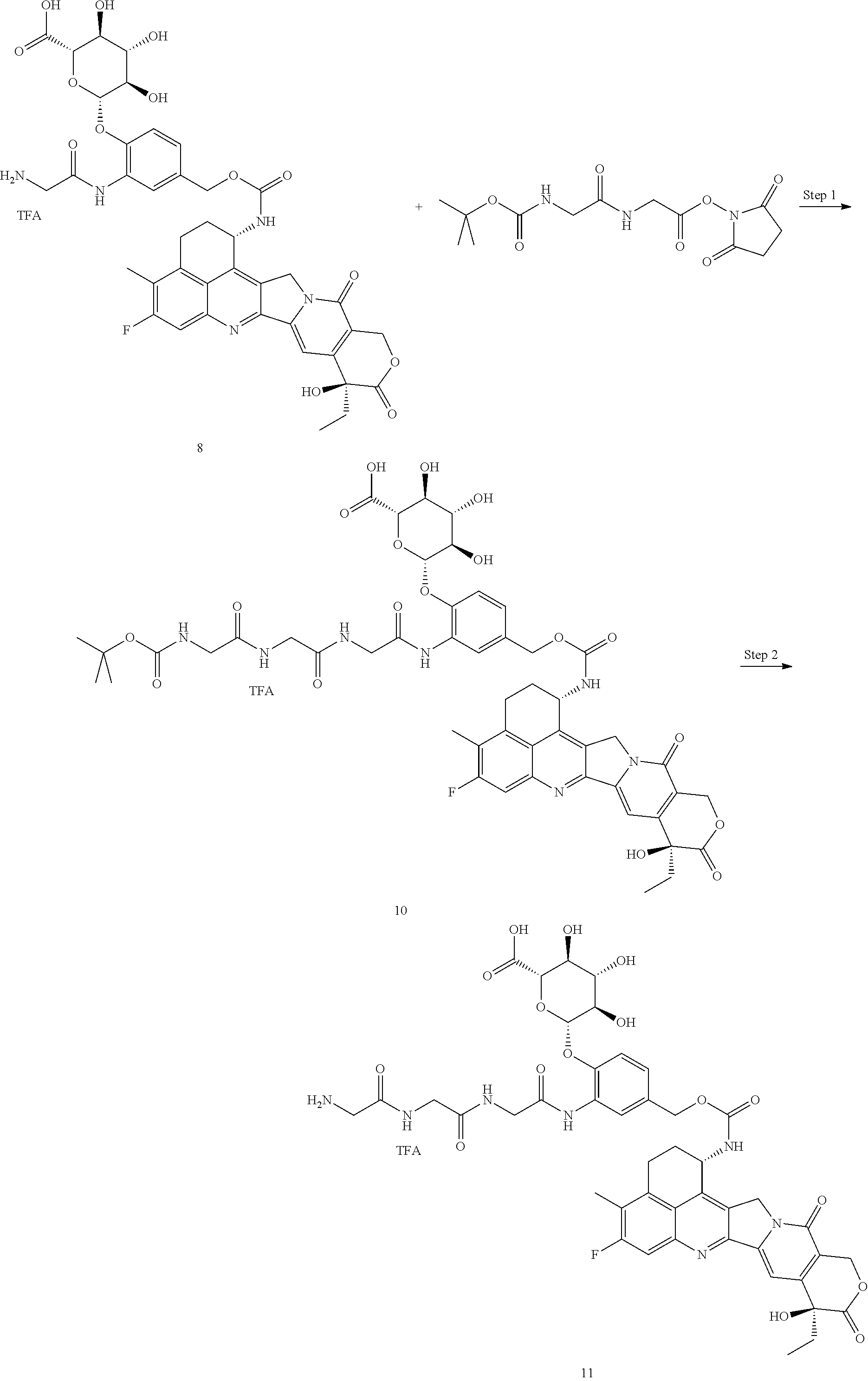

The synthetic route to compound 11 (also referred to herein as drug-linker compound 1-M (DL1-M)

### Protocol of Chemical Preparation to Compound 9

### Step 1: Compound 1

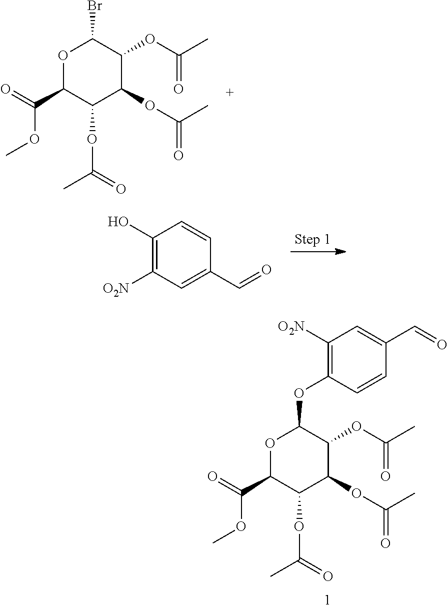

To a stirred solution of (2S,3S,4S,5R,6R)-3,4,5-Triacetoxy-6-bromo-tetrahydro-pyran-2-carboxylic acid methyl ester (8.30 g; 20.90 mmol; 1.00 eq.) and 4-Hydroxy-3-nitro-benzaldehyde (5.24 g; 31.35 mmol; 1.50 eq.) in Acetonitrile (83.00 ml; 10.00 V) was added Silver (I) oxide (9.69 g; 41.80 mmol; 2.00 eq.). The reaction mixture was stirred at RT for 16 h. The reaction mixture was filtered through celite. The filtrate was concentrated under vacuum to get solid. The solid was dissolved in EtOAc and washed with 10% aqueous solution of NaHCO₃ to remove excess 4-Hydroxy-3-nitro-benzaldehyde. The organic layer was concentrated under vacuum to get compound 1 as sand colour solid.
Yield: 9.0 g
Percentage Yield: 89.1%
Analytical data:
NMR: ¹H-NMR (400 MHZ, DMSO-d₆): 9.98 (s, 1H), 8.46 (s, 1H), 8.25-8.21 (m, 1H), 7.64 (d, J=11.60 Hz, 1H), 5.94 (d, J=10.00 Hz, 1H), 5.51-5.44 (m, 1H), 5.20-5.09 (m, 2H), 4.80 (d, J=13.20 Hz, 1H), 3.64 (s, 3H), 2.09 (s, 9H).

### Step 2: Compound 2

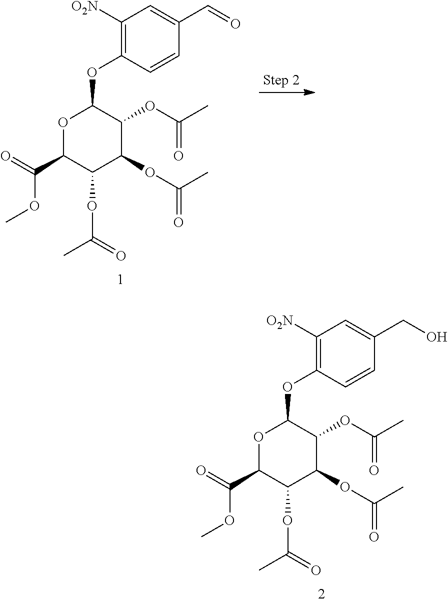 [SMILES: COC(=O)[C@H]1O[C@@H](Oc2ccc(C=O)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2[N+](=O)[O-])[C@H](OC(C)=O)[C@@H](OC(C)=O)[C@@H]1OC(C)=O]

To a stirred solution of compound 1 (9.00 g; 18.62 mmol; 1.00 eq.) in Propan-2-ol (33.00 ml; 3.67 V) and CHCl₃ (167.00 ml; 18.56 V) were added silica gel 60-120 (3.60 g; 112.09 mmol; 6.02 eq.) followed by sodium borohydride (1.80 g; 46.55 mmol; 2.50 eq.). The reaction mixture was stirred for 1 h at RT. After completion, the reaction mixture was quenched with cooled H₂O and filtered through celite. The filtrate was extracted with Dichloromethane and dried over Na₂SO₄. The solvent was concentrated to get compound 2 as off-white powder.
Yield: 8.70 g
Percentage Yield: 92.4%
Analytical data
LCMS: Column: ATLANTIS dC18 (50×4.6 mm) 5 μm; Mobile phase A: 0.1% HCOOH in H₂O: ACN (95:5); B: ACN
RT (min): 2.05; M+H: 503.2, Purity: 96.6%

### Step 3: Compound 3

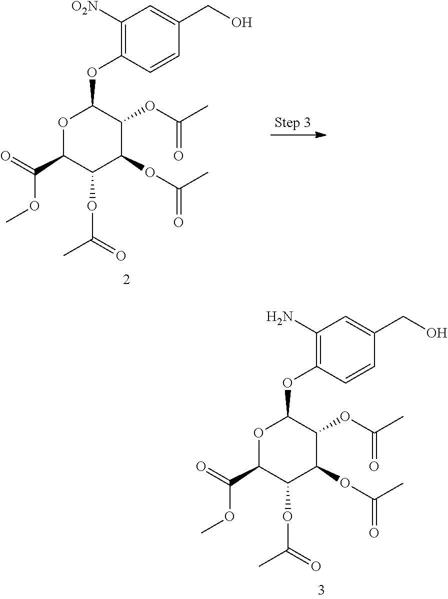 [SMILES: COC(=O)[C@H]1O[C@@H](Oc2ccc(CO)cc2[N+](=O)[O-])[C@H](OC(C)=O)[C@@H](OC(C)=O)[C@@H]1OC(C)=O ; COC(=O)[C@H]1O[C@@H](Oc2ccc(CO)cc2N)[C@H](OC(C)=O)[C@@H](OC(C)=O)[C@@H]1OC(C)=O]

To a stirred solution of compound 2 (8.70 g; 17.21 mmol; 1.00 eq.) in ethyl acetate (100.00 ml; 11.49 V) and THF (100.00 ml; 11.49 V) was added Palladium on carbon (10% w/w) (2.50 g; 2.35 mmol; 0.14 eq.). The reaction mixture stirred for 3 h at RT under hydrogen atmosphere. After completion, the reaction mixture was filtered off through celite. The solvent was concentrated under vacuum to get compound 3 as off-white solid.
Yield: 8.5 g
Percentage Yield: 100%
Analytical data:
LCMS: Column: ATLANTIS dC18 (50×4.6 mm) 5 μm; Mobile phase A: 0.1% HCOOH in H₂O: ACN (95:5); B: ACN
RT (min): 1.73; M+H: 456.10, Purity: 95.1%

### Step 4: Compound 4

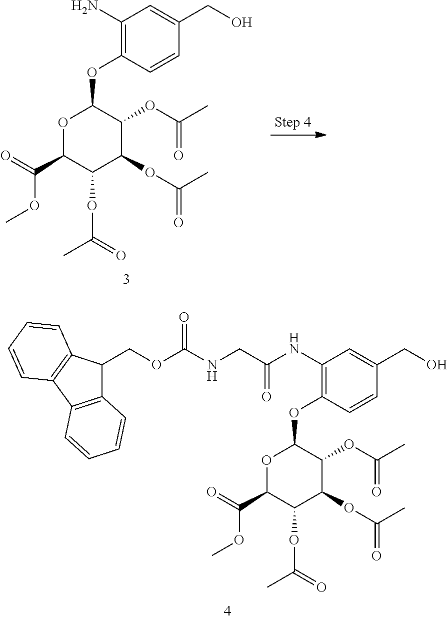 [SMILES: COC(=O)[C@H]1O[C@@H](Oc2ccc(CO)cc2N)[C@H](OC(C)=O)[C@@H](OC(C)=O)[C@@H]1OC(C)=O ; COC(=O)[C@H]1O[C@@H](Oc2ccc(CO)cc2NC(=O)CNC(=O)OCC2c3ccccc3-c3ccccc32)[C@H](OC(C)=O)[C@@H](OC(C)=O)[C@@H]1OC(C)=O]

To a stirred solution of compound 3 (10.00 g; 20.89 mmol; 1.00 eq.) and (9H-Fluoren-9-ylmethoxycarbonylamino)-acetic acid (7.60 g; 25.06 mmol; 1.20 eq.) in DCM (250.00 ml; 25.00 V) was added 2-Ethoxy-2H-quinoline-1-carboxylic acid ethyl ester (15.65 g; 62.66 mmol; 3.00 eq.) at 0° C. The reaction mixture was stirred for 16 h at RT. After completion, solvent was removed under reduced pressure to get a crude product. The crude product was purified by column chromatography (56% EtOAc: petroleum ether) to get compound with purity 80%. The compound was purified further by washings with 30% EtOAc and pet ether to get compound 4 as white solid.
Yield: 8.5 g
Percentage Yield: 50.7%
Analytical data:
LCMS: Column: ATLANTIS dC18 (50×4.6 mm) 5 μm; Mobile phase A: 0.1% HCOOH in H₂O: ACN (95:5); B: ACN
RT (min): 3.03; M+H: 735.2, Purity: 81.9%

### Step 5: Compound 5

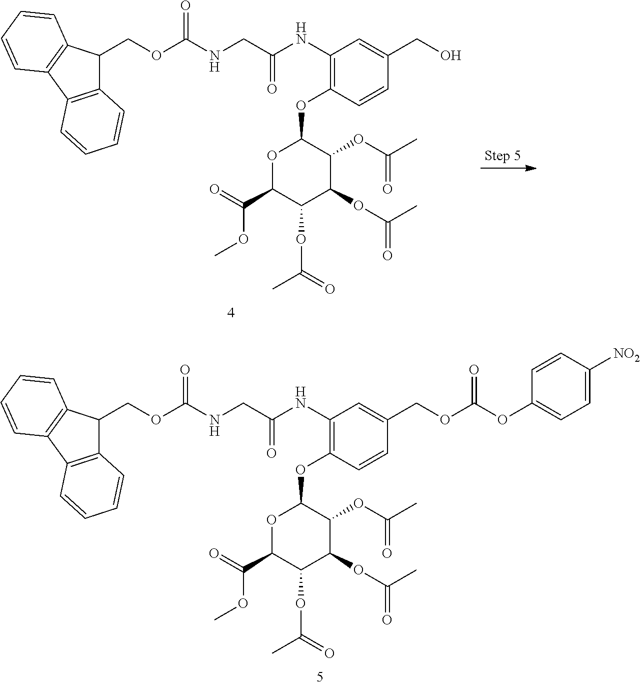 [SMILES: COC(=O)[C@H]1O[C@@H](Oc2ccc(CO)cc2NC(=O)CNC(=O)OCC2c3ccccc3-c3ccccc32)[C@H](OC(C)=O)[C@@H](OC(C)=O)[C@@H]1OC(C)=O ; COC(=O)[C@H]1O[C@@H](Oc2ccc(COC(=O)Oc3ccc([N+](=O)[O-])cc3)cc2NC(=O)CNC(=O)OCC2c3ccccc3-c3ccccc32)[C@H](OC(C)=O)[C@@H](OC(C)=O)[C@@H]1OC(C)=O]

To a stirred solution of compound 4 (2.00 g; 2.49 mmol; 1.00 eq.) in THF (40.00 ml; 20.00 V) at 0° C., were added Carbonic acid bis-(4-nitro-phenyl) ester (3.06 g; 9.97 mmol; 4.00 eq.) and DIPEA (4.40 ml; 24.92 mmol; 10.00 eq.). The reaction mixture was stirred at RT for 12 h. After completion of the reaction, reaction mixture was concentrated under vacuum. The crude product was purified by column chromatography using silica gel (230-400) and pet ether/ethyl acetate as an eluent to afford compound 5 as pale yellow solid.
Yield: 2.0 g
Percentage Yield: 84.6%
Analytical data:
LCMS: Column: X-Bridge C8 (50λ4.6) mm, 3.5 μm; Mobile phase: A: 0.1% TFA in MilliQ water; B: ACN
RT (min): 3.24; M+H: 900.20, Purity: 94.9%

### Step 6: Compound 6

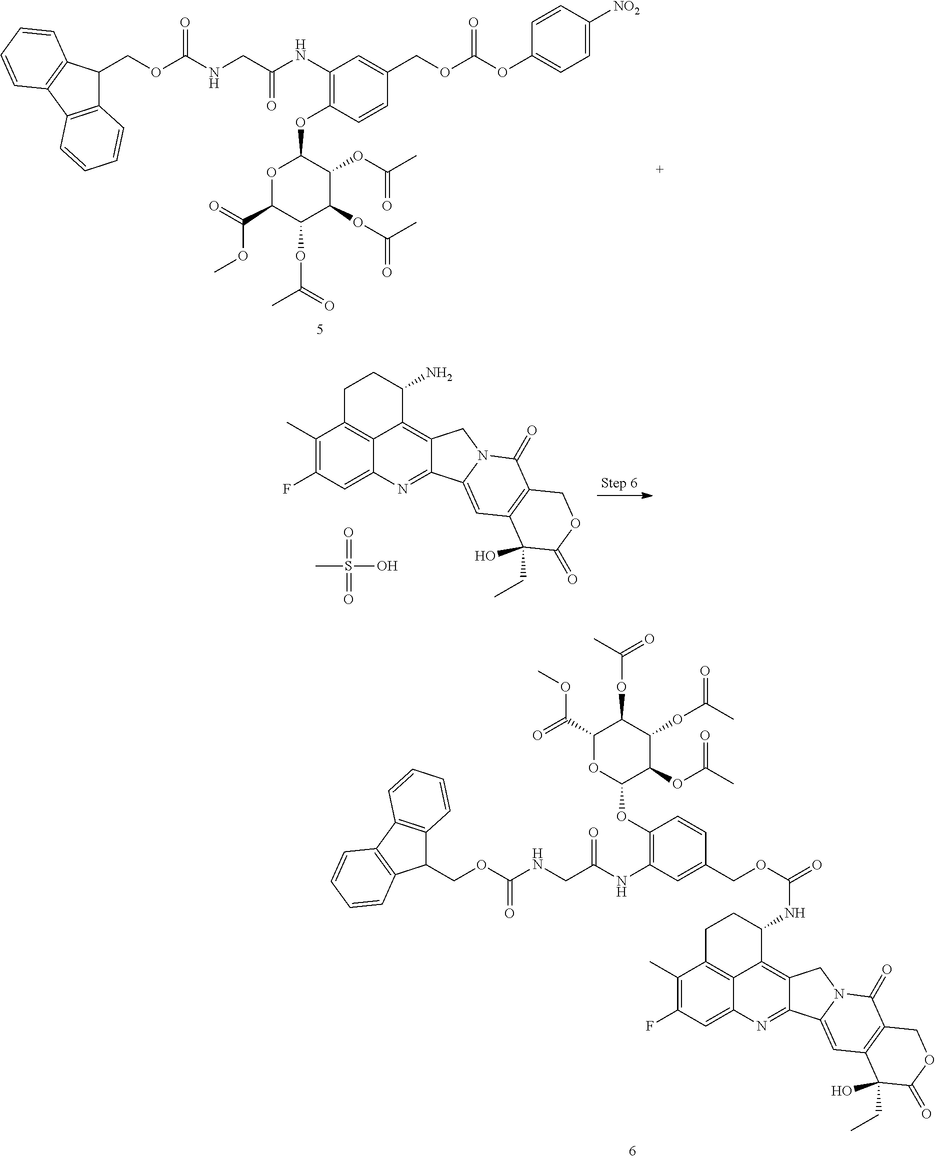

Compound 5 (1,369 g; 1,00 eq.) was dissolved in N,N-dimethylformamide (15.00 ml), Exatecan mesylate (679.7 mg; 1,00 eq.), 4-methylmorpholine for synthesis (0,422 ml; 3,00 eq.) and 1-Hydroxybenzotriazol (172.8 mg; 1,00 eq.) were added. The reaction mixture was stirred at room temperature for overnight. After the stirring time the reaction suspension was changed to a brown solution. The reaction was monitored by LC-MS, which showed a complete conversion of the starting material. The reaction mixture was purified via RP flash chromatography. The product containing fractions were combined, concentrated in vacuo and lyophilized overnight to afford compound 6 as an yellow solid.
Yield: 1.59 g
Percentage Yield: 87.5%
Analytical data:
LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min
RT (min): 1.95; M+H: 1196.40, Purity: 84.4%.

### Step 7: Compound 7

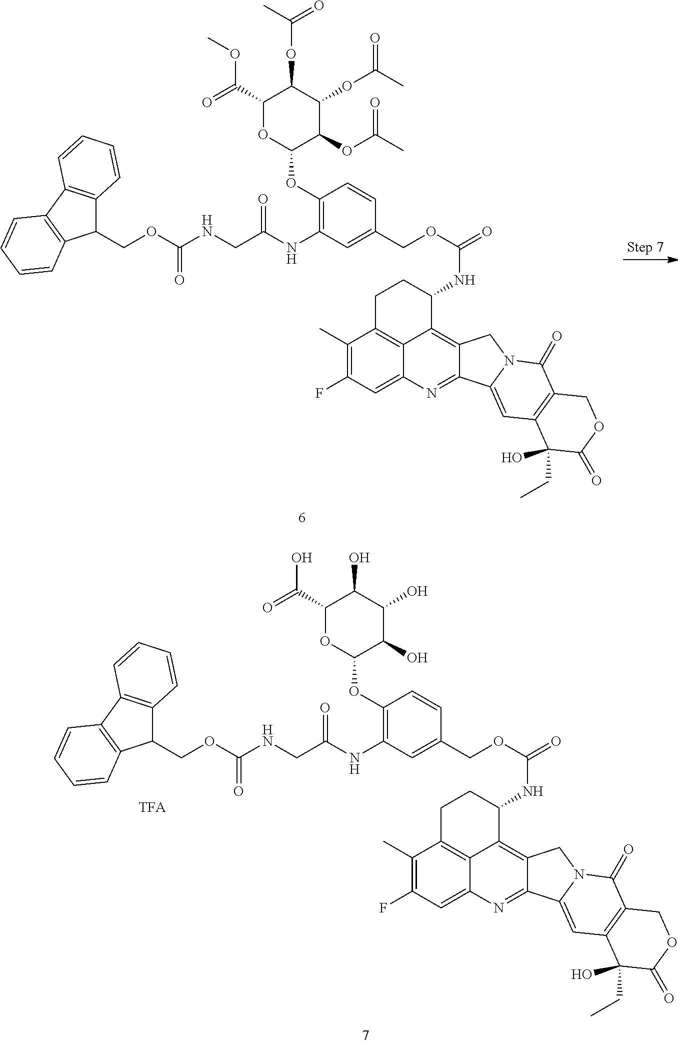 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)OC)[C@@H](OC(C)=O)[C@H](OC(C)=O)[C@H]2OC(C)=O)c(NC(=O)CNC(=O)OCC2c4ccccc4-c4ccccc42)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)OCC2c4ccccc4-c4ccccc42)c1)CC3]

Compound 6 (1,586 g; 1,00 eq.) was dissolved in tetrahydrofuran (50.00 ml) and a solution (0.1M) of LiOH (contains Lithium hydroxide hydrate (281.77 mg; 6,00 eq.) in water (67,100 ml)) was added dropwise at 0° C. The pH value was checked during the addition. The pH should not exceed 10. The addition of the solution of LiOH was completed after 1.5 hours. The reaction was monitored by LC-MS, which showed a complete conversion of the starting material. The reaction was quenched with citric acid solution, pH adjusted to 5. The reaction mixture was concentrated under reduced pressure. The crude was purified by prep. HPLC. The product containing fractions were combined and lyophilized to afford Compound 7 as a dark yellow solid.
Yield: 728 mg
Percentage Yield: 54.8%
Analytical data:
LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min
RT (min): 1.68; M+H: 1056.30, Purity: 98.5%.

### Step 8: Compound 8

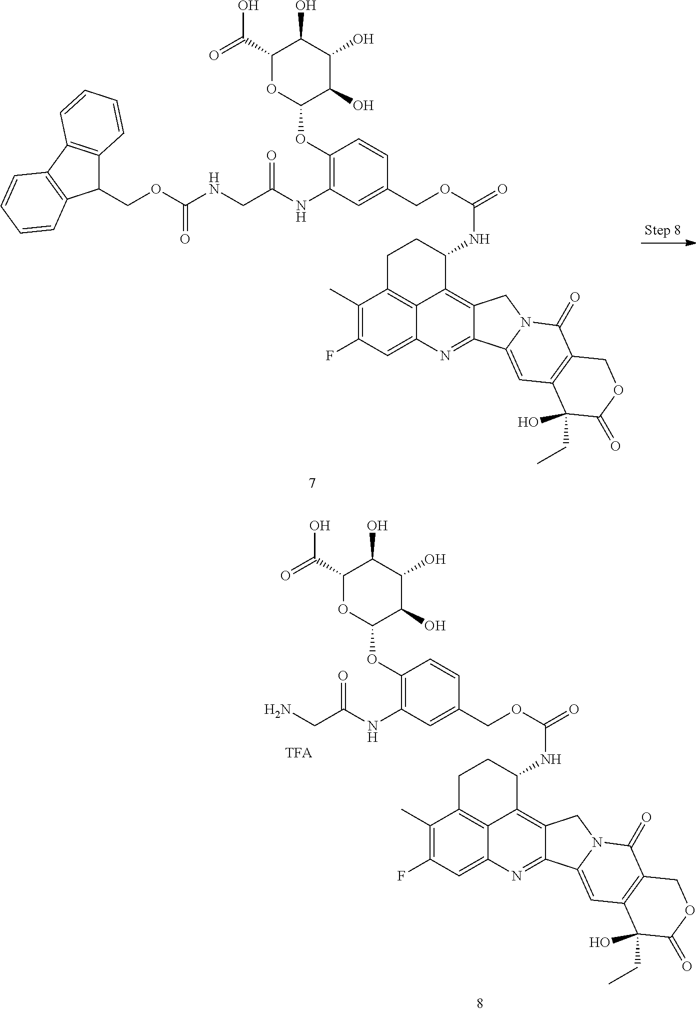 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)OCC2c4ccccc4-c4ccccc42)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3]

Compound 7 (728,000 mg; 1,00 eq.) was dissolved in N,N-dimethylformamide (20.00 ml). Piperidine (136,513 μl; 2,00 eq.) was added and the solution was stirred at RT for totally 4 hours. The reaction was monitored by LC-MS, which showed a complete conversion of the starting material. The reaction mixture was concentrated under reduced pressure and the crude product was purified by RP flash chromatography. The product containing fractions were combined, the solvent was removed partially and it was lyophilized overnight to afford compound 8 as an yellow solid.
Yield: 706 mg
Percentage Yield: 100%
Analytical data:
LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min
RT (min): 1.22; M+H: 834.30, Purity: 97.6%.

### Step 9: Compound 9

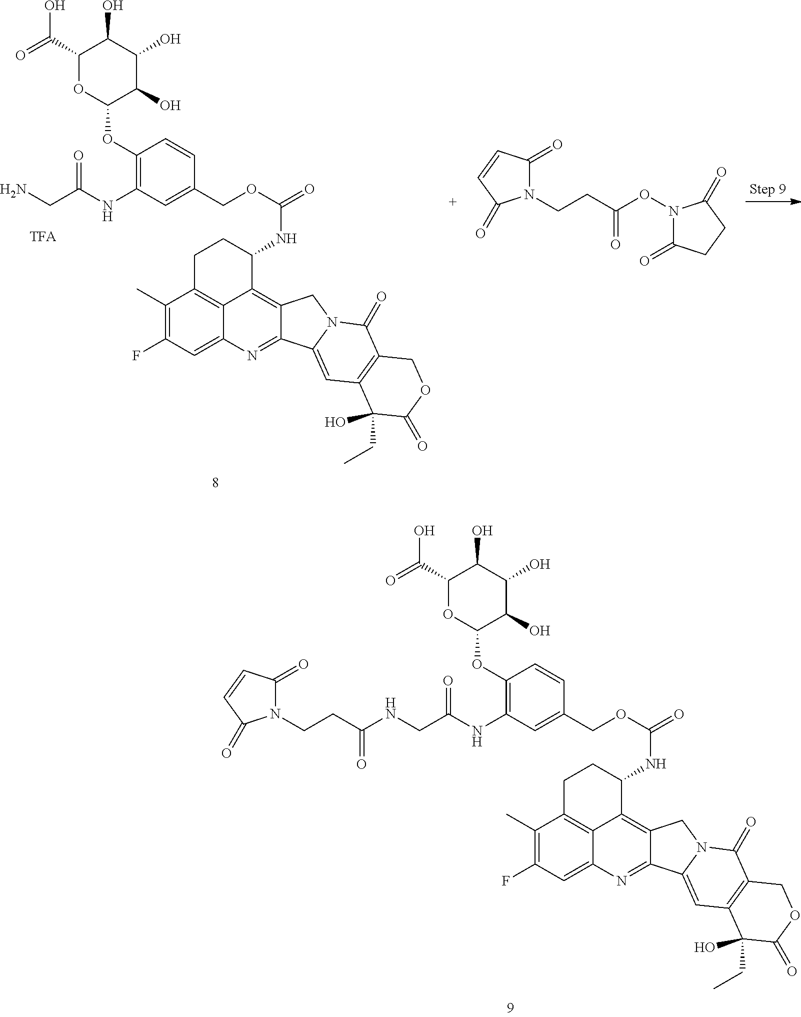 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3 ; O=C(CCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CCN2C(=O)C=CC2=O)c1)CC3]

To a solution of compound 8 (854 mg; 1,00 eq.) in dimethylformamid (30.00 ml) were added N-ethyldiisopropylamine (149,234 μl; 1,00 eq.) and 3-(2,5-Dioxo-2,5-dihydro-pyrrol-1-yl)-propionic acid 2,5-dioxo-pyrrolidin-1-yl ester (233.61 mg; 1,00 eq.). The reaction mixture was stirred at RT for 3 hours. The reaction was monitored by LC-MS, which showed a complete conversion of the starting material. The reaction mixture was concentrated under reduced pressure and the crude product was by RP flash chromatography. The product containing fractions were combined, concentrated and lyophilized to give the desired produce with a purity of 91%. This material was again purified by RP chromatography to give compound 9 as an yellow solid.
Yield: 580 mg
Percentage Yield: 60.1%
Analytical data:

LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min

RT (min): 1.38; M+H: 985.30, Purity: 90% (the other 10% of isomer can be removed by HPLC)

¹H NMR (500 MHZ, DMSO-d₆) δ 13.10-12.44 (m, 1H), 9.08 (s, 1H), 8.32 (t, J=5.8 Hz, 1H), 8.16 (s, 1H), 8.02 (d, J=8.8 Hz, 1H), 7.76 (d, J=10.9 Hz, 1H), 7.31 (s, 1H), 7.15-7.09 (m, 2H), 6.98 (s, 2H), 5.48-5.38 (m, 2H), 5.32-5.22 (m, 3H), 5.11-5.01 (m, 2H), 4.87 (d, J=7.6 Hz, 1H), 3.92-3.88 (m, 1H), 3.89-3.84 (m, 2H), 3.65-3.61 (m, 2H), 3.46-3.41 (m, 1H), 3.42-3.37 (m, 1H), 3.38-3.31 (m, 1H), 3.28-3.20 (m, 1H), 3.15-3.07 (m, 1H), 2.48-2.44 (m, 2H), 2.38 (s, 3H), 2.24-2.13 (m, 2H), 1.94-1.80 (m, 2H), 0.88 (t, J=7.3 Hz, 3H).

### Protocol of Chemical Preparation to Compound 11

### Step 1: Compound 10

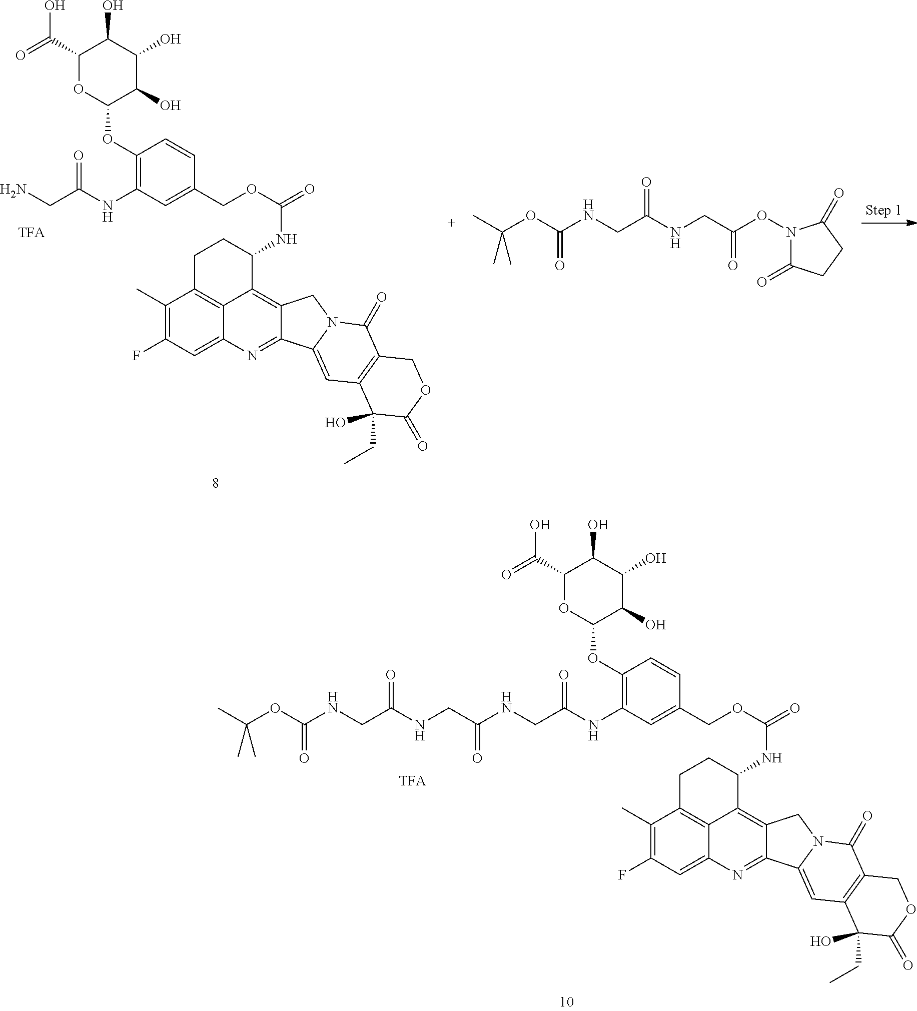 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CN)c1)CC3 ; CC(C)(C)OC(=O)NCC(=O)NCC(=O)ON1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CNC(=O)OC(C)(C)C)c1)CC3]

To a solution of compound 8 (289.00 mg; 1.00 eq) in dimethyl formamide (10.00 ml) were added N-ethyl diisopropylamino (0.104 ml, 2.00 eq) and 2,5-dioxopyrrolidin-1-yl (tert-butoxycarbonyl) glycylglycinate (105.70 mg; 1.00 eq). The reaction mixture was stirred at room temperature for 1 hour and the reaction was mirrored by LC-MS. After completion, the solvent was removed in vacuo and the crude product was purified by prep. HPLC over a Sunfire column. The fractions containing product were combined and lyophilized to afford compound 10 trifluoroacetic acid salt as a yellow solid.
Yield: 162.00 mg, 0.14 mmol.
Percentage yield: 44.8%
Analytical data:

LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min RT (min): 1.41; M+H: 1048.1, Purity: 97.9%

### Step 2: Compound 11

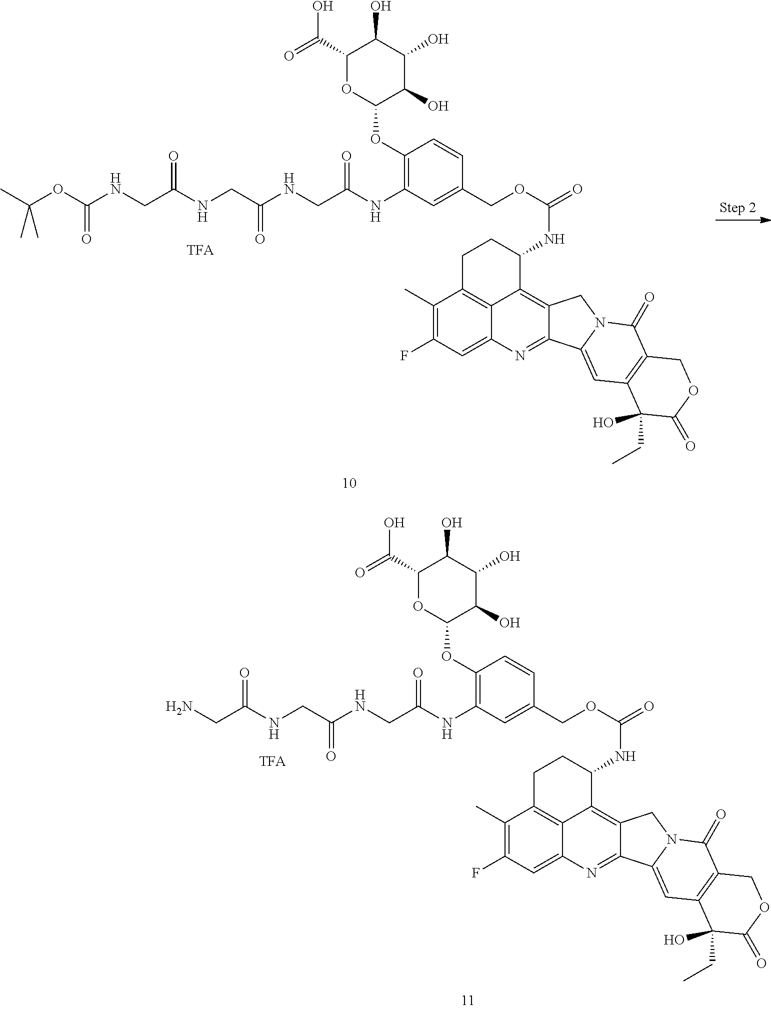 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CNC(=O)OC(C)(C)C)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CN)c1)CC3]

To a suspension of compound 10 (162.00 mg; 1.00 eq) in dichloromethane (5.00 ml) was added 4.0 M hydrogen chloride solution in 1,4-dioxane (682.43 μl; 20.00 eq) and it was stirred at room temperature for 3.5 hours and the reaction was monitored by LC-MS. After completion, the reaction mixture was concentrated in vacuo and the crude product was purified by prep. HPLC. The fractions containing product were combined and lyophilized to afford compound 11 trifluoroacetic acid salt as a yellow solid.
Yield: 113 mg; 0.11 mmol.
Percent yield: 77.0%
Analytical data:

LCMS: Column: Chromolith HR RP-18e (50-4.6 mm); Mobile phase A: 0.05% HCOOH in H₂O; B: 0.04% HCOOH and 1% H₂O in ACN; T: 40° C.; Flow: 3.3 ml/min; MS: 100-2000, amu positive; 1%->100% B: 0->2.0 min; 100% B: 2,0->2.5 min

RT (min): 1.22; M+H: 948.0, M+2H: 474.8; Purity: 98.8%

¹H NMR (700 MHZ, DMSO-d₆) δ 12.93-12.79 (m, 1H), 9.14 (s, 1H), 8.61 (t, J=5.8 Hz, 1H), 8.41 (t, J=5.9 Hz, 1H), 8.14 (s, 1H), 8.05 (d, J=8.8 Hz, 1H), 8.03-7.93 (m, 3H), 7.79 (d, J=10.8 Hz, 1H), 7.31 (s, 1H), 7.13 (s, 2H), 6.58-6.47 (m, 1H), 5.80-5.61 (m, 1H), 5.48-5.40 (m, 2H), 5.31-5.24 (m, 3H), 5.08 (d, J=12.2 Hz, 1H), 5.03 (d, J=12.3 Hz, 1H), 4.91 (d, J=7.7 Hz, 1H), 3.96 (d, J=5.8 Hz, 2H), 3.95-3.86 (m, 3H), 3.62 (q, J=5.8 Hz, 2H), 3.43 (t, J=9.3 Hz, 1H), 3.41 (t, J=8.5 Hz, 1H), 3.35 (t, J=9.0 Hz, 1H), 3.27-3.20 (m, 1H), 3.15-3.09 (m, 1H), 2.38 (s, 3H), 2.23-2.13 (m, 2H), 1.93-1.81 (m, 2H), 0.88 (t, J=7.3 Hz, 3H).

### Example 2: Expression and Purification of Exemplary Modified Antibodies and of Transglutaminase

In the following, the expression and purification of exemplary modified antibodies will be described. These exemplary modified antibodies can be used for the production of a respective antibody-drug-conjugate using the method of the invention. The features of the modified antibodies mAb1-M, mAb2-M, mAb3-M, mAb4-M, mAb5-M, mAb6-M and mAb7-M are outlined in the following Table 4:

TABLE 4

| | | | Amino acid |
|---|---|---|---|
| | | | sequence for |
| | | Antibody-drug | heavy chain |
| | | conjugate features, | (HC) and light |
| Antibody/Antibody- | | incl. preferred drug- | chain (LC) of |
| drug conjugate | | to-antibody ratio, | the mAb |
| name | Antibody features | linker type and drug | antibody |
| mAb1-M, | anti-CEACAM5 mAb | interchain cysteine | HC = SEQ ID |
| ADC1-M | LALA mutation | conjugation | NO: 34 |
| | YTE mutation | DAR = 8 | LC = SEQ ID |
| | | maleimido-b- | NO: 14 |
| | | glucuronide-exatecan | |
| | | (DL1) | |
| mAb2-M, | anti-CEACAM5 mAb | MTG coupling | HC = SEQ ID |
| ADC2-M | LALA mutation | DAR = 4 | NO: 34 |
| | YTE mutation | NH2-GGG-b- | LC = SEQ ID |
| | MTG Tag | glucuronide-exatecan | NO: 36 |
| | | (DL1-M) | |
| mAb3-M, | anti-CEACAM5 mAb | MTG coupling | HC = SEQ ID |
| ADC3-M | LALA mutation | DAR = 4 | NO: 35 |
| | YTE mutation | NH2-GGG-b- | LC = SEQ ID |
| | K222R mutation (MTG | glucuronide-exatecan | NO: 36 |
| | optimization) | (DL1-M) | |
| | MTG Tag | | |
| mAb4-M, | anti-CD20 Rituximab | interchain cysteine | HC = SEQ ID |
| ADC4-M | based isotype control | conjugation | NO: 64 |
| | | DAR = 8 | LC = SEQ ID |
| | | maleimido-b- | NO: 65 |
| | | glucuronide-exatecan | (Rituximab |
| | | (DL1) | mAb) |
| mAb5-M, | anti-CD20 Rituximab | MTG coupling | HC = SEQ ID |
| ADC5-M | based isotype control | DAR = 4 | NO: 40 |
| | LALA mutation | NH2-GGG-b- | LC = SEQ ID |
| | MTG Tag | glucuronide-exatecan | NO: 41 |
| | | (DL1-M) | |
| mAb6-M, | anti-CEACAM5 mAb | interchain cysteine | HC = SEQ ID |
| ADC6-M | LALA mutation | conjugation | NO: 51 |
| | | DAR = 8 | LC = SEQ ID |
| | | maleimido b- | NO: 14 |
| | | glucuronide-exatecan | |
| | | (DL1) | |
| mAb7-M, | anti-CEACAM5 mAb | MTG coupling | HC = SEQ ID |
| ADC7-M | LALA mutation | DAR = 4 | NO: 51 |
| | MTG Tag | NH2-GGG-b- | LC = SEQ ID |
| | | glucuronide-exatecan | NO: 36 |
| | | (DL1-M) | |

DNA sequences encoding for antibodies mAb1-M, mAb2-M, mAb3-M, mAb5-M, mAb6-M and mAb7-M were synthesized and cloned onto pTT5 plasmids for recombinant expression at GeneArt (Life Technologies). Produced plasmids were used for transient transfection and recombinant protein expression in shaking flasks using the ExpiCHO expression system (Gibco™, Thermo Fisher Scientific Inc.). Seven days post transfection, supernatants were harvested and expressed antibodies purified using a standard stepwise process including protein A affinity chromatography (HiTrap MabSelect SuRe columns, Cytiva) and size-exclusion chromatography (HiLoad Superdex 200 pg columns, Cytiva).

mAb4-M/Rituximab (F. Hoffmann-La Roche Ltd., Basel, Switzerland) was purchased in pharmacy grade quality.

### Expression and Purification of Transglutaminase

A DNA sequence encoding for transglutaminase enzyme mTG (Seq ID 46) was synthesized and cloned onto a pET30a plasmid for recombinant expression at GeneArt (Life Technologies). An Escherichia coli BL21 (DE3) strain transformed with the generated plasmid was cultivated in shaking flasks in lysogenic broth medium supplemented with, 5 g/l glucose, 10 ml/100 ml 10× phosphate buffered saline and 30 mg/l kanamycin overnight at 28° C. and 130 rpm (50 mm throw). This culture was used to inoculate a fermenter containing 9.5 I liter growth medium (50 g/l yeast extract, 10 g/l peptone, 0.5 g/l MgSO₄×7 H₂O and 2 ml 50% Desmophen (antifoam by Rhein Chemie Rheinau) to an optical density of 0.00002. The fermenter was run at 28° C. with 800 U/min revolutions, 5 NI/min aeration and pH 7.0-7.4 over night (16 h). At OD 5 the culture was induced with 0.1 mM IPTG until an OD of ˜30 was reached (5-6 hours). In case of foam formation or a drop in oxygen concentration below 2 mg/ml, more Desmophen was added, or the revolutions increased to 1000 rpm, respectively. The cell mass was harvested by continuous flow-through centrifugation. 50 g of cell pellet were resuspended in 250 ml 50 mM Na-acetate, 1 mM DTT, 5 mM MgCl₂, 25 U/ml Benzonase (Millipore) pH 5.5 and disrupted using a French Press. The supernatant was clarified by centrifugation and filtration (0.8/0.2 μm pore size), pH adjusted to 5.5 and loaded onto a Fractogel® SO₃-(M) 85 ml column (Millipore) followed elution with a linear gradient of 20 CV from 0-1 M NaCl. Fractions with purified protein of expected size were identified by SDS-PAGE, pooled and dialyzed overnight at 4° C. against 50 mM Tris/HCl, 300 mM NaCl, pH 8.0. Transglutaminase was then proteolytic processed at a final concentration of 2 mg/ml using 0.5 U/ml Dispase®| (Sigma Aldrich) and 2 mM CaCl₂) for 60 min at 37° C. followed by addition of 5 mM EDTA. The reaction mix was dialyzed overnight at 4° C. against 50 mM sodium phosphate buffer pH 6.0, loaded onto a Fractogel® SO₃-column (Millipore) and eluted with a linear gradient of 20 CV from 0-1 M NaCl. Fractions with efficiently cleaved and purified protein were identified by SDS-PAGE, pooled, concentrated and purified using a HiLoad Superdex 75 pg size exclusion column (Cytiva) with 24 HEPES pH 7, 100 mM NaCl as a running buffer. Transglutaminase containing fractions were pooled, concentrated to >20 mg/ml, flash frozen in liquid nitrogen and stored at −80° C. Enzyme activity was determined using ZediXclusive Microbial Transglutaminase assay (Zedira).

### Example 3: Exemplary ADC Preparation, Conjugation and Characterization of ADC7-M, ADC2-M, ADC5-M and ADC3-M

### 3.1. Antibody Preparation and Conjugation

Monoclonal antibodies (mAb) were stored at −80° C. Prior conjugation, mAbs were thawed at RT and buffer was exchanged to 24 mM HEPES, pH 7.0 using HiTrap Desalting columns in combination with an Äkta liquid chromatography (LC) system (Cytiva). To couple the drug linker (DL) to the antibody, a microbial transglutaminase (mTG) was used. The reaction setup was as follows: 5 mg/ml mAb, 5 molar equivalents of DL1-M per conjugation site, 20 U/ml mTG, 7% DMSO, 24 mM HEPES, pH 7.0. The reaction was carried out at 37° C. for 18 h. ADCs were separated from DL and mTG via size exclusion chromatography (SEC). Prior to SEC purification, NaCl concentration of the samples was adjusted to 100 mM using a 5 M NaCl stock solution. SEC was carried out using a HiLoad Superdex 200 26/60 Increase column in combination with an Äkta LC system (Cytiva) at a flow rate of 2.5 ml/min and 10 mM Histidine, 100 mM NaCl, pH 5.5 as running buffer. Fractionated samples containing the ADC material were pooled and concentrated to 8 mg/ml using Amicon Ultra 15 50K centrifugal (Millipore) followed by a final buffer exchange into 10 mM Histidine, 40 mM NaCl, 6% Trehalose, 0.05% TWEEN, pH 5.5 using HiTrap Desalting columns in combination with an Äkta LC system (Cytiva). Final bulk drug substance (BDS) was filtered through a 0.22 μm sterile filter unit (Millipore) and shock frozen in liquid nitrogen till further use.

### 3.2 Quality Attributes

ADC7-M

| | |
|---|---|
| Concentration[mg/ml] | 7.11 |
| Drug-to-Antibody Ratio | 3.87 |
| Purity SEC [%] | 99.9 |
| Free drug content [%] | 0.12 |
| Endotoxin content [EU/mg] | 0.34 |

ADC2-M

| | |
|---|---|
| Concentration[mg/ml] | 6.9 |
| Drug-to-Antibody Ratio | 3.86 |
| Purity SEC [%] | 99.7 |
| Free drug content [%] | 0.03 |
| Endotoxin content [EU/mg] | 0.19 |

ADC3-M

| | |
|---|---|
| Concentration[mg/ml] | 7.98 |
| Drug-to-Antibody Ratio | 4.2 |
| Purity SEC [%] | 100 |
| Free drug content [%] | 0.13 |
| Endotoxin content [EU/mg] | 0.14 |

ADC5-M

| | |
|---|---|
| Concentration[mg/ml] | 5.92 |
| Drug-to-Antibody Ratio | 3.88 |
| Purity SEC [%] | 99.8 |
| Free drug content [%] | 0.15 |
| Endotoxin content [EU/mg] | >0.5 |

### 3.3. Methods: Drug Substance Characterization

Size exclusion chromatography (SEC) method

Monomer content and purity were assessed by size exclusion chromatography on a TOSOH TSKgel column. Injection volumes ranged from 1-5 μl (up to 10 μg protein).
Wavelength 214 nm
Column Tosoh TSKgel 7.8 mm×300 mm, 5 μm
Mobile Phase 50 mM Sodium Phosphate Monobasic, 0.4 M Sodium Perchlorate, pH 6.3
Injection Volume 1-5 μL
Column Temperature 25° C.
Flow rate 0.5 mL/min

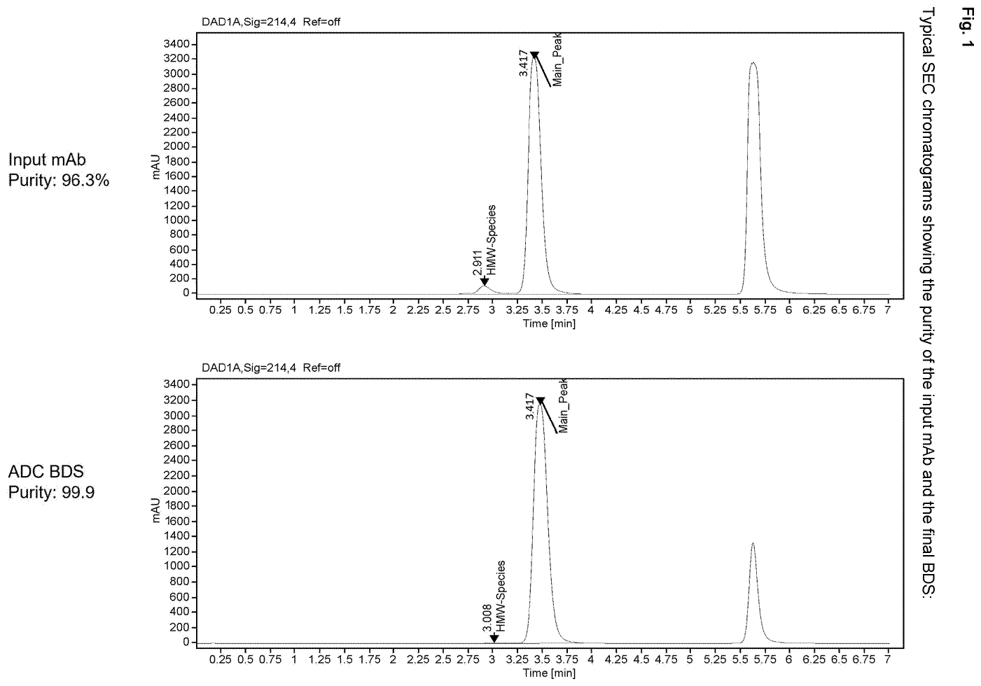

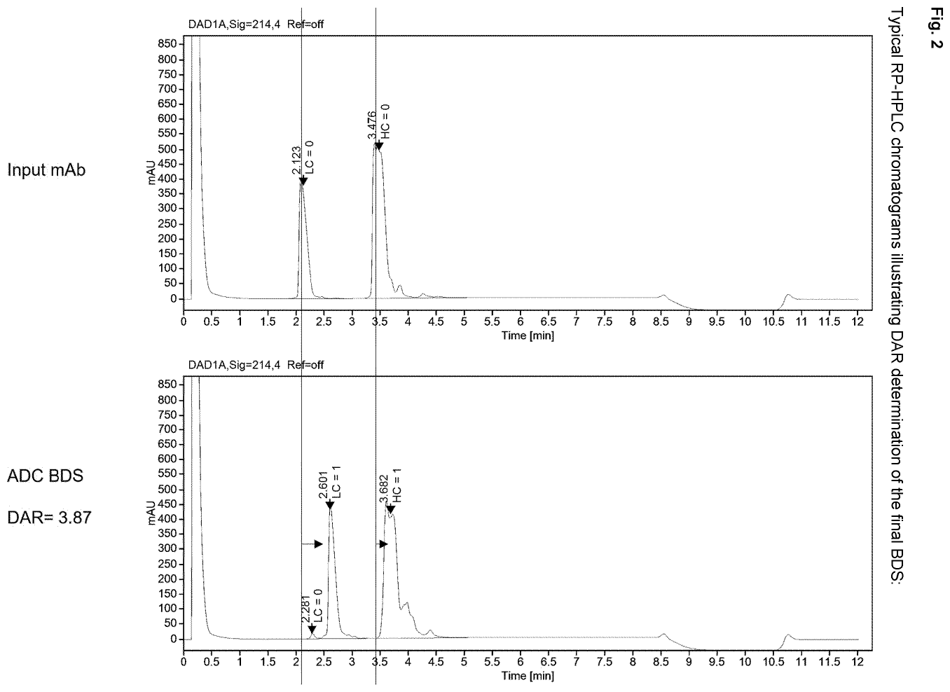

Typical SEC chromatograms showing the purity of the input mAb and the final BDS are shown in FIG. 1.

### Reversed-Phase HPLC (RP HPLC) Method

RP HPLC Method Parameters
Wavelength 214 nm
Column PLRP-S 1000 Å (50×2.1 mm, 8 μm) column, Agilent
Mobile Phase A Lichrosolv H₂O+0.1% TFA
Mobile Phase B 100% AcN+0.1% TFA
Gradient 30-45% B in 7.5 min

### Injection Volume 10 UL

Column Temperature 65° C.
Flow rate 1.0 mL/min
Sample Preparation 40 μl sample (2.2 mg/ml) were mixed with 4 μl 0.5 M TCEP and incubated for 5 min at RT. Afterwards, 40 μl of 0.5 M lodoacetamide were added and incubated for 15 min at 37° C. 10 μl of this reaction was injected.
Typical RP-HPLC chromatograms illustrating DAR determination of the final BDS are shown in FIG. 2.
Free-drug method
Wavelength 254 nm
Column Phenomenex Gemini, C18, 2×150 mm, 3 μm (P/N 00F-4439-B0)
Mobile Phase A 0.1% Formic acid in water
Mobile Phase B 0.1% Formic acid in acetonitrile

Gradient

| Time (min) | MP A (%) | MP B (%) |
|---|---|---|
| 0.00 | 95 | 5 |
| 1.00 | 95 | 5 |
| 12.00 | 0 | 100 |

3 min Equilabration to 95% A

Injection Volume 10.00 uL
Column Temperature 50° C.
Flow rate 0.75 mL/min
Sample Preparation Protein drop: 100 μL of Drug Substance+250 μL of cold MeOH+50 μL of 3 M MgCl2. Spin at 20,000 rpm for 10 min
Standard Preparation Mix 20 μL of 20 mM DL (MSC2702209A in DMSO)+20 μL DMSO+40 μL MeOH+20 μL of 200 mM NAC in Diafiltration buffer. Incubate overnight to afford 4 mM DL-NAC. Dilute 4 mM DL-NAC in MeOH to afford a 4 UM DL-NAC standard.

### Endotoxin Characterization

Endotoxin was determined by kinetic chromogenic LAL assay using an Endosafe PTS endotoxin system (Charles River). Buffers and antibodies were diluted 10-fold in LAL reagent water. The ADCs were diluted 10-fold in LAL reagent water. All samples were analyzed on 0.01-1 EU/mL cartridges. The EU/mL value was converted to EU/mg by dividing by the ADC [P] mg/mL.

### Example 4: ADC2-M Specifically Kills Cancer Cells In Vitro with High Potency

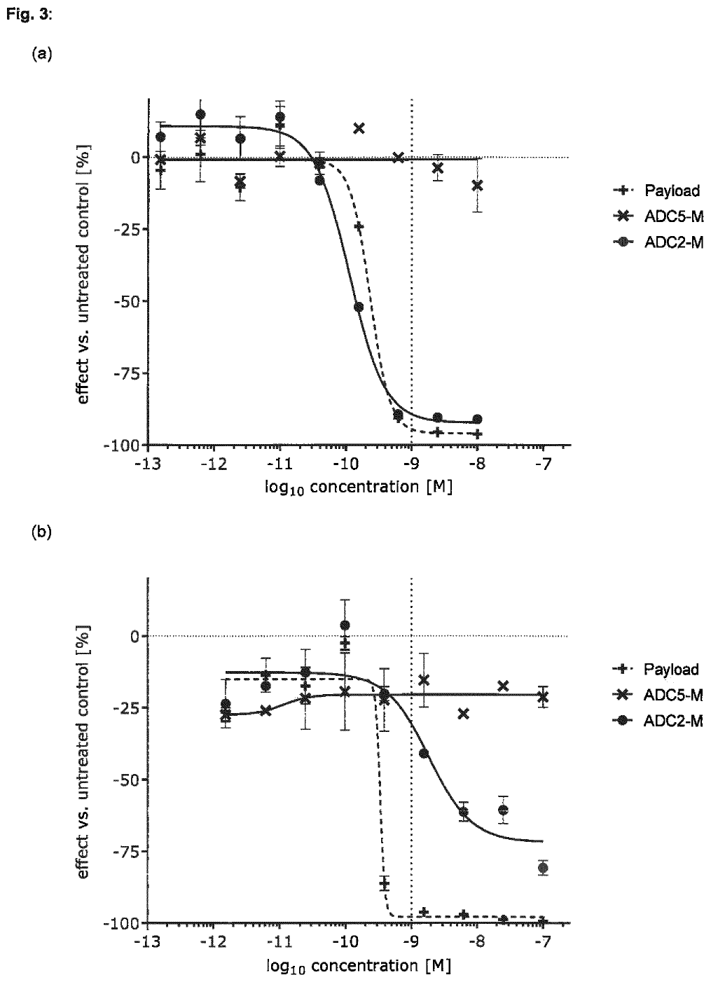

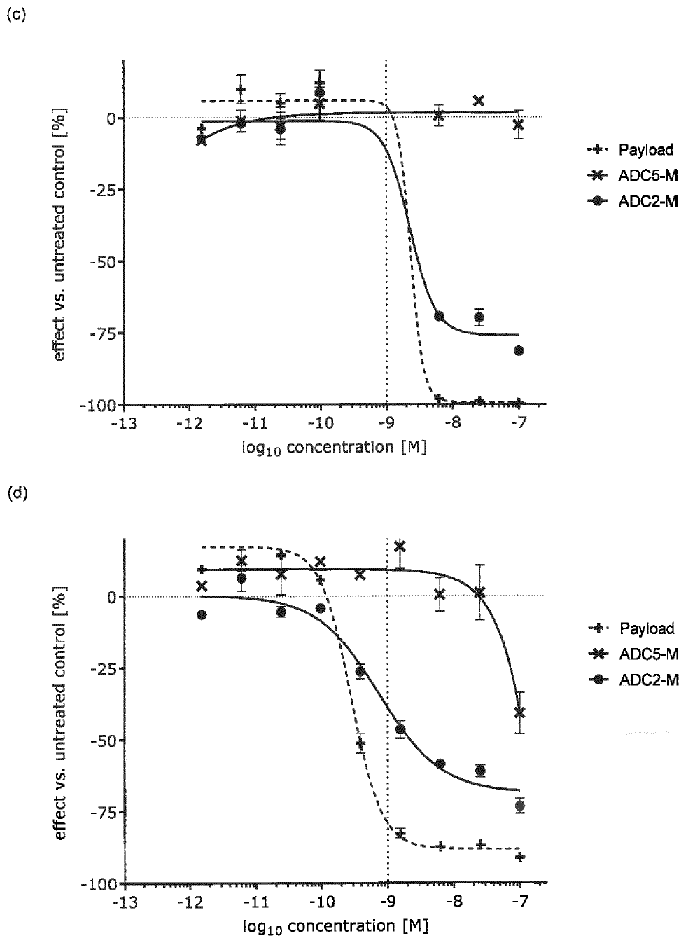

Human cancer cell lines were used to assess the potential of ADC2-M to kill cancer cells. ADC2-M showed sub-nanomolar and sub-nanomolar to single digit nanomolar in vitro potency against different CEACAM5-positive cell lines, respectively (Table 3). In contrast, effects of ADC2-M were minor on the CEACAM5-negative cell line MDA-MB-231 (Table 3). As shown in exemplary dose-response curves, ADC2-M were very potent against CEACAM5-positive cell lines SK-CO-1, SNU-16, MKN-45 and LS174T (FIG. 3a-d). In contrast, ADC2-M had only minor effects on antigen-negative MDA-MB-231 cell viability (FIG. 3e).

Isotype control ADCs utilizing the same linker payloads as ADC2-M showed much lower effects on the tested CEACAM5-positive cell lines (FIG. 3).

In conclusion, ADC2-M specifically kills CEACAM5 expressing human cancer cell lines in vitro with high potency.

TABLE 3
Potency of ADC2-M and free payload against multiple human cell lines. Maximal
effects compared to untreated controls at the highest tested compound concentration
are indicated in brackets. Cut-off for potency reporting −50% effect, otherwise
“n/a” stated. For each cell line, CEACAM5 expression is indicated.

| | SK-CO-1 | SNU-16 | MKN-45 | LS174T | MDA-MB-231 |
|---|---|---|---|---|---|
| | IC50 [M]; | IC50 [M]; | IC50 [M]; | IC50 [M]; | IC50 [M]; |
| | n ≥ 2 | n ≥ 2 | n ≥ 2 | n ≥ 2 | n ≥ 3 |
| ADC2-M | 1.1E−10 | 5.1E−9 | 1.2E−9 | 8.5E−10 | n/a |
| | (−92%) | (−78%) | (−80%) | (−67%) | (−16%) |
| Payload | 2.2E−10 | 2.9E−10 | 6.6E−10 | 3.0E−10 | 6.0E−10 |
| | (−97%) | (−99%) | (−100%) | (−91%) | (−91%) |
| CEACAM5 | Positive | Positive | Positive | Positive | Negative |

### Method-Viability Assay:

Cytotoxicity effects of the ADC on the cancer cell lines were measured by cell viability assays. Cells were seeded in a volume of 90 μL in 96-well plates the day before treatment. Test compounds (ADCs or free payloads) were formulated at 10-fold the starting concentration in cell culture medium. Test compounds were serial diluted (1:4) and 10 μL of each dilution was added to the cells in triplicates. Plates were cultured at 37° C. in a CO2 incubator for six days. For cell viability measurement, Cell Titer-Glo® reagent (Promega™ Corp, Madison, WI) was added to each well, and plates processed according to the manufacturer's instructions. Luminescence signals were measured using a Varioskan plate reader (Thermo Fisher). Luminescence readings were converted to % viability relative to untreated cells. Data was fitted with non-linear regression analysis, using log (inhibitor) vs. response, variable slope, 4-parameter fit equation using Genedata Screener or GraphPad Prism. Data is shown as % relative cell viability vs. molar compound concentration, error bars indicating standard deviation (SD) of duplicates or triplicates. Geometric mean values of IC50s derived from multiple experiments were calculated.

ADC2-M was also compared to ADC SAR DM4 in terms of its cytotoxic effect on antigen-positive SK-CO-1 and antigen-negative MDA-MB-231 cell lines. ADC2-M showed similar potency as ADC SAR DM4 against SK-CO-1 cancer cells (FIG. 3a compared to FIG. 26a). Non-specific effects against antigen-negative MDA-MB-231 were higher for ADC SAR DM4 compared to ADC2-M (FIG. 3e compared to FIG. 26b).

ADC2-M was also generated utilizing an antibody backbone lacking YTE mutation. This ADC (ADC7-M) showed comparable results like the respective ADC with YTE mutation (ADC2-M).

### Example 5: Pharmacokinetic Studies of ADCs

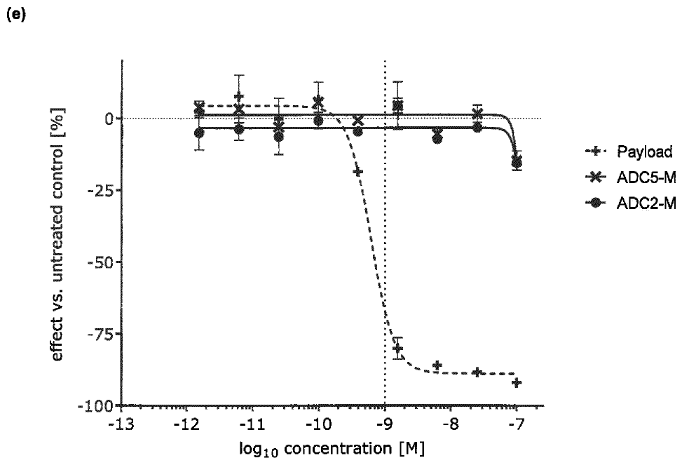

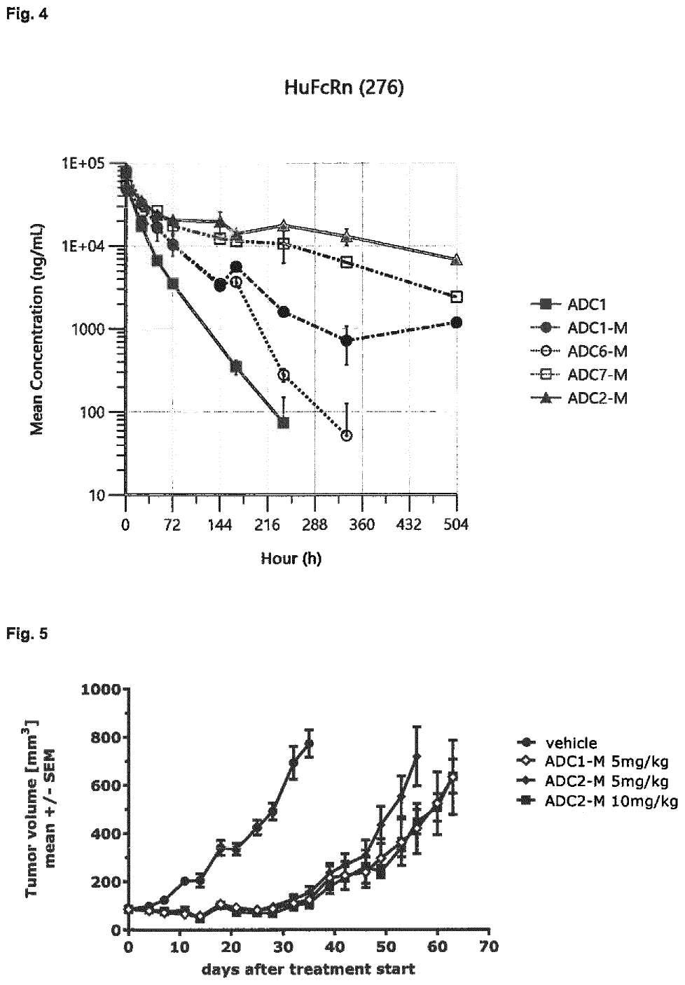

Pharmacokinetic studies in human FcRn transgenic (276 hemizygous model) mice were performed, following single intravenous administration of 3 mg/kg of ADC1, ADC1-M, ADC6-M, ADC7-M, ADC2-M. Samples were taken at 1, 24, 48, 72, 144, 168, 240, 336 and 504 h after dose, from 6 animals per treatment group for ADC1-M, ADC6-M, ADC7-M and ADC2-M, and at 0.08 (about 5 minutes), 4, 24, 48, 72, 168, 240, 336 and 504 h after dose for ADC1 from 9 animals. Data were pooled for PK analysis, see FIG. 4 for plasma concentrations per treatment. Back extrapolation of plasma concentration at time 0 (CO) resulted in slightly lower than theoretical maximal concentration for ADC1-M, ADC7-M, and ADC2-M and close to the theoretical maximum for ADC6-M and ADC1 (assuming dilution in 40 mL/kg plasma volume). The plasma concentration decreased with a biphasic profile: steep plasma concentration decrease was observed up to 24 h followed by a slower decrease up to the last sampling time (504 h) in all molecule groups except for animals treated with ADC6-M that was quantifiable up to 336 h.

The longest terminal half-life (t1/2) value was calculated for ADC2-M (190 h) a Lala-YTE-mutant with DAR-4, followed by ADC7-M (122 h) a Lala-mutant with DAR=4, ADC1-M (64.5 h) a Lala-YTE-mutant and DAR=8 and ADC6-M (33.8 h) a Lala-mutant with DAR=8 and finally ADC1 (29.6 h) an IgG1.4 with DAR=8. The % of extrapolated AUCinf was lower than 20% allowing reliable calculation of AUC0-inf and derived parameters (CI, Vz and Vss). The highest AUC0-inf and the lowest CI values were calculated for ADC2-M (Lala-YTE-mutant, DAR=4) followed by ADC7-M, ADC6-M, ADC1-M, and ADC1 in transgenic mice. The AUC0-inf and CI values ranged from 1360000 to 10200000 h*ng/ml and from 0.293 to 1.16 mL/h/kg respectively. No relevant differences in the volume of distribution (Vss) were observed with Vss ranging from 49.8 to 113 mL/kg.

In conclusion, after single iv administration of 3 mg/kg in transgenic mice the best PK profile was shown by ADC2-M with the highest plasma exposure, lowest CI and longest t1/2. PK profiles improved by moving from IgG1.4 to a Lala-mutant, further by including a YTE-mutant the strongest effect was observed by reducing DAR from 8 to 4.

TABLE 5
PK parameters for exemplary ADCs after 3 mg/kg i.v. administration. The antibody-
drug-conjugate (ADC) and drug-to-antibody ratio (DAR) is indicated as well.

| Dose | | Anti-body | | | t½ | C0 | Tlast | Clast | AUClast | AUCinf | %_Ext | Vz | CI | Vss |
|---|---|---|---|---|---|---|---|---|---|---|---|---|---|---|
| (ma/kg) | ADC | back-bone | DAR | Strain | (h) | (ng/ml) | (h) | (ng/ml) | (h * ng/ml) | (h * ng/ml) | (%) | (mL/kg) | (mL/h/kg) | (mL/kg) |
| 3 | ADC | IgG1.4 | 8 | HuFcRn | 29.6 | 76800 | 240 | 74.4 | 1360000 | 1360000 | 0.2 | 93.9 | 2.20 | 65.8 |
| | 1 | | | | | | | | | | | | | |
| 3 | ADC | Lala- | 8 | HuFcRn | 64.5 | 49900 | 504 | 1190 | 2580000 | 2590000 | 0.4 | 108 | 1.16 | 113 |
| | 1-M | YTE- | | | | | | | | | | | | |
| | | mutant | | | | | | | | | | | | |
| 3 | ADC | Lala- | 4 | HuFcRn | 190 | 56100 | 504 | 6810 | 8360000 | 10200000 | 18.4 | 80.2 | 0.293 | 87.7 |
| | 2-M | YTE- | | | | | | | | | | | | |
| | | mutant | | | | | | | | | | | | |
| 3 | ADC | Lala- | 8 | HuFcRn | 33.8 | 84100 | 336 | 52.0 | 2850000 | 2850000 | 0.1 | 51.3 | 1.05 | 49.8 |
| | 6-M | Mutant | | | | | | | | | | | | |
| 3 | ADC | Lala- | 4 | HuFcRn | 122 | 55800 | 504 | 2400 | 5720000 | 6140000 | 6.9 | 86.0 | 0.488 | 93.4 |
| | 7-M | Mutant | | | | | | | | | | | | |

### Example 6: Efficacy of ADC1-M and ADC2-M in a Pancreatic Cell Line Derived Tumor Model

Efficacy of ADC1-M and ADC2-M has been evaluated in the human pancreatic, cell line derived xenograft model BxPC3 (ATCC, CRL-1687). 5×10⁶ BxPC3 cells were injected subcutaneously into the right flank of six to eight weeks old immunodeficient female mice (Hsd: Athymic Nude-Foxn1nu, Envigo). When tumors reached a mean volume of 85 mm³, 10 mice/group were treated once intravenously with vehicle (saline solution) or ADC1-M (5 mg/kg; day 0) or with ADC2-M (5 mg/kg or 10 mg/kg; day 0). Tumor length (L) and width (W) were measured with calipers and tumor volumes were calculated using L×(W\hat{ }2)/2.

The single treatment with ADC1-M at a dose of 5 mg/kg and with ADC2-M at a dose of 5 mg/kg or 10 mg/kg led to a significant anti-tumor effect. Both ADCs have comparable efficacy in this model (FIG. 5). The treatments had no significant effect on body weight (data not shown).

### Example 7: Preparation of ADC8, an Analog of the ADC Labetuzumab Govitecan

7.1 The antibody

For the purposes of further comparative experiments, a further analog of the ADC labetuzumab govitecan was prepared based on a monoclonal antibody having the following sequence:

Heavy chain:
(SEQ ID NO: 62)
DIQLTQSPSS LSASVGDRVT ITCKASQDVG TSVAWYQQKP GKAPKLLIYW
TSTRHTGVPS RFSGSGSGTD FTFTISSLQP EDIATYYCQQ YSLYRSFGQG TKVEIKRTVA
APSVFIFPPS DEQLKSGTAS VVCLLNNFYP REAKVQWKVD NALQSGNSQE
SVTEQDSKDS TYSLSSTLTL SKADYEKHKV YACEVTHQGL SSPVTKSFNR GECEVQLVES
GGGVVQPGRS LRLSCSASGF DFTTYWMSWV RQAPGKGLEW IGEIHPDSST
INYAPSLKDR FTISRDNAKN TLFLQMDSLR PEDTGVYFCA SLYFGFPWFA YWGQGTPVTV
SSASTKGPSV FPLAPSSKST SGGTAALGCL VKDYFPEPVT VSWNSGALTS
GVHTFPAVLQ SSGLYSLSSV VTVPSSSLGT QTYICNVNHK PSNTKVDKRV EPKSCDKTHT
CPPCPAPELL GGPSVFLFPP KPKDTLMISR TPEVTCVVVD VSHEDPEVKF
NWYVDGVEVH NAKTKPREEQ YNSTYRVVSV LTVLHQDWLN GKEYKCKVSN
KALPAPIEKT ISKAKGQPRE PQVYTLPPSR EEMTKNQVSL TCLVKGFYPS DIAVEWESNG
QPENNYKTTP PVLDSDGSFF LYSKLTVDKS RWQQGNVFSC SVMHEALHNH
YTQKSLSLSP GK
Light chain:
(SEQ ID NO: 63)
DIQLTQSPSS LSASVGDRVT ITCKASQDVG TSVAWYQQKP GKAPKLLIYW
TSTRHTGVPS RFSGSGSGTD FTFTISSLQP EDIATYYCQQ YSLYRSFGQG TKVEIKRTVA
APSVFIFPPS DEQLKSGTAS VVCLLNNFYP REAKVQWKVD NALQSGNSQE
SVTEQDSKDS TYSLSSTLTL SKADYEKHKV YACEVTHQGL SSPVTKSFNR GEC

### 7.2 the Drug-Linker Compound

As a drug-linker molecule to be conjugated to the above-mentioned antibody, a molecule of the following structure was used:

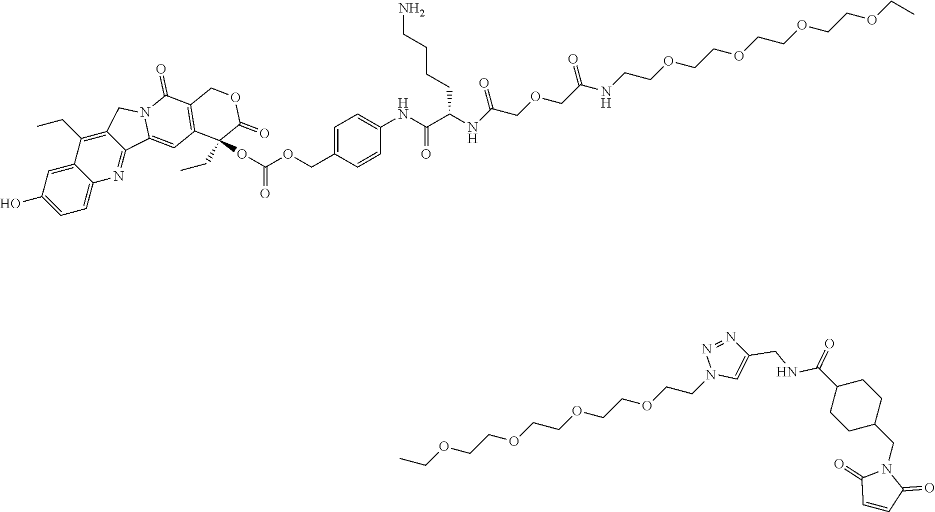

This drug-linker molecule was purchased from SyntaBio LLC, 10239 Flanders Ct, San Diego, C_{A} 92121. Lot No. S041070422.

### 7.3 Conjugation

The monoclonal antibody (mAb) was thawed at 2-8° C. up to 3 days prior to conjugation and stored at 2-8° C. in PBS pH 6.8 until further use. On the day of conjugation, the pH of the mAb solution was adjusted by addition of 0.5 M Tris, 0.025 M EDTA, pH 8.5 to a final concentration of 5% (v/v). After pH adjustment, the mAb was reduced using 10 molar equivalents of TCEP and an incubation at 20° C. for 120 min. Subsequently, the mAb solution was diluted 1:1 with 20 mM Histidine, 80 mM NaCl, pH 5.5, the DMSO concentration was adjusted to 10% (v/v) and 16 molar equivalents of the above-mentioned drug-linker were added to start the reaction. The reaction was incubated at 20° C. for 60 min and was finally quenched by addition of 100 mM NAC (n-acetyl-cysteine). Afterwards, the conjugated mAb (i.e. the ADC) was processed via preparative size exclusion chromatography.

### 7.4 Preparative Size Exclusion Chromatography, Desalting and Filtration

The reaction mixture was purified using preparative size-exclusion chromatography. A GE HiLoad 26/60 Superdex S200 column was connected to an Äkta Avant 25 system (GE Healthcare) and equilibrated with 20 mM Histidine, 80 mM NaCl, pH 5.5 according to the manufacturers' instructions. Subsequently, the reaction mixture was injected and run through the column with a flowrate of 5 ml/min using 20 mM Histidine, 80 mM NaCl, pH 5.5 as running buffer. ADC-containing fractions were determined via UV light absorption at 280 nm, pooled and concentrated. ADC material was concentrated using Vivaspin VS2022 devices (Sartorius UK Ltd.) according to manufacturer's instructions. The concentrated ADC material was transferred into formulation buffer (10 mM Histidine 100 mM NaCl, 3% trehalose, 0.05% (w/v) PS20, pH 5.5) using HiPrep 26/10 desalting columns (GE Healthcare) at a flowrate of 10 ml/min on an Äkta Avant 25 system (GE Healthcare) according to the manufacturer's instructions. The final ADC material was filtered using a 0.2 μm filter (0.2 μm PES filters, Merck Millipore), aliquoted and subsequently shock frozen in liquid nitrogen. Final concentration of the ADC material (drug substance) was 7.7 mg/ml. The material was kept at −80° C. until further use. The ADC resulting from this work is referred to herein as “ADC8”; this ADC is an analog of labetuzumab govitecan.

### 7.5 Further Characterization of ADC8 Drug Substance

The ADC8 drug substance obtained above was further analyzed by (a) size exclusion chromatography (SEC), showing a monomeric purity of 99.3%, (b) reversed-phase HPLC (RP HPLC), showing a DAR of 7.7, and (c) an RP HPLC-based free-drug method, showing residual free-drug levels below 0.02% (by molar ratio).

### Example 8: ADC1-M, ADC2-M, ADC6-M and ADC7-M Kill Cancer Cells with Higher Specificity than ADC SAR DM4 (an Analog of Tusamitamab Ravtansine) and ADC8 (an Analog of Labetuzumab Govitecan)

Using the same method as described in Example 21, human cancer cell lines were used to compare effects of ADC1-M, ADC2-M, ADC6-M and ADC7-M, as well as ADC SAR DM4 and ADC8, on cancer cells with CEACAM5 expression relative to effects on cancer cells lacking CEACAM5 expression. A fold reduction in IC50, defined as a SPECIFICITY FACTOR, was calculated by dividing the IC50 against CEACAM5-negative MDA-MB-231 cells by the IC50 against each CEACAM5-positive cell line (see Table 6). The larger the value of the SPECIFICITY FACTOR is, the more specific is the tested ADC. ADC1-M, ADC2-M, ADC6-M and ADC7-M showed much lower IC50s in the CEACAM5-positive SK-CO-1, SNU-16, and MKN-45 cells than in the CEACAM5-negative MDA-MB-231 cells, which resulted in SPECIFICITY FACTORS in the range of 116 to 874. SPECIFICITY FACTORS for ADC2-M and ADC7-M are likely underestimated due to the lack of effect on MDA-MB-231 cells in the tested concentration range (as shown for ADC2-M in Table 3 and FIG. 3e). For this reason, for ADC2-M and ADC7-M only, the IC50 was set to the highest tested concentration of 100 nM to calculate SPECIFICITY FACTORS. In contrast, ADC SAR DM4 and ADC8 showed lower SPECIFICITY FACTORS in the range of 26 to 73 and 1.8 to 4.8, respectively. In conclusion, a more selective killing of CEACAM5-positive cancer cells by ADC1-M, ADC2-M, ADC6-M and ADC7-M compared with ADC SAR DM4 and ADC8 in vitro was shown.

TABLE 6
Relative potency increases of ADCs in CEACAM5-positive cell
lines compared to CEACAM5-negative MDA-MB-231 cell line
(SPECIFICITY FACTOR). IC50 in MDA-MB-231 was divided by
IC50 in each of the three CEACAM5-positive cell lines to
calculate the respective SPECIFICITY FACTOR for each ADC
(the theoretical factor of 1 for MDA-MB-231 being reported
only for illustration). Calculations were based on IC50
geometric means of 2 to 4 individual experiments.

| | SK-CO-1 | SNU-16 | MKN-45 | MDA-MB-231 |
|---|---|---|---|---|
| | n ≥ 2 | n ≥ 2 | n ≥ 2 | n ≥ 2 |
| ADC1-M | 874.4 | 241.9 | 201.2 | 1 |
| ADC2-M | >631.9 | >207.2 | >166.6 | 1 |
| ADC6-M | 710.2 | 194.9 | 157.7 | 1 |
| ADC7-M | >595.8 | >178.9 | >116.2 | 1 |
| ADC SAR DM4 | 46.5 | 73.2 | 26.0 | 1 |
| ADC8 | 4.8 | 1.8 | 2.5 | 1 |
| CEACAM5 | Positive | Positive | Positive | Negative |

### Example 9: ADC1-M, ADC2-M, ADC6-M, ADC7-M Mediate a More Potent Bystander Effect than ADC SAR DM4 Against Antigen-Negative Cells in Co-Culture with Antigen-Positive Cells

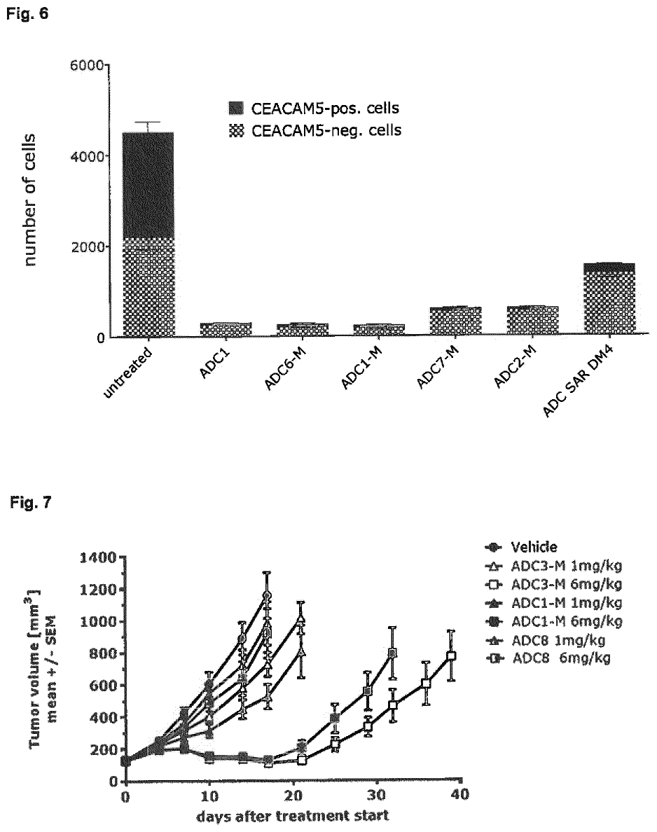

Using the same method as in Example 10, the potential of ADC1-M, ADC2-M, ADC6-M, ADC7-M and ADC SAR DM4 to mediate a bystander effect against antigen-negative cells in close proximity to antigen-positive cells was evaluated in bystander assays. ADC1-M, ADC2-M, ADC6-M and ADC7-M showed a potent bystander effect against CEACAM5-negative MDA-MB-231 cells in the presence of CEACAM5-positive SK-CO-1 (FIG. 6). The co-culture experiments were performed at an ADC concentration of 1 nM which, for ADC1, causes maximal inhibition of CEACAM5-positive SK-CO-1 cell viability (FIG. 26A) but no effect on CEACAM5-negative MDA-MB-231 cells alone (FIG. 26B). In line with these findings, no non-specific effect of ADC1-M, ADC2-M, ADC6-M, ADC7-M and ADC SAR DM4 was observed in the bystander assay setup for MDA-MB-231 only controls.

In conclusion, ADC1-M, ADC2-M, ADC6-M, ADC7-M mediate a potent bystander effect against antigen-negative cancer cells in co-culture with antigen-positive cells. These findings indicate the potential to effectively target tumors with heterogeneous target expression.

Compared to ADC SAR DM4, ADC1-M, ADC2-M, ADC6-M and ADC7-M mediated a much more potent bystander effect on antigen-negative cells in co-culture with antigen-positive cells (FIG. 6).

For all ADCs tested, the extent of bystander effect increased with increasing the number of antigen-positive cells added to a constant number of antigen-negative cells. Without wishing to be bound by theory, this may be a result of more ADC being processed by the higher number of antigen-positive cells to release more free payload, which is responsible for the bystander effect on antigen-negative cells.

No non-specific effects of tested ADCs were observed on MDA-MB-231 cells alone for all tested ADCs at 1E-9M testing concentration in bystander assays.

### Example 10: Efficacy of ADC1-M and ADC3-M Compared to ADC8

Efficacy of ADC1-M and ADC3-M in comparison to ADC8 was evaluated in the human pancreatic adenocarcinoma cell line-derived xenograft model HPAF-II (ATCC, CRL-1997). Six to eight weeks old immunodeficient female mice (Hsd: Athymic Nude-Foxn1nu, Envigo) were injected subcutaneously in the right flank with 5×106 HPAF-II cells. When tumors reached a mean volume of 150 mm³, 10 mice/group were treated once intravenously with ADC1-M (1 mg/kg or 6 mg/kg) or with ADC3-M (1 mg/kg or 6 mg/kg) or with ADC8 (1 mg/kg or 6 mg/kg). Tumor length (L) and width (W) were measured with calipers and tumor volumes were calculated using the formula L×W\hat{ }2/2.

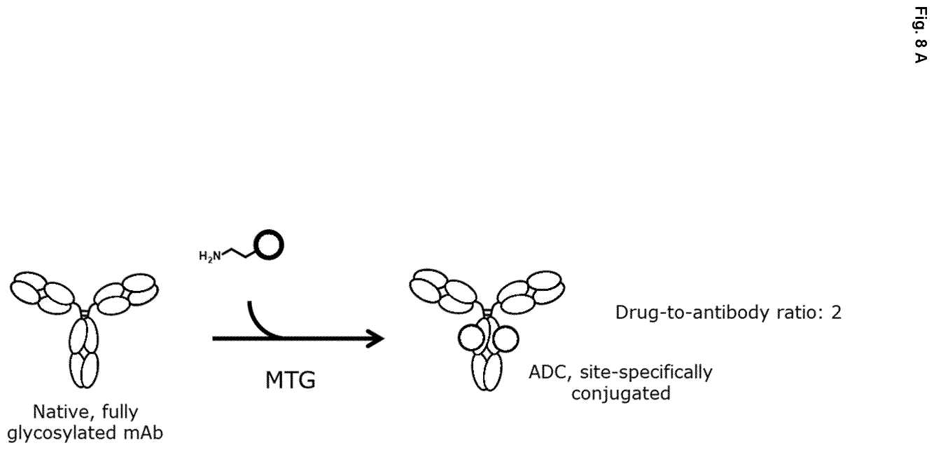

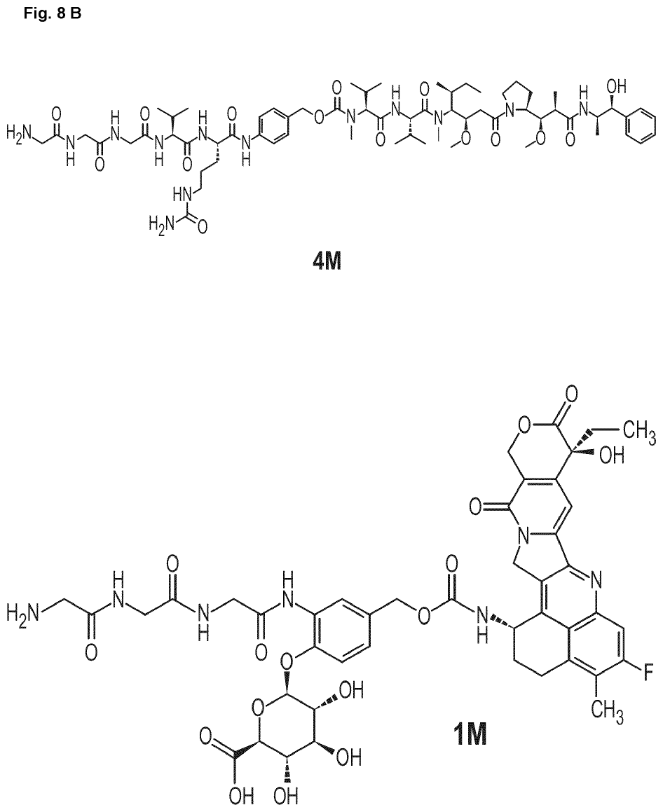

The single treatment with ADC1-M or ADC3-M at a dose of 1 or 6 mg/kg led to a significant anti-tumor effect. The effect is dose-dependent, as the single treatment with 1 mg/kg only led to a mild and temporary anti-tumor effect, while the higher dosage shows a much stronger anti-tumor effect. In contrast, the single treatment with same doses of ADC8 showed no significant anti-tumor effect at either dose (FIG. 7). All treatments had no significant effect on body weight (data not shown).

### Example 11: Development of Improved Conjugation Reaction Conditions

### 11.1 mTG Variants

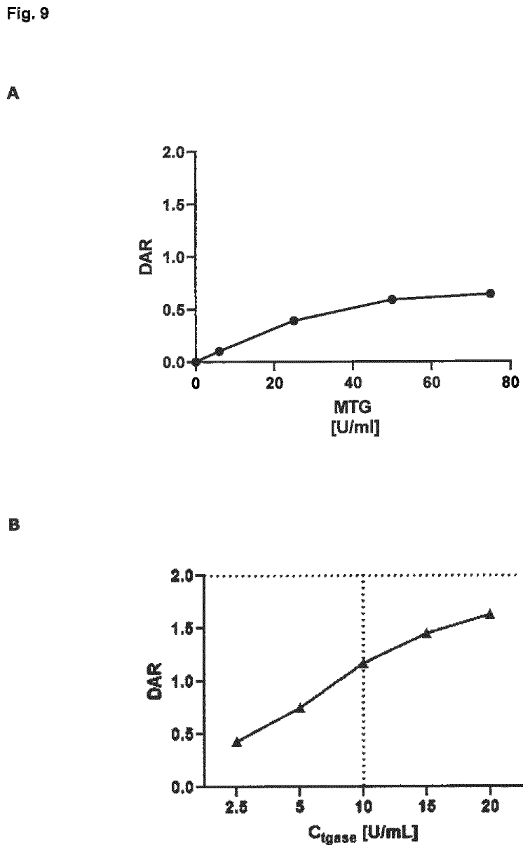

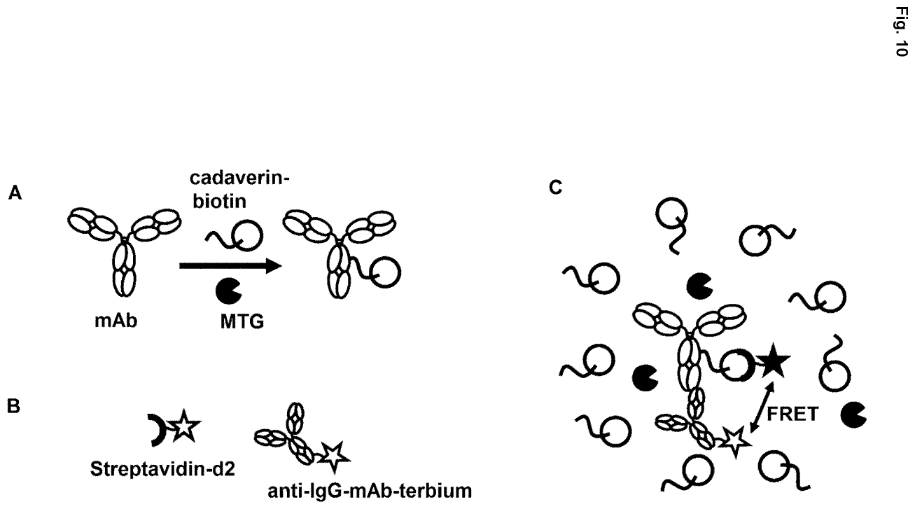

Site-specific conjugation to position Q295 (see also: figure legend of FIG. 9) of native mAbs via commonly used mTG conjugation technology is not very efficient and does not allow generation of homogenous ADCs with a DAR=2 (see FIG. 9A).

Even when using alternative mTG variants such as from S. ladakanum, the results using prior art buffer systems were ineffective (see FIG. 9B).

### 11.2 Buffer Composition, Excipient and pH

As mentioned, although mTG from S. ladakanum shows a better conjugation efficiency to position Q295 compared to mTG from S. mobaraensis, conjugation efficiency using published reaction conditions is still not optimal. Therefore, a high throughput (HT) assay was developed that is able to efficiently screen for improved reaction conditions regarding, e.g. buffer composition, excipients, pH, etc. This assay (FIG. 10) consists of a first conjugation step, in which a biotin-comprising, mTG-compatible substrate is conjugated using mTG under varying reaction conditions. Efficacy of conjugation is afterwards determined via a homogeneous time-resolved fluorescence (HTRF) assay, which allows conducting the assay with high-throughput.

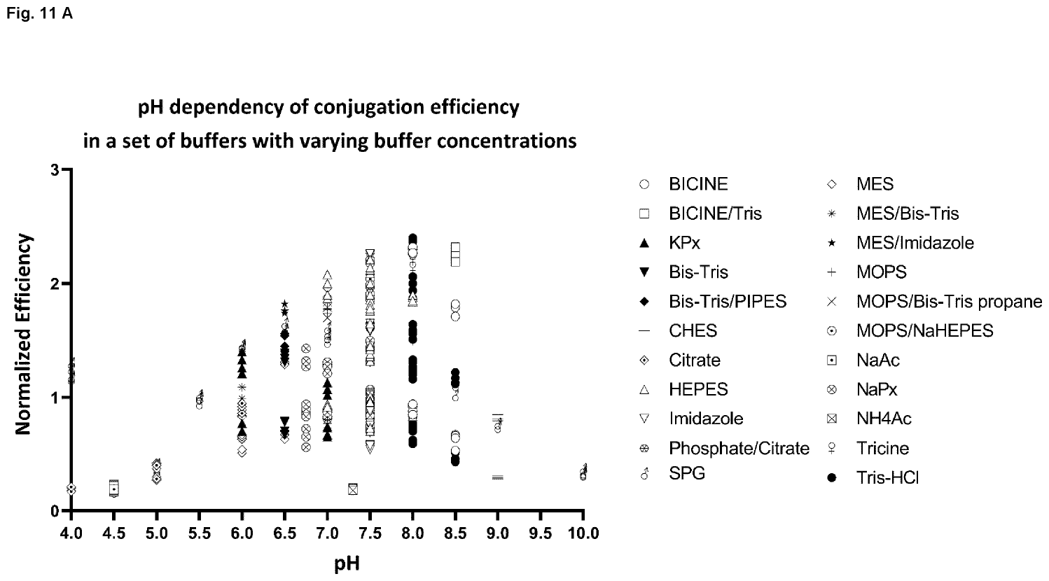

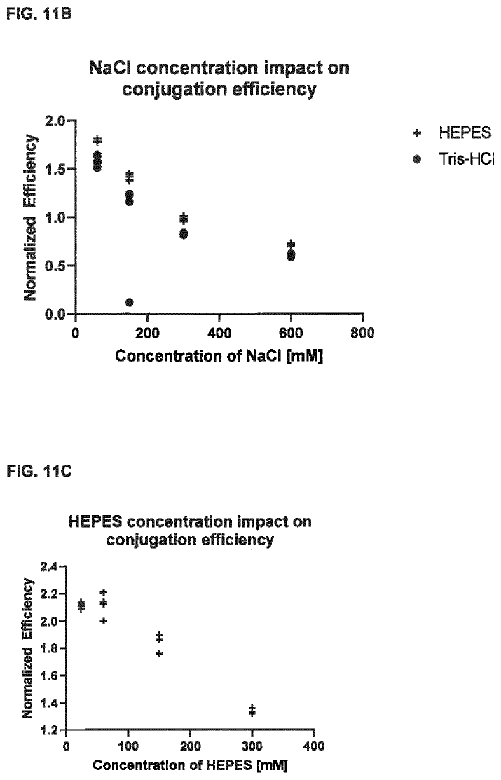

More than 10000 different reaction conditions were screened and reaction parameters were identified which increased conjugation efficiency. Procedures for conjugation reaction and assessment of conjugation efficiency are described in sections 11.3.1 to 11.3.3. FIGS. 11 (A), (B) and (C) show the impact of pH (A), salt (B) and buffer substance concentration (C) on mTG conjugation efficiency.

It was found that a pH of about 5.9-8.7 was beneficial in the conjugation reaction (FIG. 11 (A)). Furthermore, it was found that reducing the sodium chloride concentration improved the conjugation efficiency (FIG. 11 (B)). Regarding the concentration of the buffering substance, exemplarily shown for experiments using HEPES, it was found that lower concentrations of buffering agent were also beneficial.

The finding that the most suitable reaction buffers were those containing no sodium chloride and low concentrations of buffering agents was surprising, since physiological concentrations of salt are typically used in such reactions.

In terms of normalized conjugation efficiency, it was unexpectedly shown also that the type of buffering agent and the pH played a role as shown in the table below (excerpt from FIG. 11A):

| C1 | Compo- | C2 | Compo- | | normalized |
|---|---|---|---|---|---|
| [mM] | nent 1 | [mM] | nent 2 | pH | efficiency |
| 120 | Citrate | 0 | | 4 | 0.17 |
| 120 | Citrate | 0 | | 4 | 0.17 |
| 120 | NaAc | 300 | NaCl | 4.5 | 0.17 |
| 120 | SPG | 0 | | 4.5 | 0.17 |
| 120 | SPG | 0 | | 4.5 | 0.17 |
| 120 | Citrate | 0 | | 4 | 0.18 |
| 120 | NaAc | 0 | | 4.5 | 0.18 |
| 120 | Citrate | 300 | NaCl | 4 | 0.18 |
| 0 | | 0 | | NA | 0.18 |
| 120 | Citrate | 300 | NaCl | 4 | 0.18 |
| 120 | Citrate | 300 | NaCl | 4 | 0.18 |
| 120 | NaAc | 300 | NaCl | 4.5 | 0.18 |
| 120 | NH4Ac | 300 | NaCl | 7.3 | 0.18 |
| 0 | | 0 | | NA | 0.18 |
| 120 | NH4Ac | 300 | NaCl | 7.3 | 0.18 |
| 120 | NH4Ac | 300 | NaCl | 7.3 | 0.18 |
| 120 | HEPES | 0 | | 7 | 2 |
| 60 | Tris-HCl | 0 | | 8 | 2 |
| 60 | HEPES | 0 | | 7.5 | 2 |
| 60 | MOPS/NaHEPES | 0 | | 7.5 | 2.04 |
| 60 | Tris-HCl | 0 | | 8 | 2.06 |
| 120 | HEPES | 0 | | 7 | 2.08 |
| 24 | HEPES | 0 | | 7.5 | 2.09 |
| 24 | HEPES | 0 | | 7.5 | 2.11 |
| 24 | HEPES | 0 | | 7.5 | 2.12 |
| 60 | HEPES | 0 | | 7.5 | 2.12 |
| 120 | Tricine | 0 | | 8 | 2.14 |
| 24 | HEPES | 0 | | 7.5 | 2.14 |
| 60 | HEPES | 0 | | 7.5 | 2.14 |
| 60 | MOPS/NaHEPES | 0 | | 7.5 | 2.18 |
| 60 | BICINE/Tris | 0 | | 8.5 | 2.19 |
| 120 | Tricine | 0 | | 8 | 2.21 |
| 60 | HEPES | 0 | | 7.5 | 2.21 |
| 60 | MOPS/NaHEPES | 0 | | 7.5 | 2.21 |
| 120 | Imidazole | 0 | | 7.5 | 2.23 |
| 120 | Tricine | 0 | | 8 | 2.24 |
| 60 | BICINE/Tris | 0 | | 8.5 | 2.24 |
| 60 | BICINE/Tris | 0 | | 8.5 | 2.24 |
| 120 | Imidazole | 0 | | 7.5 | 2.25 |
| 120 | Imidazole | 0 | | 7.5 | 2.26 |
| 120 | BICINE | 0 | | 8 | 2.26 |
| 120 | Imidazole | 0 | | 7.5 | 2.26 |
| 24 | Tris-HCl | 0 | | 8 | 2.26 |
| 120 | BICINE | 0 | | 8 | 2.27 |
| 120 | Tricine | 0 | | 8 | 2.28 |
| 120 | BICINE | 0 | | 8 | 2.3 |
| 24 | Tris-HCl | 0 | | 8 | 2.31 |
| 120 | BICINE | 0 | | 8 | 2.32 |
| 60 | BICINE/Tris | 0 | | 8.5 | 2.32 |
| 24 | Tris-HCl | 0 | | 8 | 2.37 |
| 24 | Tris-HCl | 0 | | 8 | 2.4 |

As shown above, a too acidic pH or too much sodium chloride resulted in poorer conjugation efficiency. Furthermore, the following types of buffering agents at a pH of between 7 and 8.5 resulted in a high conjugation efficiency: HEPES, Tris-HCl, MOPS/NaHEPES, BICINE, BICINE/Tris, Imidazole and Tricine.

Conjugation of drug-linker 4M was then evaluated using the suitable buffers selected from the high-throughput screening results. Procedures for conjugation reaction and assessment of conjugation efficiency are described in sections 11.3.4 and 11.3.5. The results confirm highly improved conjugation efficiency in all selected buffers as compared to previously used buffer (see FIGS. 12A and 12B). Two very good performing buffers included a buffer comprising 24 mM HEPES at a pH of 7.5 (see FIG. 12A) and a buffer comprising 24 mM Tris-HCl at pH 8. These buffers provided much higher DARs than a reaction in a prior art buffer (25 mM Tris-HCl, 150 mM NaCl, pH 8.0).

With the improved reaction conditions, reduced amounts of the mTG enzyme were necessary to effectively conjugate the linker to the antibody compared to when the reaction was carried out in prior art buffer systems.

### 11.3 Method Summary

A Method for Expression and Purification of Transglutaminase has been Described Above in Example 2.

### 11.3.1 Conjugation Reaction Method for High Throughput Bioconjugation Screening

2 μL 5× concentrated buffer plates (displayed in Table 7) (Molecular Dimensions) were mixed with 6 μL of pre-diluted additives (displayed in Table 8) (Jena Bioscience). Herceptin (Trastuzumab, Genentech/Roche) and N-(biotinyl) cadaverine (B002, Zedira) were premixed and 1 μL of the mix was added to the buffer dilutions yielding 5 mg/mL herceptin and 687.6 μM N-(biotinyl) cadaverine final in the reaction mixture. The enzyme “MTG_1” (mTG enzyme according to SEQ ID NO: 45) was added in 1 μL to yield 12.5 U/mL final. The transglutaminase inhibitor C102 (Zedira) was added to 0.1 mM final after 16 hours of incubation at 37° C. to stop the reaction. The transglutaminase reaction was transferred to a high throughput screening system and evaluated with the HTRF readout.

Controls for the screening: Inhibitor was added prior to MTG_1 addition to one well per plate as a negative control to measure the background during HTRF readout.

TABLE 7
Customized buffers used for screening of reaction conditions. All buffers were 5X
concentrated:

| Well | | | | | | | |
|---|---|---|---|---|---|---|---|
| No. | Conc. | Units | Reagent | Conc | Units | Buffer | pH |
| A1 | 0.5 | M | Sodium | 1.25 | M | Sodium phosphate | 6.75 |
| | | | chloride | | | monobasic | |
| A2 | | | | 0.595 | M | Citrate | 4.0 |
| A3 | | | | 0.595 | M | Sodium acetate | 4.5 |
| A4 | | | | 0.595 | M | Citrate | 5.0 |
| A5 | | | | 0.595 | M | MES | 6.0 |
| A6 | | | | 0.595 | M | Potassium phosphate | 6.0 |
| A7 | | | | 0.595 | M | Citrate | 6.0 |
| A8 | | | | 0.595 | M | Bis-Tris | 6.5 |
| A9 | | | | 0.595 | M | MES | 6.5 |
| A10 | | | | 0.595 | M | Sodium phosphate | 7.0 |
| | | | | | | monobasic | |
| A11 | | | | 0.595 | M | Potassium phosphate | 7.0 |
| | | | | | | monobasic | |
| A12 | | | | 0.595 | M | HEPES | 7.0 |
| B1 | 0.75 | M | Sodium | 0.125 | M | Tris-HCl | 8 |
| | | | chloride | | | | |
| B2 | | | | 0.595 | M | Ammonium acetate | 7.3 |
| B3 | | | | 0.595 | M | Tris-HCl | 7.5 |
| B4 | | | | 0.595 | M | Sodium phosphate | 7.5 |
| | | | | | | monobasic | |
| B5 | | | | 0.595 | M | Imidazole | 7.5 |
| B6 | | | | 0.595 | M | HEPES | 8.0 |
| B7 | | | | 0.595 | M | Tris-HCl | 8.0 |
| B8 | | | | 0.595 | M | Tricine | 8.0 |
| B9 | | | | 0.595 | M | BICINE | 8.0 |
| B10 | | | | 0.595 | M | BICINE | 8.5 |
| B11 | | | | 0.595 | M | Tris-HCl | 8.5 |
| B12 | | | | 0.595 | M | CHES | 9.0 |
| C1 | | | | 0.125 | M | Tris-HCl | 8 |
| C2 | | | | 0.595 | M | Citrate | 4.0 |
| C3 | | | | 0.595 | M | Sodium acetate | 4.5 |
| C4 | | | | 0.595 | M | Citrate | 5.0 |
| C5 | | | | 0.595 | M | MES | 6.0 |
| C6 | | | | 0.595 | M | Potassium phosphate | 6.0 |
| | | | | | | monobasic | |
| C7 | | | | 0.595 | M | Citrate | 6.0 |
| C8 | 1.49 | M | Sodium | 0.595 | M | Bis-Tris | 6.5 |
| | | | chloride | | | | |
| C9 | 1.49 | M | Sodium | 0.595 | M | MES | 6.5 |
| | | | chloride | | | | |
| C10 | 1.49 | M | Sodium | 0.595 | M | Sodium phosphate | 7.0 |
| | | | chloride | | | monobasic | |
| C11 | 1.49 | M | Sodium | 0.595 | M | Potassium phosphate | 7.0 |
| | | | chloride | | | monobasic | |
| C12 | 1.49 | M | Sodium | 0.595 | M | HEPES | 7.0 |
| | | | chloride | | | | |
| D1 | 0.75 | M | Sodium | 0.125 | M | Tris-HCl | 8 |
| | | | chloride | | | | |
| D2 | 1.49 | M | Sodium | 0.595 | M | Ammonium acetate | 7.3 |
| | | | chloride | | | | |
| D3 | 1.49 | M | Sodium | 0.595 | M | Tris-HCl | 7.5 |
| | | | chloride | | | | |
| D4 | 1.49 | M | Sodium | 0.595 | M | Sodium phosphate | 7.5 |
| | | | chloride | | | monobasic | |
| D5 | 1.49 | M | Sodium | 0.595 | M | Imidazole | 7.5 |
| | | | chloride | | | | |
| D6 | 1.49 | M | Sodium | 0.595 | M | HEPES | 8.0 |
| | | | chloride | | | | |
| D7 | 1.49 | M | Sodium | 0.595 | M | Tris-HCl | 8.0 |
| | | | chloride | | | | |
| D8 | 1.49 | M | Sodium | 0.595 | M | Tricine | 8.0 |
| | | | chloride | | | | |
| D9 | 1.49 | M | Sodium | 0.595 | M | BICINE | 8.0 |
| | | | chloride | | | | |
| D10 | 1.49 | M | Sodium | 0.595 | M | BICINE | 8.5 |
| | | | chloride | | | | |
| D11 | 1.49 | M | Sodium | 0.595 | M | Tris-HCl | 8.5 |
| | | | chloride | | | | |
| D12 | 1.49 | M | Sodium | 0.595 | M | CHES | 9.0 |
| | | | chloride | | | | |
| E1 | 0.75 | M | Sodium | 0.125 | M | Tris-HCl | 8 |
| | | | chloride | | | | |
| E2 | | | | 0.595 | M | SPG | 4.5 |
| E3 | | | | 0.595 | M | SPG | 5.0 |
| E4 | | | | 0.595 | M | SPG | 5.5 |
| E5 | | | | 0.595 | M | SPG | 6.0 |
| E6 | | | | 0.595 | M | SPG | 6.5 |
| E7 | | | | 0.595 | M | SPG | 7.0 |
| E8 | | | | 0.595 | M | SPG | 7.5 |
| E9 | | | | 0.595 | M | SPG | 8.0 |
| E10 | | | | 0.595 | M | SPG | 8.5 |
| E11 | | | | 0.595 | M | SPG | 9.0 |
| E12 | | | | 0.595 | M | SPG | 10.0 |
| F1 | | | | 0.12 | M | HEPES | 7.5 |
| F2 | | | | 0.3 | M | HEPES | 7.5 |
| F3 | | | | 0.745 | M | HEPES | 7.5 |
| F4 | | | | 1.49 | M | HEPES | 7.5 |
| F5 | | | | 0.12 | M | Sodium phosphate | 7.5 |
| | | | | | | monobasic | |
| F6 | | | | 0.3 | M | Sodium phosphate | 7.5 |
| | | | | | | monobasic | |
| F7 | | | | 0.745 | M | Sodium phosphate | 7.5 |
| | | | | | | monobasic | |
| F8 | | | | 1.49 | M | Sodium phosphate | 7.5 |
| | | | | | | monobasic | |
| F9 | | | | 0.12 | M | Tris-HCl | 8.0 |
| F10 | | | | 0.3 | M | Tris-HCl | 8.0 |
| F11 | | | | 0.745 | M | Tris-HCl | 8.0 |
| F12 | | | | 1.49 | M | Tris-HCl | 8.0 |
| G1 | 0.3 | M | Sodium | 0.3 | M | HEPES | 7.5 |
| | | | chloride | | | | |
| G2 | 0.745 | M | Sodium | 0.3 | M | HEPES | 7.5 |
| | | | chloride | | | | |
| G3 | 1.49 | M | Sodium | 0.3 | M | HEPES | 7.5 |
| | | | chloride | | | | |
| G4 | 2.975 | M | Sodium | 0.3 | M | HEPES | 7.5 |
| | | | chloride | | | | |
| G5 | | | | 0.595 | M | MOPS | 7.0 |
| G6 | 1.49 | M | Sodium | 0 | | | |
| | | | chloride | | | | |
| G7 | 0.3 | M | Sodium | 0.3 | M | Tris-HCl | 8.0 |
| | | | chloride | | | | |
| G8 | 0.745 | M | Sodium | 0.3 | M | Tris-HCl | 8.0 |
| | | | chloride | | | | |
| G9 | 1.49 | M | Sodium | 0.3 | M | Tris-HCl | 8.0 |
| | | | chloride | | | | |
| G10 | 2.975 | M | Sodium | 0.3 | M | Tris-HCl | 8.0 |
| | | | chloride | | | | |
| G11 | 1.49 | M | Sodium | 0.595 | M | MOPS | 7.0 |
| | | | chloride | | | | |
| G12 | | | | 0.595 | M | SPG | 4.0 |
| H1 | | | | 0.3 | M | MES/Bis-Tris | 6.0 |
| H2 | | | | 0.3 | M | MES/Imidazole | 6.5 |
| H3 | | | | 0.3 | M | Bis-Tris/PIPES | 6.5 |
| H4 | | | | 0.3 | M | MOPS/Bis-Tris propane | 7.0 |
| H5 | | | | 0.3 | M | Phosphate/Citrate | 7.5 |
| H6 | | | | 0.3 | M | MOPS/Sodium HEPES | 7.5 |
| H7 | | | | 0.3 | M | BICINE/Tris | 8.5 |
| H8 | 0.595 | M | Sodium | 0.3 | M | Imidazole | 7.5 |
| | | | chloride | | | | |
| H9 | 0.595 | M | Sodium | 0.745 | M | Imidazole | 7.5 |
| | | | chloride | | | | |
| H10 | 0.595 | M | Sodium | 1.49 | M | Imidazole | 7.5 |
| | | | chloride | | | | |
| H11 | 0.595 | M | Sodium | 2.085 | M | Imidazole | 7.5 |
| | | | chloride | | | | |
| H12 | 0.595 | M | Sodium | 2.975 | M | Imidazole | 7.5 |
| | | | chloride | | | | |

TABLE 8
Concentrations of pre-diluted additives in screening:

| | C1 | C2 | C3 | C4 | C1 | C2 | C3 | C4 |
|---|---|---|---|---|---|---|---|---|
| ADDITIVE | [mM] | [mM] | [mM] | [mM] | [%] | [%] | [%] | [%] |
| L-Proline | 416.7 | 208.3 | 104.2 | 52.1 | | | | |
| Glycerol | 833.3 | 416.7 | 208.3 | 104.2 | | | | |
| Calcium chloride | 83.3 | 41.7 | 20.8 | 10.4 | | | | |
| Ammonium sulfate | 833.3 | 416.7 | 208.3 | 104.2 | | | | |
| Urea | 416.7 | 208.3 | 104.2 | 52.1 | | | | |
| Guanidine hydrochloride | 833.3 | 416.7 | 208.3 | 104.2 | | | | |
| Potassium acetate | 416.7 | 208.3 | 104.2 | 52.1 | | | | |
| Lithium acetate | 833.3 | 416.7 | 208.3 | 104.2 | | | | |
| Ammonium chloride | 833.3 | 416.7 | 208.3 | 104.2 | | | | |
| Sodium sulfate | 833.3 | 416.7 | 208.3 | 104.2 | | | | |
| Barium chloride | 41.7 | 20.8 | 10.4 | 5.2 | | | | |
| 1,2,3-hexanetriol | 416.7 | 208.3 | 104.2 | 52.1 | | | | |
| Polypropylene glycol | 208.3 | 104.2 | 52.1 | 26.0 | | | | |
| 400 | | | | | | | | |
| D-(+)-Trehalose | 416.7 | 208.3 | 104.2 | 52.1 | | | | |
| L-Glutathione reduced, | 4.2 | 2.1 | 1.0 | 0.5 | | | | |
| L-Glutathione oxidized | | | | | | | | |
| Ethylene- | 41.7 | 20.8 | 10.4 | 5.2 | | | | |
| diaminetetraacetic | | | | | | | | |
| acid disodium salt | | | | | | | | |
| Benzamidine | 416.7 | 208.3 | 104.2 | 52.1 | | | | |
| hydrochloride | | | | | | | | |
| LysoFos Choline 10 | 26.7 | 13.3 | 6.7 | 3.3 | | | | |
| BRIJ ® 35 | 0.3 | 0.2 | 0.1 | 0.0 | | | | |
| n-Octyl-β-D-glucoside | 60.0 | 30.0 | 15.0 | 7.5 | | | | |
| ANAPOE ® NID-P40 | 1.0 | 0.5 | 0.3 | 0.1 | | | | |
| FOS-Choline ®-14 | 0.4 | 0.2 | 0.1 | 0.1 | | | | |
| CHAPS | 26.7 | 13.3 | 6.7 | 3.3 | | | | |
| ZWITTERGENT 3-16 | 0.2 | 0.1 | 0.1 | 0.0 | | | | |
| Sodium cholate hydrate | 38.3 | 19.2 | 9.6 | 4.8 | | | | |
| Sodium deoxycholate | 13.3 | 6.7 | 3.3 | 1.7 | | | | |
| Sodium dodecyl sulfate | 27.3 | 13.7 | 6.8 | 3.4 | | | | |
| 2-Propanol | | | | | 10 | 5 | 2.5 | 1.25 |
| Acetonitrile | | | | | 10 | 5 | 2.5 | 1.25 |
| Acetone | | | | | 10 | 5 | 2.5 | 1.25 |

### 11.3.2 HTRF Procedure

Streptavidin-d2 (SA-d2, 610SADLA, Cisbio) and pAb anti-human IgG-Tb cryptate (lgG-Tb, 61HFCTAA, Cisbio) were diluted to CSA-d2=1500 ng/ml and ClgG-Tb=150 ng/ml in HTRF 5 96-well low volume white plate (66PL96100) in 9 μL Terbium Buffer (61DB10RDF) final. The transglutaminase conjugation reaction was diluted in PBS pH 7.4 (Gibco) to yield 10 nM antibody stock solution and 1 μL of the dilution was added to the HTRF reagent mix. It was pipetted carefully to avoid bubbles in the wells. The plate was sealed with an adhesive strip to avoid evaporation and incubated at room temperature for 4 h. The background fluorescence 10 was detected by two blank measurements with PBS as sample volume. Detection of the TR-FRET signal was performed with the EnVision 2104 multiplate reader using the excitation filter UV2 (320 nm) and the emission filters APC (665 nm) and Europium (615 nm). Data processing was performed with EnVision Workstation version 1.12.

### 11.3.3 Data Reduction

Step 1: Calculate the ratio as shown in equation Eq. 1 below for each well. This calculation minimizes well to well variations because the background noise of the donor is normalized for each well.

$$ratio = 1 {0}^{4} * \frac{{Emission}_{620 nm}}{{Emission}_{665 nm}} \qquad Eq . 1$$

Step 2: The calculation given in equation Eq. 2 below normalizes the measurement for each well so that plate-to-plate variation is minimized.

$$Δ F = \frac{{ratio}_{sample} - {ratio}_{negative control}}{{ratio}_{negative control}} \qquad Eq . 2$$

11.3.4 mTG-Mediated Drug-Linker Conjugation and Titration Experiments

Typically, Herceptin/trastuzumab was conjugated with linker-payload Gly3-VC-MMAE 1 (G3-MMAE, Moradec LLC) in different buffer systems using increasing concentration of MTG_1 with otherwise constant reaction conditions. DAR was determined using reversed-phase chromatography as described in 11.3.5.

The following reaction conditions were used: 5 μL of 5× concentrated buffer were mixed with 15 μL deionized water. 1.04 μL 120 mg/mL Herceptin was added to a yield 5 mg/ml final, followed by the addition of 1.46 μL 10 mM Gly3-VC-MMAE to yield a final concentration of 583.3 μM. MTG_1 was diluted in water to 200, 150, 100, 50 and 25 U/mL. 2.5 μL of the MTG_1 predilution were added to reagent mixes prepared beforehand. The conjugation reaction was performed for 16 h at 37° C. while shaking at 600 rpm. The reaction was stopped by adding 2.5 μL transglutaminase inhibitor B102 (Zedira).

### 11.3.5 Drug-Antibody-Ratio (DAR) Analytics

Reversed-phase HPLC was performed using a PLRP-S 1000 Å (50×2.1 mm, 8 μm) column (Agilent) run at 1.0 mL/min and 65° C. 5-7.5 μg antibody or ADC were injected and chromatography performed running a 7.5 minute linear gradient from 30-45% solvent B over A (solvent A: Lichrosolv H₂O+0.1% TFA; solvent B: 100% AcN+0.1% TFA) with monitoring absorbance at 214 nm wavelength.

### MTG Sequences

Herein the mTG enzymes with the following names have the respective amino acid sequences shown in the following:
MTG_proenzyme (=SEQ ID NO 44).
MTG_1 (activated) (=SEQ ID NO: 45)
MTG_2 (activated) (=SEQ ID NO: 73)
MTG_3 (activated) (=SEQ ID NO: 48)
MTG_4 (activated) (=SEQ ID NO: 49)

Also the following mTG enzyme from S. mobaraensis can be used in the methods of the invention:

```
MTG_5 (from S. mobaraensis) (=SEQ ID NO: 81):
        DSDDRV TPPAEPLDRM PDPYRPSYGR AETVVNNYIR KWQQVYSHRD GRKQQMTEEQ
REWLSYGCVG VTWVNSGQYP TNRLAFASFD EDRFKNELKN GRPRSGETRA EFEGRVAKES
FDEEKGFQRA REVASVMNRA LENAHDESAY LDNLKKELAN GNDALRNEDA RSPFYSALRN
TPSFKERNGG NHDPSRMKAV IYSKHFWSGQ DRSSSADKRK YGDPDAFRPA PGTGLVDMSR
DRNIPRSPTS PGEGFVNFDY GWFGAQTEAD ADKTVWTHGN HYHAPNGSLG AMHVYESKFR
NWSEGYSDFD RGAYVITFIP KSWNTAPDKV KQGWP
MTG_6 (from S. mobaraensis) (=SEQ ID NO: 82):
        DSDDRV TPPAEPLDRM PDPYRPSYGR AETVVNNYIR KWQQVYSHRD GRKQQMTEEQ
REWLSYGCVG VTWVNSGQYP TNRLAFASFD EDRFKNELKN GRPRSGETRA EFEGRVAKES
FDEEKGFQRA REVASVMNRA LENAHDESAY LDNLKKELAN GNDALRNEDA RSPFYSALRN
TPSFKERNGG NHDPSRMKAV IYSKHFWSGQ DRSSSADKRK YGDPDAFRPA PGTGLVDMSR
DRNIPRSPTS PGEGFVNFDY GWFGAQTEAD ADKTVWTHGN HYHAPNGSLG AMHVYESKFR
NWSEGYSDFD RGAYVITFIP KSWNTAPDKV
MTG_7 (from S. mobaraensis) (=SEQ ID NO: 83):
FRAPDSDDRV TPPAEPLDRM PDPYRPSYGR AETVVNNYIR KWQQVYSHRD GRKQQMTEEQ
REWLSYGCVG VTWVNSGQYP TNRLAFASFD EDRFKNELKN GRPRSGETRA EFEGRVAKES
FDEEKGFQRA REVASVMNRA LENAHDESAY LDNLKKELAN GNDALRNEDA RSPFYSALRN
TPSFKERNGG NHDPSRMKAV IYSKHFWSGQ DRSSSADKRK YGDPDAFRPA PGTGLVDMSR
DRNIPRSPTS PGEGFVNFDY GWFGAQTEAD ADKTVWTHGN HYHAPNGSLG AMHVYESKER
NWSEGYSDFD RGAYVITFIP KSWNTAPDKV KQGWP
MTG_8 (from S. mobaraensis) (=SEQ ID NO: 84):
FRAPDSDDRV TPPAEPLDRM PDPYRPSYGR AETVVNNYIR KWQQVYSHRD GRKQQMTEEQ
REWLSYGCVG VTWVNSGQYP TNRLAFASFD EDRFKNELKN GRPRSGETRA EFEGRVAKES
FDEEKGFQRA REVASVMNRA LENAHDESAY LDNLKKELAN GNDALRNEDA RSPFYSALRN
TPSFKERNGG NHDPSRMKAV IYSKHFWSGQ DRSSSADKRK YGDPDAFRPA PGTGLVDMSR
DRNIPRSPTS PGEGFVNFDY GWFGAQTEAD ADKTVWTHGN HYHAPNGSLG AMHVYESKFR
NWSEGYSDFD RGAYVITFIP KSWNTAPDKV KQGWPLE
```

### Example 12: mTG Recognition Tags

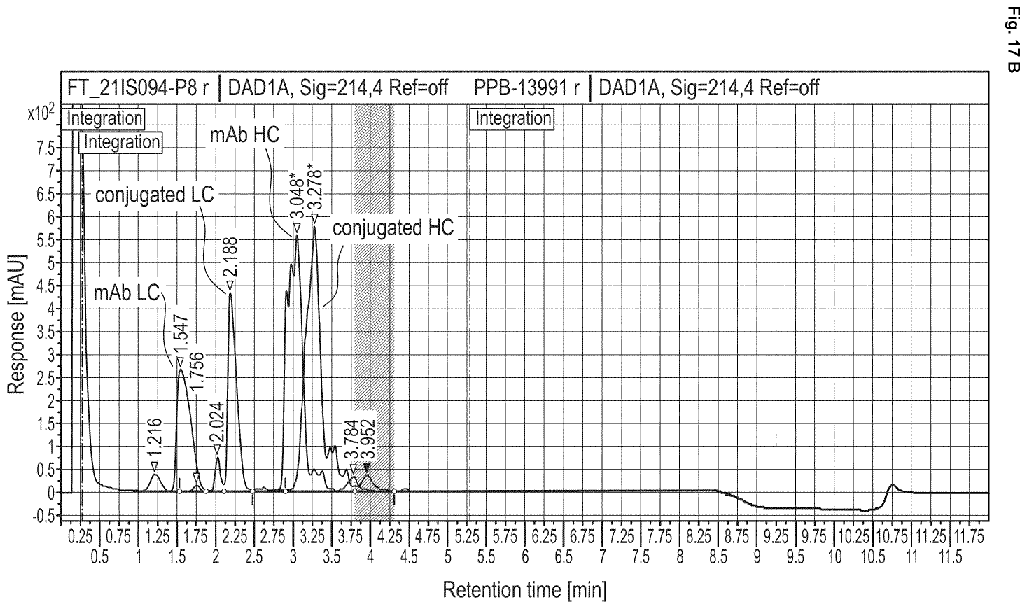

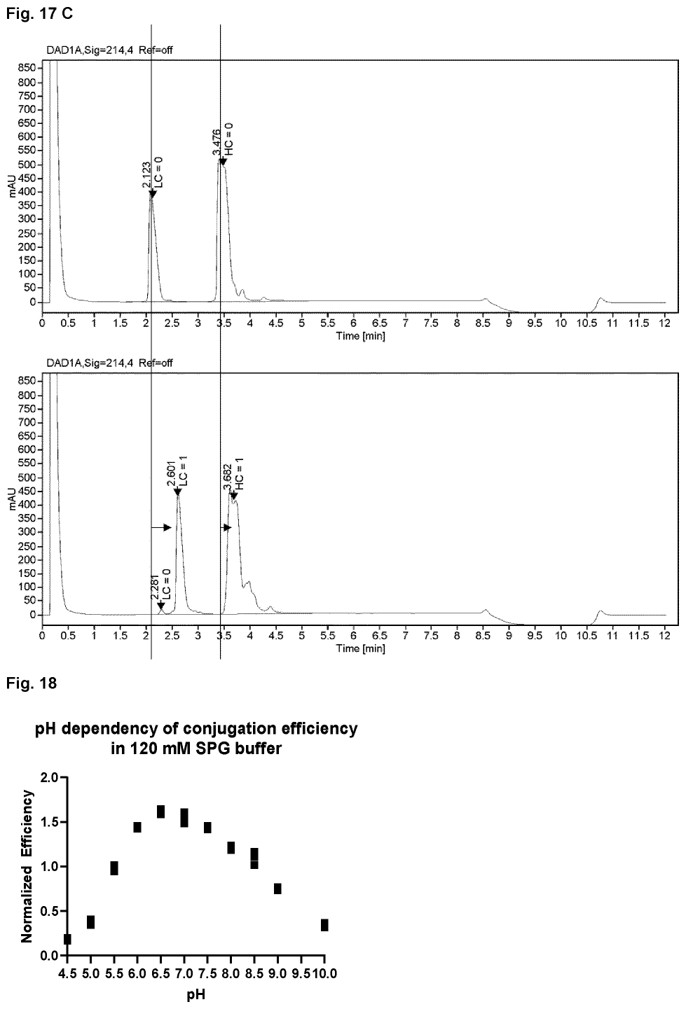

To achieve even higher DARs with the mTG conjugation method described above (i.e. previously two drugs had been conjugated at position Q295 (Eu numbering) in each of both heavy chains of mAbs), certain peptide motifs were genetically fused to the mAb of interest to enable further conjugation at these motifs. Some examples of such peptide tags have been described in the prior art. However, unexpectedly, new and functional additional peptide sequences were identified (e.g. GGTLQSPP and TLQS) that can be used in a similar manner. Conjugation of antibodies fused with such peptide motifs using mTG from ladakanum with resulted in ADCs with DAR of around 4 (see FIG. 17).

### 12.1 Identification and Evaluation of MTG Recognition Tags

### Conjugation of mAb Tag1 Antibody

An antibody that comprises the amino acid sequence “TLQS” within the light chains of the antibody was recombinantly expressed using pTT5-based expression vectors and the HEK293 expression system (Gibco™, Thermo Fisher Scientific Inc.) followed by purification using protein A chromatography. The antibody was conjugated using microbial transglutaminase (T001, Zedira GmbH) with drug-linker 4M.

Conjugation was performed using 34.4 μM mAb, 344 UM G3-MMAE and 6 U/ml MTG (purchased from Zedira, Germany) 25 mM in Tris-HCl, pH 8.0, 150 mM sodium chloride and incubated at 37° C. for 16 h. ADCs were separated from G3-MMAE 4M and possible high molecular weight species (HMWS) via size exclusion chromatography (SEC). SEC was carried out using a Superdex 200 increase 10/300 column with PBS, pH 7.0 as running buffer. Fractionated samples containing the ADC material were pooled and concentrated to >1 mg/ml using Amicon Ultra 15 50K centrifugal (Millipore).

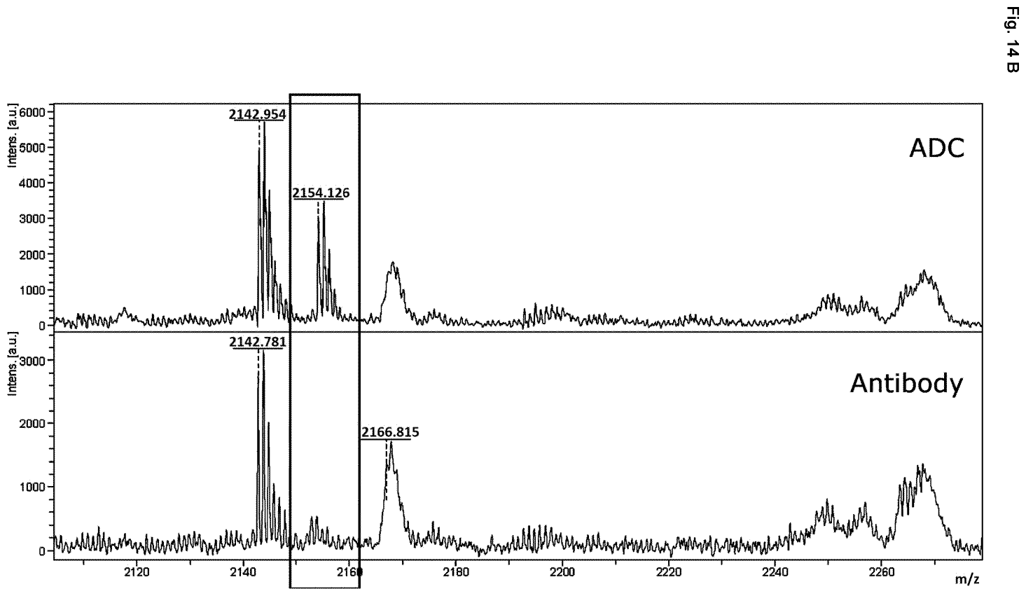

ADCs were analyzed using hydrophobic interaction chromatography (HIC) and mass spectrometry (MS) revealing a drug-to-antibody ratio (DAR) of 1.7 (see FIG. 13).

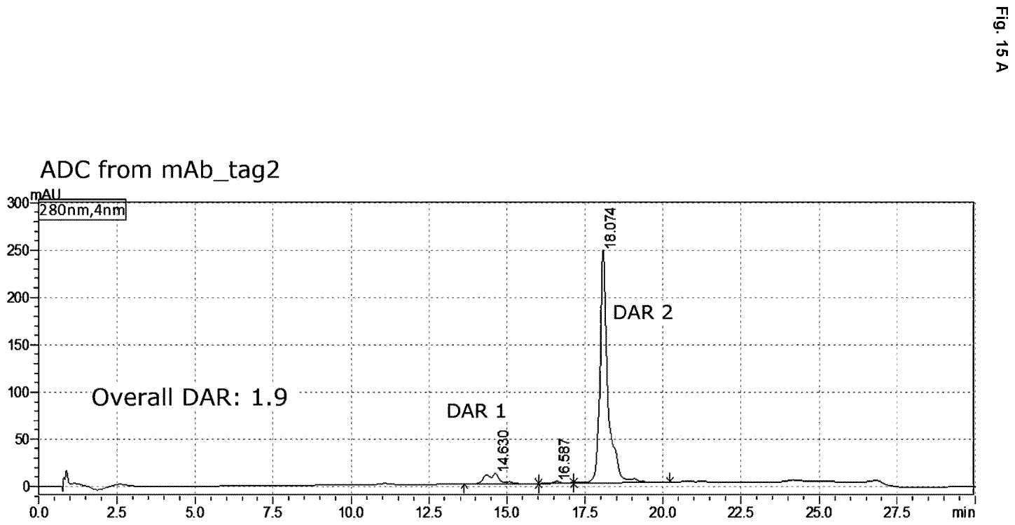

Conjugated and unconjugated antibodies were digested via chymotryptic digestion using the Smart Digest Kit (Thermo Fisher Scientific) and subsequently analyzed by MALDI-MS using a Bruker Ultraflex MALDI TOF/TOF mass spectrometer with a a-Cyano-4 hydroxy-cinamic acid matrix. Comparison of detected masses revealed a peak present for the mAb but not present for the ADC at 877.368 Da (see FIG. 14). This mass matches the theoretical mass of the light chain peptide QSGVPSRF (876.968 Da). Comparison of detected masses revealed a peak present for the ADC but not present for the mAb at 2154.126 Da (see FIG. 14). This mass closely matches the theoretical mass of the light chain peptide QSGVPSRF conjugated with one Gly3-VC-MMAE peptide (2154.968 Da). These results confirm that the Gly3-VC-MMAE linker-payload could successfully be conjugated to the peptide QSGVPSRF of the antibody light chain.

### Conjugation of mAb Tag2, mAb Tag3 and mAb Tag4 Antibodies

The above identified peptide was located in the light chain of the antibody as part of the sequence YTSTLQSGVPS. Its core sequence TLQSG was fused to the sequence of a trastuzumab-derived HER2-binding antibody replacing the lysine 447 of the heavy chain (mAb_tag2). The antibody was recombinantly expressed using the Expi293 expression system (Gibco™) using pTT5-based plasmids and purified using protein A and size-exclusion chromatography. Mass spectrometry revealed partial truncation of the C-terminal QSG peptide. Further antibodies were produced using the same procedures with the sequences GGTLQSG (mAb_tag3) and LC-GGTLQSPP (mAb_tag4) fused to the light chain C-termini. Mass spectrometry revealed no truncation of the peptide tag for mAb_tag4.

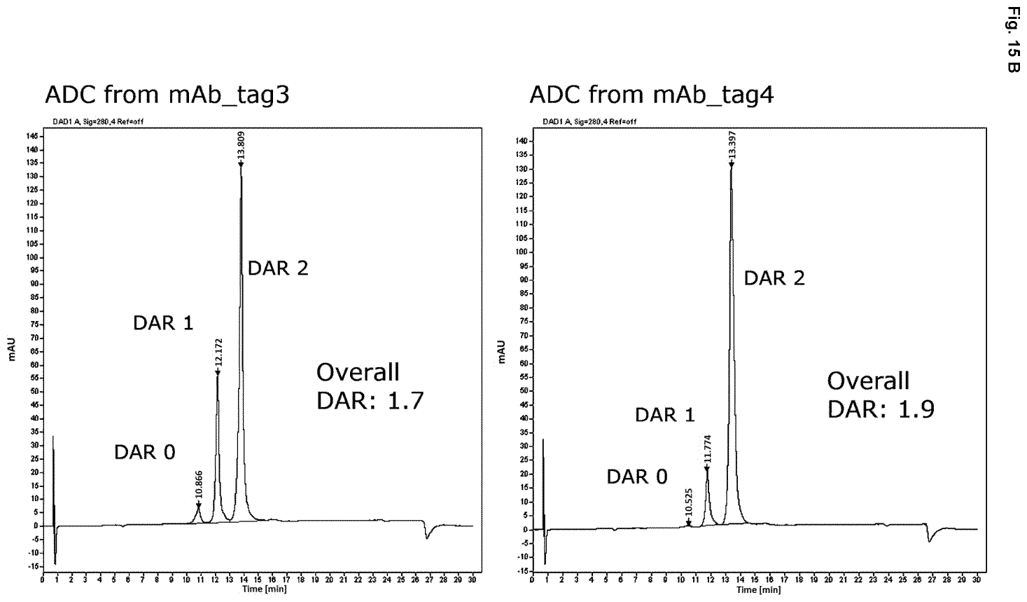

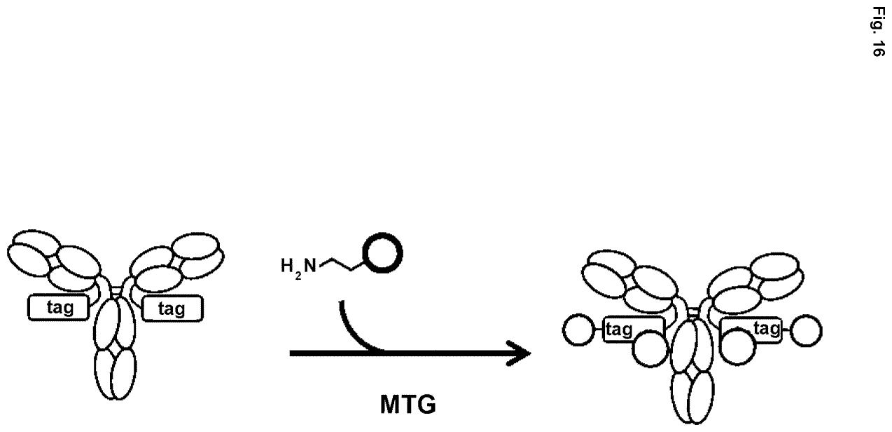

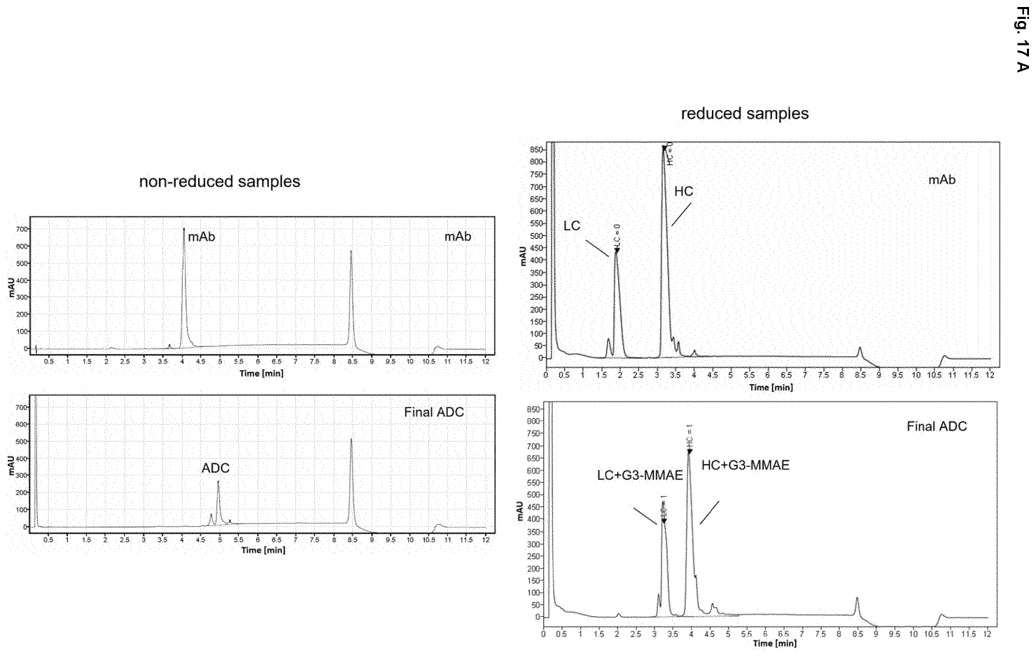

The antibodies mAb tag2, mAb tag3 and mAb tag4_were conjugated and purified analogously as described above using an mTG enzyme from S. mobaraensis for the antibody that comprises the “TLQS” peptide. Drug-to-antibody ratios (DAR) were assessed using HIC and MS and revealed DAR values of 1.9, 1.7 and 1.9 for mAb_tag2, _tag3 and_tag4, respectively (see FIG. 15).

12.2 Conjugation of mAb Equipped with Recognition Tag (mAb_Tag5 and mAb7-M) Using Technology for Native mAb Conjugation

Antibodies comprising a glutamine-containing recognition tag were conjugated using native mAb conjugation via mTG from S. ladakanum in combination with reaction conditions determined as suitable for simultaneous conjugation of four drug-linkers per antibody.

The tag sequence GGTLQSPP was fused to the light chain C-termini sequence of a C_{D}20-specific mAb derived from rituximab, mAb_tag5, as well as ceacam-5 specific mAb7-M. Antibodies were recombinantly expressed using an Expi293 expression system (Gibco™) using pTT5-based plasmids and purified using protein A and size-exclusion chromatography. Drug-linker 4M or 1M were conjugated to the antibodies, followed by SEC purification and DAR determination using RP-HPLC as described above.

For this, the mAbs were buffer exchanged to 24 mM HEPES, pH 7.0 using HiTrap Desalting columns in combination with an Äkta liquid chromatography (LC) system (Cytiva). A conjugation mixture was set up as follows: 5 mg/ml mAb, 5 molar equivalents of drug-linker 4M or 1M per conjugation site, 20 U/ml MTG_1, 4% DMSO, 24 mM HEPES, pH 7.0. The reaction was carried out at 37° C. for 18 h. ADCs were separated from drug-linker and mTG via size exclusion chromatography (SEC). Prior to SEC purification, NaCl concentration of the samples was adjusted to 100 mM using a 5 M NaCl stock solution. SEC was carried out using a HiLoad Superdex 200 10/30 increase column with 10 mM Histidine, 100 mM NaCl, 3% Trehalose, pH 7.0 as running buffer. Fractionated samples containing the ADC material were pooled and concentrated to >1 mg/ml using Amicon Ultra 15 50K centrifugal (Millipore). Drug-to-antibody ratio and purity were assessed using reversed phase (RP) chromatography.

Efficient conjugation to both the light and heavy chain resulting in drug-to-antibodies ratios of 3.9 was observed in all investigated cases (see FIG. 17), demonstrating successful conjugation of drug-linkers to Q295 in the heavy chain despite the naturally occurring glycan at N297 plus additional drug-linker attachment to the LC-fused glutamine-tag GGTLQSPP.

Antibody Sequences used in the conjugation experiments:

>mAb_tag2_LC (trastuzumab)
(SEQ ID NO: 66)
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC
>mAb_tag2_HC (trastuzumab-HC-tag)
(SEQ ID NO: 67)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPPKSC
DKTHTCPPCPAPELLGGPSVFLFPPKPKDTLMISRTPEVTCVVVDVSHEDPEVKFNW
YVDGVEVHNAKTKPREEAYNSTYRVVSVLTVLHQDWLNGKEYKCKVSNKALPAPI
EKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSP
GTLQSG
>mAb_tag3_LC (trastuzumab-LC-tag1)
(SEQ ID NO: 68)
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECGGTLQSG
>mAb_tag4_LC (trastuzumab-LC-tag2)
(SEQ ID NO: 69)
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKVEIKRTVAAPSV
FIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGECGGTLQSPP
>mAb_tag3&4_HC (trastuzumab HC)
(SEQ ID NO: 70)
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
RTHTCPPCPAPELLGGPSVFLFPPKPKDTLMISRTPEVTCVVVDVSHEDPEVKFNWY
VDGVEVHNAKTKPREEAYNSTYRVVSVLTVLHQDWLNGKEYKCKVSNKALPAPIE
KTISKAKGQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKSLSLSPG
>mAb_tag5_HC (~rituximab HC = SEQ ID NO: 40)
(SEQ ID NO: 40)
QVQLQQPGAELVKPGASVKMSCKASGYTFTSYNMHWVKQTPGRGLEWIGAIYPGN
GDTSYNQKFKGKATLTADKSSSTAYMQLSSLTSEDSAVYYCARSTYYGGDWYFNV
WGAGTTVTVSAASTKGPSVFPLAPSSKSTSGGTAALGCLVKDYFPEPVTVSWNSGA
LTSGVHTFPAVLQSSGLYSLSSVVTVPSSSLGTQTYICNVNHKPSNTKVDKRVEPKS
CDKTHTCPPCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLS
PGK
>mAb_tag5_LC (rituximab-LC-tag)
(SEQ ID NO: 71)
QIVLSQSPAILSASPGEKVTMTCRASSSVSYIHWFQQKPGSSPKPWIYATSNLASGVP
VRESGSGSGTSYSLTISRVEAEDAATYYCQQWTSNPPTFGGGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGECGGTLQSPP
>mAb7-M_HC (ceacam-HC)
(SEQ ID NO: 72)
EVQLQESGPGLVKPSQTLSLTCTVSDGSVSRGGYYLTWIRQHPGKGLEWIGYIYYSGS
TYFNPSLRSRVTMSVDTSKNQFSLKLSSVTAADTAVYYCARGIAVAPFDYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKRVEPKSCDKTHTCPP
CPAPEAAGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPG
>mAb7-M_LC (ceacam LC tag = SEQ ID NO: 36)
(SEQ ID NO: 36)
EIVLTQSPATLSVSPGERATLSCRTSQSVRSNLAWYQQKPGQAPRLLIYAASTRATGI
PARFSGSGSGTEFTLTISSLQSEDFAVYYCQQYTNWPFTFGPGTKVDIKRTVAAPSVF
IFPPSDEQLKSGTASVVCLLNNFYPREAKVQWKVDNALQSGNSQESVTEQDSKDST
YSLSSTLTLSKADYEKHKVYACEVTHQGLSSPVTKSFNRGECGGTLQSPP

1. Method for producing an antibody-linker-conjugate comprising the steps:
(1) providing an antibody that optionally comprises an amino acid sequence selected from the group consisting of GGTLQSPP, TLQSG, TLQSPP, GGTLQSG and TLQSA and more preferably the amino acid sequence TLQSPP or GGTLQSPP (most preferably the amino acid sequence GGTLQSPP) in at least one and preferably both of its light chain constant regions (CL) and/or in at least one and preferably both of its heavy chain constant regions (CH);
(2) mixing together in a reaction buffer at least the following components:
(a) said antibody provided in step (1);
(b) a microbial transglutaminase, preferably a transglutaminase comprising the amino acid sequence as defined in SEQ ID NO:s 44, 47, 48, 49, 45, 73, 81, 82, 83 or 84 and preferably comprising the amino acid sequence of SEQ ID NO 45; and
(c) a linker or a therapeutic agent that comprises an H₂N-moiety capable of reacting with the antibody from step (1) in the presence of said transglutaminase and wherein the linker is preferably a drug-linker where said linker is covalently attached to a drug;
(3) separating the antibody-linker-conjugate produced in step (2) from unreacted linker and from said transglutaminase preferably by subjecting said mixture from step (2) to a size-exclusion chromatography.

2. The method of claim 1, wherein said reaction buffer is a reaction buffer that is between 0% and 7% DMSO, between 20 and 30 mM HEPES (preferably 24 mM HEPES) and pH of between 6.8 and 7.4 (preferably pH 7); or a reaction buffer that comprises between 1% and 10% DMSO, between 5 and 100 mM HEPES, and comprises less than 150 mM NaCl and has a pH of between 6 and 8; or is a reaction buffer that comprises between 1% and 10% DMSO, between 5 and 60 mM HEPES, and comprises less than 10 mM NaCl (preferably no NaCl) and has a pH of between 6 and 7.5; or
a reaction buffer that comprises between 5 and 100 mM HEPES, and comprises less than 150 mM NaCl and has a pH of between 6 and 8; or
is a reaction buffer that comprises between 5 and 60 mM HEPES, and comprises less than 10 mM NaCl (preferably no NaCl) and has a pH of between 6 and 7.5.

3. The method according to claim 1, wherein said antibody comprises a glutamine at position 295 of the heavy chain of the antibody, whereby the antibody is a monoclonal antibody and wherein Eu numbering is used for defining the position of said glutamine at position 295 and/or wherein the antibody comprises an amino acid sequence selected from the group consisting of GGTLQSPP, GGTLQSG and TLQSA at the C-terminus of the heavy- and/or light chain of the antibody; and
wherein the reaction mixture comprises less than 10 mM NaCl and wherein the reaction buffer comprises one or more of the following buffers adjusted to a pH of between 7 and pH 8.8:
BICINE, BICINE/Tris, Tris-HCl, HEPES and/or Tricine.

4. The method according to claim 1, wherein said linker is a linker having the formula 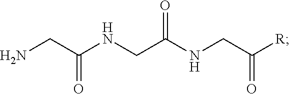
wherein R is the remainder of the linker which may also include a therapeutic agent or detectable label.

5. The method of claim 4, wherein in step (2) the mixture comprises the following drug-linker:

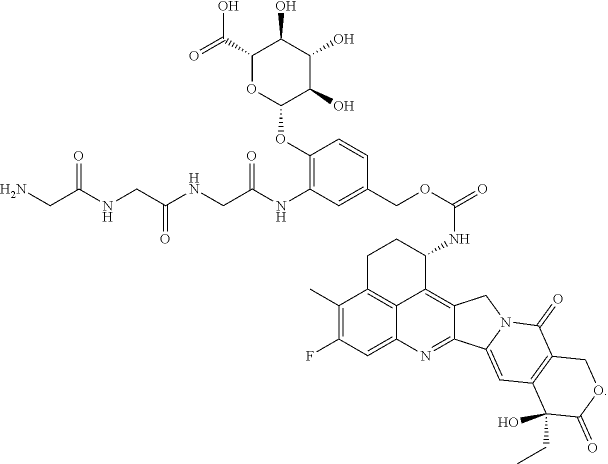 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(NC(=O)CNC(=O)CNC(=O)CN)c1)CC3]

6. The method according to claim 1, wherein the mixture in step (2) comprises a molecular excess of linker or drug-linker or therapeutic agent, preferably at least 2, and more preferably at least 5 molar equivalents of linker or drug-linker or therapeutic agent, respectively, per conjugation site, wherein a conjugation site is a sequence GGTLQSPP, TLQSG, TLQSPP, GGTLQSG or TLQSA comprised in the light chain constant regions (CL) and/or in the heavy chain constant region (CH) of said antibody; and/or wherein a conjugation site is a glutamine at position 295 of the heavy chain of the antibody, whereby the antibody is a monoclonal antibody and wherein Eu numbering is used for defining the position of said glutamine at position 295.

7. The method according to claim 1, wherein the antibody is a monoclonal antibody and preferably an anti-CEACAM5 antibody and/or
wherein the drug is a growth inhibitory agent as defined in (a), (b) or (c) as specified in the following:
(a) the growth inhibitory agent is a cytotoxic drug or a radioactive moiety.
(b) the growth inhibitory agent is selected from a group consisting of chemotherapeutic agents, enzymes, antibiotics, toxins such as small molecule toxins or enzymatically active toxins, toxoids, vincas, taxanes, maytansinoids or maytansinoid analogs, tomaymycin or pyrrolobenzodiazepine derivatives, cryptophycin derivatives, leptomycin derivatives, auristatin or dolastatin analogs, prodrugs, topoisomerase I inhibitors, topoisomerase II inhibitors, DNA alkylating agents, anti-tubulin agents, CC-1065 and CC-1065 analogs;
(c) the growth inhibitory agent is exatecan.

8. The Antibody-linker conjugate producible according to the method of claim 1, wherein the linker is preferably a linker or drug linker as defined in the following: 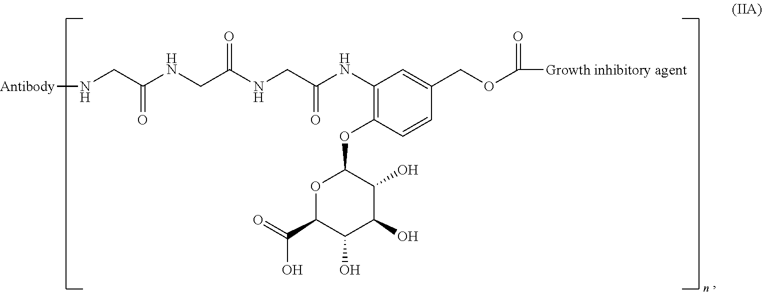
wherein n is a number of [(linker)-(growth inhibitory agent)] moieties covalently linked to the antibody; or 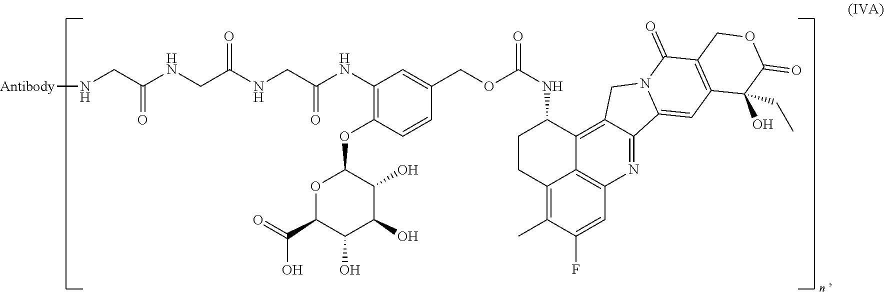
wherein n is a number of [(linker)-(exatecan)] moieties covalently linked to the antibody; and wherein n is preferably between 1 and 10 and more preferably 4.

9. The Antibody-linker conjugate of claim 8, wherein the linker is a drug-linker.

10. The method of claim 4, wherein in step (2) the mixture comprises the following drug-linker:

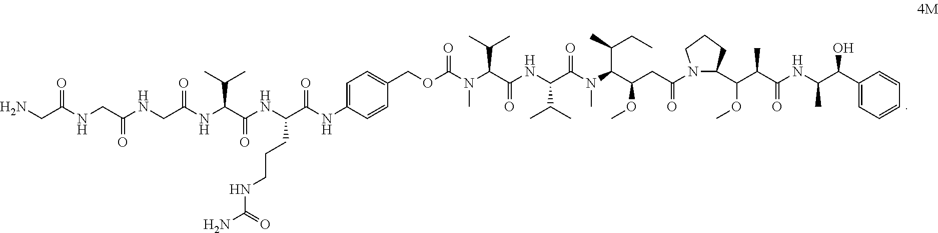 [SMILES: CC[C@H](C)[C@@H]([C@@H](CC(=O)N1CCC[C@H]1C(OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CNC(=O)CNC(=O)CN)C(C)C)cc1)C(C)C]

11. The method according to claim 1, wherein the glutamine of the amino acid sequence selected from the group consisting of GGTLQSPP, TLQSG, TLQSPP, GGTLQSG and TLQSA of said antibody reacts in step (2) of the method in the presence of said microbial transglutaminase to form a covalent bond with said H₂N-moiety of the linker.

12. The method according to claim 1, wherein the antibody is a monoclonal antibody.

13. The method according to claim 1, wherein the microbial transglutaminase is a microbial transglutaminase from S. ladakanum or from S. mobaraensis.

14. The method according to claim 1, wherein the reaction mixture obtained in step (2) is incubated at a temperature between 20° C. and 38° C. until the antibody-linker-conjugate has formed and can be separated in method step (3).

15. The method according to claim 1, wherein the reaction buffer comprises one or more of the following buffers adjusted to a pH of between 7 and pH 8.8: BICINE, BICINE/Tris, Tris-HCl, HEPES and/or Tricine.

16. The method according to claim 1, wherein the reaction buffer comprises Tris-HCl adjusted to a pH of between 7.7 and 8.5: or comprises BICINE and/or BICINE/Tris adjusted to a pH of between 7.8 and 8.8: or comprises Tricine adjusted to a pH of between 7.8 and 8.0.

17. The method according to claim 1, wherein the reaction buffer comprises HEPES in an amount of between 5 mM and 100 mM.

18. The method according to claim 1, wherein the reaction buffer is a reaction buffer that comprises between 0% and 10% DMSO, between 5 and 60 mM HEPES, and comprises less than 100 mM NaCl (preferably no NaCl) and has a pH of between 6 and 7.5;
said antibody further comprises a GGTLQSPP, TLQSG, TLQSPP, GGTLQSG and TLQSA sequence in both light chain constant regions (CL) and/or in both heavy chain constant regions (CH); and
said linker is a linker having the formula 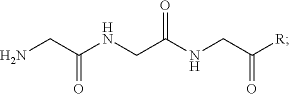
wherein R is the remainder of the linker and may optionally also comprise a drug.

19. The method according to claim 1, wherein said reaction buffer comprises less than 150 mM NaCl, preferably less than 100 mM NaCl, more preferably less than 75 mM NaCl, even more preferably 5 mM NaCl or less, most preferably wherein said reaction buffer comprises no NaCl.

20. The method according to claim 1, wherein the total amount of all buffering agents that are comprised in said reaction buffer is lower than 160 mM and preferably lower than 155 mM.

21. The method according to claim 1, wherein the total amount of all buffering agents that are comprised in said reaction buffer is lower than 70 mM.

22. The method according to claim 1, wherein the total amount of all buffering agents that are comprised in said reaction buffer is lower than 155 mM; and wherein the reaction buffer comprises less than 10 mM NaCl and wherein the total amount of microbial transglutaminase is between 2.5 and 12 U/ml.

23. The method according to claim 1, wherein the total amount of all buffering agents that are comprised in said reaction buffer is lower than 70 mM; and wherein the reaction buffer comprises less than 50 mM NaCl; wherein the total amount of microbial transglutaminase is between 2.5 and 12 U/ml and wherein the pH of the reaction buffer is between pH 5.9 and pH 8.1.

24. The method according to claim 1, wherein the antibody is trastuzumab, rituximab or labetuzumab; or
wherein said antibody comprises the amino acid Q295 in its heavy chain constant regions (CH), wherein Eu numbering is used for defining the position of said glutamine at position 295; or
wherein said antibody comprises the amino acid Q295 in its heavy chain constant regions (CH), wherein Eu numbering is used for defining the position of said glutamine at position 295; and wherein said antibody is an IgG monoclonal antibody.

25. The method according to claim 1, wherein said reaction buffer comprises at least one buffering agent selected from the group consisting of Imidazole, HEPES, BICINE, Tris-HCl, MOPS/Bis-Tris propane and MOPS/Sodium HEPES, and wherein the buffering agent concentration is selected within the range of 10-160 mM, preferably wherein the buffering agent is HEPES at a concentration of between 10 and 30 mM, more preferably at a concentration of 24 mM.

26. The method according to claim 1, wherein said reaction buffer has a pH of between 6.5 and 8.7, more preferably a pH of 6.8 to 8.2, most preferably a pH of about 7; or wherein said reaction buffer has a pH of between about 7 and 8.5 and wherein the reaction buffer comprises between 20 and 120 mM of at least one of the following buffering agents: HEPES, Tris-HCl, MOPS/NaHEPES, BICINE, BICINE/Tris, Imidazole and Tricine and comprises less than 10 mM NaCl.

27. The method according to claim 1, wherein said reaction buffer comprises between about 0.5% and 8% DMSO and preferably about between 4% and 7% DMSO, and most preferably a maximum of 5% DMSO.

28. The method according to claim 1, wherein said reaction buffer comprises said transglutaminase wherein said transglutaminase comprises the amino acid sequence of SEQ ID NO: 45 and wherein said microbial transglutaminase is comprised in the mixture in step (2) of the method at a concentration of between 5 U/mL and 25 U/mL and preferably at a concentration of between 10 U/mL and 20 U/mL.

29. The method according to claim 1, wherein said reaction buffer comprises no sodium chloride and further comprises about 24 mM HEPES, wherein said reaction buffer has a pH of about 7.5 and wherein the microbial transglutaminase concentration is about 12.5 U/mL.

30. The method according to claim 1, wherein said reaction buffer comprises no sodium chloride and further comprises about 24 mM HEPES, wherein said reaction buffer has a pH of about 7 and comprises about 4% DMSO, wherein the microbial transglutaminase concentration in the mixture in step (2) of the method is about 20 U/mL.

31. The method according to claim 1, wherein said reaction buffer comprises no sodium chloride and further comprises about 24 mM HEPES, wherein said reaction buffer has a pH of about 7 and comprises about 7% DMSO, wherein the microbial transglutaminase concentration in the mixture of step (2) of the method is about 20 U/mL.

32. The method according to claim 1, wherein said antibody comprises the amino acid sequence TLQSG, GGTLQSPP or GGTLQSG at the C-terminus of its light chain constant regions (CL) and the amino acid Q295 in its heavy chain constant regions (CH), wherein Eu numbering is used for defining the position of said glutamine at position 295.

33. The method according to claim 1, wherein the drug antibody ratio (DAR) of the antibody-linker-conjugate produced in step (2) is between 3 and 4.5, preferably 3.8 to 4.1.

34. The method according to claim 1, wherein the drug antibody ratio (DAR) of the antibody-linker-conjugate produced in step (2) is between about 1.9 and 2.

35. The method of claim 34, wherein said antibody comprises the amino acid Q295 in its heavy chain constant regions (CH), wherein Eu numbering is used for defining the position of said glutamine at position 295; and wherein said antibody is an IgG monoclonal antibody.

36. The method of claim 34, wherein the microbial transglutaminase is from S. ladakanum; wherein said antibody is a monoclonal antibody comprising glutamine at position 295 and wherein the reaction buffer comprises HEPES in an amount of between about 20 to 30 mM; wherein the reaction buffer comprises no NaCl and wherein the pH of the reaction buffer is between about pH 7 and pH 7.5; and wherein Eu numbering is used for defining the position of said glutamine at position 295.

37. The method of claim 34, wherein the microbial transglutaminase is from S. ladakanum; wherein said antibody is a monoclonal antibody comprising glutamine at position 295 and wherein the reaction buffer comprises HEPES in an amount of between about 20 to 30 mM; wherein the reaction buffer comprises no NaCl and wherein the pH of the reaction buffer is between about pH 7 and pH 7.5; and wherein Eu numbering is used for defining the position of said glutamine at position 295; and wherein the C-terminus of the light chain of the antibody comprises the amino acid sequence GGTLQSPP.

38. The method according to claim 1, wherein the linker has a structure (A) or (B) shown below, wherein R is a therapeutic agent or another payload:

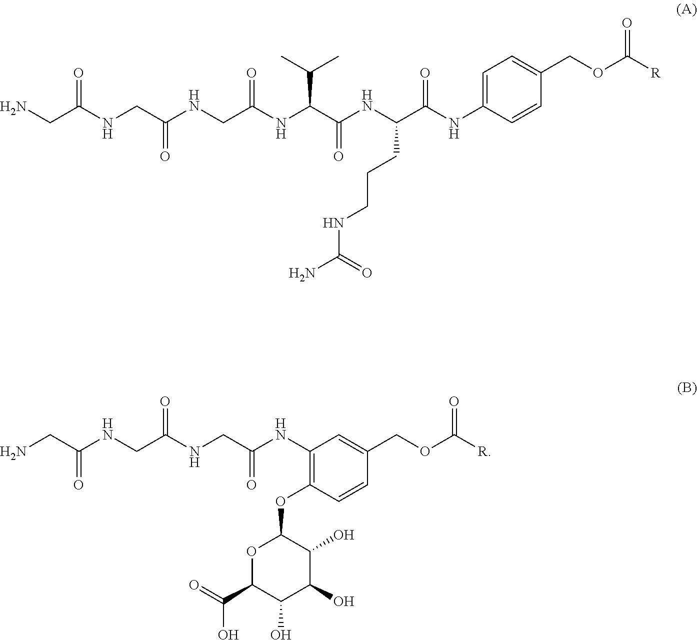

39. The method according to claim 1, wherein the antibody comprises one or more amino acid sequence selected from the group consisting of GGTLQSPP, TLQSG, TLQSPP, GGTLQSG and TLQSA; wherein this amino acid sequence is located at the C-terminus of the light chain of the antibody and/or at the C-terminus of the heavy chain of the antibody.

40. The method according to claim 1, wherein in step (2) a molar excess of drug linker is used per conjugation site of the antibody and preferably at least 4 molar equivalents of drug-linker per conjugation site.

41. (canceled)

42. (canceled)

43. (canceled)

44. (canceled)

45. An antibody comprising an amino acid sequence selected from the group consisting of GGTLQSPP, GGTLQSG and TLQSA at the C-terminus of the heavy chain of the antibody and/or comprises an amino acid sequence selected from the group consisting of GGTLQSPP, GGTLQSG and TLQSA at the C-terminus of the light chain of the antibody.


SEQUENCE LISTING (84 sequences)

SEQ ID NO: 1 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 2 - protein, 705 aa, Macaca fascicularis
MGSPSAPLHRWCIPWQTLLLTASLLTFWNPPTTAQLTIESRPFNVAEGKEVLLLAHNVSQNLFGYIWYKGERVDASRRIGSCVIRTQQITPGPAHSGRETIDFNASLLIHNVTQSDTGSYTIQVIKEDLVNEEATGQFRVYPELPKPYISSNNSNPVEDKDAVALTCEPETQDTTYLWWVNNQSLPVSPRLELSSDNRTLTVFNIPRNDTTSYKCETQNPVSVRRSDPVTLNVLYGPDAPTISPLNTPYRAGENLNLSCHAASNPTAQYFWFVNGTFQQSTQELFIPNITVNNSGSYMCQAHNSATGLNRTTVTAITVYAELPKPYITSNNSNPIEDKDAVTLTCEPETQDTTYLWWVNNQSLSVSSRLELSNDNRTLTVFNIPRNDTTFYECETQNPVSVRRSDPVTLNVLYGPDAPTISPLNTPYRAGENLNLSCHAASNPAAQYSWFVNGTFQQSTQELFIPNITVNNSGSYMCQAHNSATGLNRTTVTAITVYVELPKPYISSNNSNPIEDKDAVTLTCEPVAENTTYLWWVNNQSLSVSPRLQLSNGNRILTLLSVTRNDTGPYECGIQNSESAKRSDPVTLNVTYGPDTPIISPPDLSYRSGANLNLSCHSDSNPSPQYSWLINGTLRQHTQVLFISKITSNNNGAYACFVSNLATGRNNSIVKNISVSSGDSAPGSSGLSARATVGIIIGMLVGVALM

SEQ ID NO: 3 - protein, 10 aa, synthetic construct
DGSVSRGGYY

SEQ ID NO: 4 - protein, 7 aa, synthetic construct
IYYSGST

SEQ ID NO: 5 - protein, 11 aa, synthetic construct
ARGIAVAPFDY

SEQ ID NO: 6 - protein, 6 aa, synthetic construct
QSVRSN

SEQ ID NO: 7 - skipped

SEQ ID NO: 8 - protein, 9 aa, synthetic construct
QQYTNWPFT

SEQ ID NO: 9 - protein, 119 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSS

SEQ ID NO: 10 - protein, 107 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIK

SEQ ID NO: 11 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 107 aa, synthetic construct [sequence omitted]

SEQ ID NO: 13 - protein, 447 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 214 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 1404 bp, synthetic construct
  source 1..1404: mol_type = other DNA
atggagaccgacaccctgctgctgtgggtgctgctgctgtgggtgcccgggtcgaccggtgaggtgcagctgcaggagtcgggcccaggactggtgaagccttcacagaccctgtccctcacctgcactgtctctgatggctccgtcagcaggggtggttactacttgacctggatccgccagcacccagggaagggcctggagtggattgggtacatctattacagtgggagcacctacttcaacccgtccctcaggagtcgggttaccatgtcagtagacacgtctaagaaccagttctccctgaagctgagctctgtgactgccgcggacacggccgtgtattactgtgcgagagggatagcagtggctccctttgactactggggccagggaaccctggtcaccgtctcttcagctagcaccaagggccccagcgtgttccccctggcccccagcagcaagtccacaagcggaggaacagccgccctgggctgcctggtgaaggactacttccccgagcccgtgaccgtgtcctggaacagcggagccctgacctccggcgtgcacaccttccccgccgtgctgcagagcagcggcctgtacagcctgagcagcgtggtgacagtgccaagcagcagcctgggaacccagacctacatctgcaacgtgaaccacaagcccagcaacaccaaggtggacaagagagtggagcccaagagctgcgacaagacccatacctgtccaccctgcccagcccccccagtggccggaccctccgtgttcctgttcccccccaagcccaaggacaccctgatgatcagcaggacccccgaggtgacctgcgtggtggtggacgtgagccacgaggacccagaggtgaagttcaattggtatgtggacggcgtggaggtgcacaacgccaagaccaagcccagagaggaacagtacaacagcacctacagggtggtgtccgtgctgaccgtgctgcaccaggactggctgaacggcaaggaatacaagtgcaaggtctccaacaaggccctgccctccagcatcgagaaaaccatcagcaaggccaagggccagccacgggagccccaggtgtacacactgcccccatctcgggaagaaatgaccaagaaccaggtgtccctgacctgtctggtgaagggcttttaccccagcgacatcgccgtggagtgggagagcaacggccagcccgagaacaactacaagaccaccccccctgtgctggacagcgacggcagcttcttcctgtacagcaagctgaccgtggacaagtccaggtggcagcagggcaacgtgttcagctgcagcgtgatgcacgaggccctgcacaaccactacacacagaagagcctgagcctgtcccccggctga

SEQ ID NO: 16 - DNA, 708 bp, synthetic construct
  source 1..708: mol_type = other DNA
atgagggccctgctggctagactgctgctgtgcgtgctggtcgtgtccgacagcaagggcgaaatcgtactcacgcagtctccagccaccctgtctgtgtctccaggggaaagagccaccctctcctgcaggaccagtcagagtgttcgcagcaacttagcctggtaccagcagaagcctggccaggctcccaggctcctcatctatgctgcatccaccagggccactggtatcccagccaggttcagtggcagtgggtctgggacagagttcactctcaccatcagcagcctgcagtctgaagattttgcagtttattactgtcagcagtatactaactggccattcactttcggccctgggaccaaagtggacatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatag

SEQ ID NO: 17 - protein, 449 aa, synthetic construct
EVQLVESGPGLVKPSQTLSLTCTVSDGSVSRGGYYLTWIRQHPGKGLEWIGYIYYSGSTYFNPSLRSRLTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 214 aa, synthetic construct
ETTLTQSPATLSVSPGERATLSCRTSQSVRSNLAWYQQKPGQAPRLLIYAASTRATGIPARFSGSGSGTEFTLTIGSLQSEDFAVYFCQQYTNWPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, synthetic construct
EVQLQESGPGLVKPSQTLSLTCTVSDGSVSRGGYYLTWIRQHPGKGLEWIGYIYYSGSTYFNPSLRSRLTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct; identical to SEQ ID NO: 18

SEQ ID NO: 21 - protein, 214 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYFCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, synthetic construct
EIVMTQSPATLSVSPGERATLSCRTSQSVRSNLAWYQQKPGQAPRLLIYAASTRATGIPARFSGSGSGTEFTLTISSLQSEDFAVYFCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, synthetic construct
EIVMTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 449 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 450 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 449 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 441 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 30 - protein, 213 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIKRDPVAPSVLLFPPSKEELTTGTATIVCVANKFYPSDITVTWKVDGTTQQSGIENSKTPQSPEDNTYSLSSTLSLTSAQYNSHSVYTCEVVQGSASPIVQSFNRGDC

SEQ ID NO: 31 - protein, 329 aa, synthetic construct
  REGION 1..329: note = CH of mAb1-M and of mAb2-M (CH-LALA-YTE)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDRTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 115 aa, synthetic construct
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGTLQSPP

SEQ ID NO: 34 - protein, 448 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 448 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDRTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 222 aa, synthetic construct
EIVLTQSPATLSVSPGERATLSCRTSQSVRSNLAWYQQKPGQAPRLLIYAASTRATGIPARFSGSGSGTEFTLTISSLQSEDFAVYYCQQYTNWPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGTLQSPP

SEQ ID NO: 37 - DNA, 1407 bp, synthetic construct
  source 1..1407: mol_type = other DNA
atgaagctgcctgtcagactgctggtgctgatgttctggattcccgcctctctgtccgaggtccagctgcaagaatctggccctggactggtcaagccctctcagaccctgtctctgacctgcaccgtgtctgacggctctgtttctagaggcggctactacctgacctggatcagacagcatcctggcaagggcctcgagtggatcggctacatctactactccggctccacctacttcaaccccagcctgagatccagagtgaccatgtccgtggacacctccaagaaccagttctccctgaagctgtcctccgtgaccgctgctgataccgccgtgtactactgcgccagaggaatcgccgtggctccctttgattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatcctccggcctgtactctctgagcagcgtcgtgaccgtgccttctagctctctgggcacccagacctacatctgcaatgtgaaccacaagccttccaacaccaaggtggacaagagagtggaacccaagtcctgcgacaagacccacacctgtcctccatgtcctgctccagaagctgctggcggcccttccgtgtttctgttccctccaaagcctaaggacaccctgtacatcacccgcgagcctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaatcaggtgtccctgacctgtctcgtgaagggcttctacccctccgatatcgctgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagagtctgtccctgtctcctggctgatga

SEQ ID NO: 38 - DNA, 1407 bp, synthetic construct
  source 1..1407: mol_type = other DNA
atgaagctgcctgtcagactgctggtgctgatgttctggattcccgcctctctgtccgaggtccagctgcaagaatctggccctggactggtcaagccctctcagaccctgtctctgacctgcaccgtgtctgacggctctgtttctagaggcggctactacctgacctggatcagacagcatcctggcaagggcctcgagtggatcggctacatctactactccggctccacctacttcaaccccagcctgagatccagagtgaccatgtccgtggacacctccaagaaccagttctccctgaagctgtcctccgtgaccgctgctgataccgccgtgtactactgcgccagaggaatcgccgtggctccctttgattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatcctccggcctgtactctctgagcagcgtcgtgaccgtgccttctagctctctgggcacccagacctacatctgcaatgtgaaccacaagccttccaacaccaaggtggacaagagagtggaacccaagtcctgcgacagaacccacacctgtcctccatgtcctgctccagaagctgctggcggcccttccgtgtttctgttccctccaaagcctaaggacaccctgtacatcacccgcgagcctgaagtgacctgcgtggtggtggatgtgtctcacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaatcaggtgtccctgacctgtctcgtgaagggcttctacccctccgatatcgctgtggaatgggagagcaatggccagccagagaacaactacaagaccacacctcctgtgctggactccgacggctcattcttcctgtactccaagctgacagtggacaagtccagatggcagcagggcaacgtgttctcctgctccgtgatgcacgaggccctgcacaatcactacacccagaagagtctgtccctgtctcctggctgatga

SEQ ID NO: 39 - DNA, 729 bp, synthetic construct
  source 1..729: mol_type = other DNA
atgaagctgcctgtcagactgctggtgctgatgttctggattcccgccagcctgtccgagatcgtgctgactcaatctcccgccacactgtctgtgtctcccggcgaaagagctaccctgtcttgccggacatctcagtccgtgcggtctaacctggcctggtatcagcagaagcctggacaggctcctcggctgctgatctacgctgcttctaccagagccacaggcatccccgccagattttctggctctggatctggcaccgagtttaccctgaccatctccagcctgcagtccgaggatttcgccgtgtactactgccagcagtataccaactggcccttcaccttcggacccggcacaaaggtggacatcaagagaaccgtggccgctccttccgtgttcatcttcccaccttccgacgagcagctgaagtccggcacagcttctgtcgtgtgcctgctgaacaacttctaccctcgggaagccaaggtgcagtggaaagtggataacgccctgcagagcggcaactcccaagagtctgtgaccgagcaggactccaaggacagcacctacagcctgagcagcaccctgacactgtccaaggccgactacgagaagcacaaggtgtacgcctgcgaagtgacccatcagggcctgtctagccctgtgaccaagtctttcaacagaggcgagtgcggcggcaccttgcagtctccaccttgataa

SEQ ID NO: 40 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 221 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGTLQSPP

SEQ ID NO: 42 - DNA, 1416 bp, synthetic construct
  source 1..1416: mol_type = other DNA
atgaagctgcctgtcagactgctggtgctgatgttctggattcccgccagcctgtctcaggtccagttgcaacaacctggcgccgagcttgtgaagcctggcgcttctgtgaagatgtcctgcaaggcctccggctacaccttcaccagctacaacatgcactgggtcaagcagacccctggcagaggcctggaatggatcggcgctatctatcctggcaacggcgacacctcctacaaccagaagttcaagggcaaagctaccctgaccgccgacaagtcctcttccaccgcttacatgcagctgtccagcctgacctctgaggactccgccgtgtactactgcgcccggtctacctattatggcggcgactggtacttcaacgtgtggggcgctggcaccaccgtgacagtttctgccgcttctaccaagggacccagcgtgttccctctggctccttccagcaagtctacctctggcggaacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagagagtggaacccaagtcctgcgacaagacccacacctgtcctccatgtcctgctccagaagctgctggcggcccttccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggcaagtgatga

SEQ ID NO: 43 - DNA, 726 bp, synthetic construct
  source 1..726: mol_type = other DNA
atgaagctgcctgtcagactgctggtgctgatgttctggattcccgcctctctgtcccagatcgtgctgtctcagtctcccgccatcctgagtgctagccctggcgagaaagtgacaatgacctgcagagcctcctcctccgtgtcctacatccactggttccagcagaagcctggcagctcccctaagccttggatctacgccacctccaatctggcctctggcgtgccagtcagattctccggatctggctccggcacctcctacagcctgaccatctctagagtggaagccgaggatgccgccacctactactgtcagcagtggaccagcaatcctcctacctttggcggaggcaccaagctggaaatcaagcggacagtggccgctccttccgtgttcatcttcccaccttccgacgagcagctgaagtctggcacagcctctgtcgtgtgcctgctgaacaacttctaccctcgggaagccaaggtgcagtggaaggtggacaacgctctgcagtccggcaactcccaagagtctgtgaccgagcaggactccaaggactctacctactctctgagcagcaccctgacactgtccaaggccgactacgagaagcacaaggtgtacgcctgcgaagtgacccatcagggcctgtctagccctgtgaccaagtctttcaacagaggcgagtgtggcggcaccctgcagtctccaccttgataa

SEQ ID NO: 44 - protein, 381 aa, synthetic construct
MGSGSGSGTGEEKRSYAETHRLTADDVDDINALNESAPAASSAGPSFRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 45 - protein, 335 aa, synthetic construct
FRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 46 - DNA, 1146 bp, synthetic construct
  source 1..1146: mol_type = other DNA
atgggtagcggtagcggttcaggcaccggtgaagaaaaacgtagctatgcagaaacccatcgtctgaccgcagatgatgttgatgatattaatgcactgaatgaaagcgcaccggcagcaagcagcgcaggtccgagctttcgtgcaccggatagtgatgaacgtgttacccctccggcagaaccgctggatcgtatgccggatccgtatcgtccgagctatggtcgtgccgaaaccattgttaataactatattcgtaagtggcagcaggtttatagccatcgtgatggtcgtaaacagcagatgaccgaagaacagcgtgaatggctgagttatggttgtgttggtgttacctgggttaatagcggtcagtatccgaccaatcgtctggcatttgcatttttcgatgaggacaaatacaagaacgagctgaaaaatggtcgtccgcgtagcggtgaaacccgtgcagaatttgaaggtcgtgttgcaaaagatagctttgatgaagcaaaaggttttcagcgtgcacgtgatgttgcaagcgttatgaataaagcactggaaaatgcacatgatgaaggtgcctatctggacaacctgaaaaaagaactggcaaatggtaatgatgccctgcgtaatgaagatgcacgtagcccgttttatagcgcactgcgtaataccccgagctttaaagatcgtaatggtggtaatcatgacccgagcaaaatgaaagcagtgatctatagcaaacacttttggagcggtcaggatcgtagtggtagcagcgataaacgtaaatatggtgatccggaagcatttcgtccggatcgtggcaccggtctggttgatatgagccgtgatcgtaatattccgcgtagtccgaccagtccgggtgaaagctttgttaattttgattatggttggtttggcgcacagaccgaagcagatgcagataaaaccgtttggacccatggcaatcattatcatgcaccgaatggtagcctgggtgcaatgcatgtttatgaaagcaaatttcgcaattggagcgacggctatagcgattttgatcgtggtgcctatgttgttacctttgttccgaaaagctggaataccgctccggataaagttacccaaggttggccgtaa

SEQ ID NO: 47 - protein, 337 aa, synthetic construct
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPLE

SEQ ID NO: 48 - protein, 333 aa, synthetic construct
DSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWPLE

SEQ ID NO: 49 - protein, 331 aa, synthetic construct
DSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWP

SEQ ID NO: 50 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 448 aa, synthetic construct
EVQLQESGPGLVKPSQTLSLTCTVSDGSVSRGGYYLTWIRQHPGKGLEWIGYIYYSGSTYFNPSLRSRVTMSVDTSKNQFSLKLSSVTAADTAVYYCARGIAVAPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - DNA, 1407 bp, synthetic construct
  source 1..1407: mol_type = other DNA
atgaagctgcctgtcagactgctggtgctgatgttctggattcccgcctctctgtccgaggtccagctgcaagaatctggccctggactggtcaagccctctcagaccctgtctctgacctgcaccgtgtctgacggctctgtttctagaggcggctactacctgacctggatcagacagcatcctggcaagggcctcgagtggatcggctacatctactactccggctccacctacttcaaccccagcctgagatccagagtgaccatgtccgtggacacctccaagaaccagttctccctgaagctgtcctccgtgaccgctgctgataccgccgtgtactactgcgccagaggaatcgccgtggctccctttgattattggggccagggcaccctggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatccggcgtgcacacctttccagctgtgctgcaatcctccggcctgtactctctgagcagcgtcgtgaccgtgccttctagctctctgggcacccagacctacatctgcaatgtgaaccacaagccttccaacaccaaggtggacaagagagtggaacccaagtcctgcgacaagacccacacctgtcctccatgtcctgctccagaagctgctggcggcccttccgtgtttctgttccctccaaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccctgcctccaagccgggaagagatgaccaagaatcaggtgtccctgacctgtctcgtgaagggcttctacccctccgatatcgctgtggaatgggagagcaatggccagccagagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagagtctgtccctgtctcctggctgatga

SEQ ID NO: 53 - DNA, 708 bp, synthetic construct
  source 1..708: mol_type = other DNA
atggagaccgacaccctgctgctgtgggtgctgctgctgtgggtgcccgggtcgaccggtgaaatcgtactcacgcagtctccagccaccctgtctgtgtctccaggggaaagagccaccctctcctgcaggaccagtcagagtgttcgcagcaacttagcctggtaccagcagaagcctggccaggctcccaggctcctcatctatgctgcatccaccagggccactggtatcccagccaggttcagtggcagtgggtctgggacagagttcactctcaccatcagcagcctgcagtctgaagattttgcagtttattactgtcagcagtatactaactggccattcactttcggccctgggaccaaagtggacatcaaacgtacggtggcagcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttgatag

SEQ ID NO: 54 - protein, 25 aa, synthetic construct
EVQLQESGPGLVKPSQTLSLTCTVS

SEQ ID NO: 55 - protein, 17 aa, synthetic construct
LTWIRQHPGKGLEWIGY

SEQ ID NO: 56 - protein, 38 aa, synthetic construct
YFNPSLRSRVTMSVDTSKNQFSLKLSSVTAADTAVYYC

SEQ ID NO: 57 - protein, 11 aa, synthetic construct
WGQGTLVTVSS

SEQ ID NO: 58 - protein, 26 aa, synthetic construct
EIVLTQSPATLSVSPGERATLSCRTS

SEQ ID NO: 59 - protein, 17 aa, synthetic construct
LAWYQQKPGQAPRLLIY

SEQ ID NO: 60 - protein, 36 aa, synthetic construct
TRATGIPARFSGSGSGTEFTLTISSLQSEDFAVYYC

SEQ ID NO: 61 - protein, 10 aa, synthetic construct
FGPGTKVDIK

SEQ ID NO: 62 - protein, 662 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 213 aa, synthetic construct; identical to SEQ ID NO: 28

SEQ ID NO: 64 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 455 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEAYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGTLQSG

SEQ ID NO: 68 - protein, 221 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGTLQSG

SEQ ID NO: 69 - protein, 222 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGTLQSPP

SEQ ID NO: 70 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDRTHTCPPCPAPELLGGPSVFLFPPKPKDTLMISRTPEVTCVVVDVSHEDPEVKFNWYVDGVEVHNAKTKPREEAYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 221 aa, synthetic construct; identical to SEQ ID NO: 41

SEQ ID NO: 72 - protein, 448 aa, synthetic construct; identical to SEQ ID NO: 51

SEQ ID NO: 73 - protein, 337 aa, synthetic construct
FRAPDSDERVTPPAEPLDRMPDPYRPSYGRAETIVNNYIRKWQQVYSHRDGRKQQMTEEQREWLSYGCVGVTWVNSGQYPTNRLAFAFFDEDKYKNELKNGRPRSGETRAEFEGRVAKDSFDEAKGFQRARDVASVMNKALENAHDEGAYLDNLKKELANGNDALRNEDARSPFYSALRNTPSFKDRNGGNHDPSKMKAVIYSKHFWSGQDRSGSSDKRKYGDPEAFRPDRGTGLVDMSRDRNIPRSPTSPGESFVNFDYGWFGAQTEADADKTVWTHGNHYHAPNGSLGAMHVYESKFRNWSDGYSDFDRGAYVVTFVPKSWNTAPDKVTQGWPLE

SEQ ID NO: 74 - protein, 8 aa, synthetic construct
GGTLQSPP

SEQ ID NO: 75 - protein, 5 aa, synthetic construct
LLQGA

SEQ ID NO: 76 - protein, 8 aa, synthetic construct
GGLLQGPP

SEQ ID NO: 77 - protein, 5 aa, synthetic construct
TLQSG

SEQ ID NO: 78 - protein, 6 aa, synthetic construct
TLQSPP

SEQ ID NO: 79 - protein, 7 aa, synthetic construct
GGTLQSG

SEQ ID NO: 80 - protein, 5 aa, synthetic construct
TLQSA

SEQ ID NO: 81 - protein, 331 aa, synthetic construct
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 82 - protein, 333 aa, synthetic construct
DSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWPLE

SEQ ID NO: 83 - protein, 335 aa, synthetic construct
FRAPDSDDRVTPPAEPLDRMPDPYRPSYGRAETVVNNYIRKWQQVYSHRDGRKQQMTEEQREWLSYGCVGVTWVNSGQYPTNRLAFASFDEDRFKNELKNGRPRSGETRAEFEGRVAKESFDEEKGFQRAREVASVMNRALENAHDESAYLDNLKKELANGNDALRNEDARSPFYSALRNTPSFKERNGGNHDPSRMKAVIYSKHFWSGQDRSSSADKRKYGDPDAFRPAPGTGLVDMSRDRNIPRSPTSPGEGFVNFDYGWFGAQTEADADKTVWTHGNHYHAPNGSLGAMHVYESKFRNWSEGYSDFDRGAYVITFIPKSWNTAPDKVKQGWP

SEQ ID NO: 84 - protein, 337 aa, synthetic construct; identical to SEQ ID NO: 47